(19) United States
(12) Patent Application Publication
(10) Pub. No.: US 2024/0209359 A1
(43) Pub. Date: Jun. 27, 2024

(54) METHODS, SYSTEMS, AND APPARATUS FOR IDENTIFYING TARGET SEQUENCES FOR CAS ENZYMES OR CRISPR-CAS SYSTEMS FOR TARGET SEQUENCES AND CONVEYING RESULTS THEREOF

(71) Applicants: The Broad Institute, Inc., Cambridge, MA (US); Massachusetts Institute of Technology, Cambridge, MA (US)

(72) Inventors: Feng ZHANG, Cambridge, MA (US); Naomi HABIB, Cambridge, MA (US)

(73) Assignees: The Broad Institute, Inc., Cambridge, MA (US); Massachusetts Institute of Technology, Cambridge, MA (US)

(21) Appl. No.: 18/225,531

(22) Filed: Jul. 24, 2023

Related U.S. Application Data
(63) Continuation of application No. 16/012,692, filed on Jun. 19, 2018, now abandoned.
Application No. 16/012,692 is a continuation of application No. 14/104,900, filed on Dec. 12, 2013, now abandoned.
(60) Provisional application No. 61/736,527, filed on Dec. 12, 2012.
(60) Provisional application No. 61/748,427, filed on Jan. 2, 2013.
(60) Provisional application No. 61/791,409, filed on Mar. 15, 2013.
(60) Provisional application No. 61/835,931, filed on Jun. 17, 2013.

(51) Int. Cl. C12N 15/113; C12N 9/22; C12N 15/10; C12N 15/63; C12N 15/79; C12N 15/90; G16B 20/00; G16B 20/20; G16B 20/30; G16B 20/50; G16B 30/00; G16B 30/10

(52) U.S. Cl. CPC C12N 15/113; C12N 9/22; C12N 15/102; C12N 15/1082; C12N 15/63; C12N 15/907; G16B 20/00; G16B 20/20; G16B 20/30; G16B 20/50; G16B 30/10; C12N 15/79; C12N 2310/10; C12N 2310/20; C12N 2320/11; C12N 2320/30; C12N 2750/14143; G16B 30/00

(57) ABSTRACT

Disclosed are locational or positional methods concerning CRISPR-Cas systems, and apparatus therefor.

### RELATED APPLICATIONS AND INCORPORATION BY REFERENCE

This application is a continuation of U.S. application Ser. No. 16/012,692, filed Jun. 19, 2018, which is a continuation of U.S. application Ser. No. 14/104,900 entitled METHODS, SYSTEMS, AND APPARATUS FOR IDENTIFYING TARGET SEQUENCES FOR CAS ENZYMES OR CRISPR-CAS SYSTEMS FOR TARGET SEQUENCES AND CONVEYING RESULTS THEREOF filed on Dec. 12, 2013; claims priority to U.S. provisional patent applications 61/736,527, 61/748,427 and 61/791,409 all entitled SYSTEMS METHODS AND COMPOSITIONS FOR SEQUENCE MANIPULATION filed on Dec. 12, 2012, Jan. 2, 2013 and Mar. 15, 2013, respectively. Priority is also claimed to U.S. provisional patent application 61/835,931 entitled SYSTEMS METHODS AND COMPOSITIONS FOR SEQUENCE MANIPULATION filed on Jun. 17, 2013.

Reference is made to U.S. provisional patent applications 61/758,468; 61/769,046; 61/802,174; 61/806,375; 61/814,263; 61/819,803 and 61/828,130, each entitled ENGINEERING AND OPTIMIZATION OF SYSTEMS, METHODS AND COMPOSITIONS FOR SEQUENCE MANIPULATION, filed on Jan. 30, 2013; Feb. 25, 2013; Mar. 15, 2013; Mar. 28, 2013; Apr. 20, 2013; May 6, 2013 and May 28, 2013 respectively. Reference is also made to U.S. provisional patent application 61/791,409 entitled SYSTEMS METHODS AND COMPOSITIONS FOR SEQUENCE MANIPULATION filed on Mar. 15, 2013. Reference is also made to U.S. provisional patent applications 61/836,127, 61/835,936, 61/836,080, 61/836,101 and 61/835,973 each filed Jun. 17, 2013.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### STATEMENT AS TO FEDERALLY SPONSORED RESEARCH

This invention was made with government support under Grant Nos. MH100706 and DK097768 awarded by the National Institutes of Health. The government has certain rights in the invention.

### FIELD OF THE INVENTION

The present invention generally relates to the engineering and optimization of systems, methods and compositions used for the control of gene expression involving sequence targeting, such as genome perturbation or gene-editing, that relate to Clustered Regularly Interspaced Short Palindromic Repeats (CRISPR) and components thereof.

### SEQUENCE LISTING

The instant application contains a Sequence Listing which has been submitted electronically in ASCII format and is hereby incorporated by reference in its entirety. Said ASCII copy, created on Feb. 27, 2014, is named 44790.00.2040_SL.txt and is 239,402 bytes in size.

### BACKGROUND OF THE INVENTION

The CRISPR/Cas or the CRISPR-Cas system (both terms are used interchangeably throughout this application) does not require the generation of customized proteins to target specific sequences but rather a single Cas enzyme can be programmed by a short RNA molecule to recognize a specific DNA target. Adding the CRISPR-Cas system to the repertoire of genome sequencing techniques and analysis methods may significantly simplify the methodology and accelerate the ability to catalog and map genetic factors associated with a diverse range of biological functions and diseases. To utilize the CRISPR-Cas system effectively for genome editing without deleterious effects, it is critical to understand methods, systems and apparatus for identifying target sequences for Cas enzymes or CRISPR-Cas systems for target sequences of interest and conveying the results, which are aspects of the claimed invention.

### SUMMARY OF THE INVENTION

The CRISPR/Cas or the CRISPR-Cas system (both terms may be used interchangeably throughout this application) does not require the generation of customized proteins to target specific sequences but rather a single Cas enzyme can be programmed by a short RNA molecule to recognize a specific DNA target, in other words the Cas enzyme can be recruited to a specific DNA target using said short RNA molecule. Adding the CRISPR-Cas system to the repertoire of genome sequencing techniques and analysis methods may significantly simplify the methodology and accelerate the ability to catalog and map genetic factors associated with a diverse range of biological functions and diseases. To utilize the CRISPR-Cas system effectively for genome editing without deleterious effects, it is critical to understand aspects of engineering and optimization of these genome engineering tools, which are aspects of the claimed invention.

In some aspects the invention relates to a non-naturally occurring or engineered composition comprising a CRISPR/Cas system chimeric RNA (chiRNA) polynucleotide sequence, wherein the polynucleotide sequence comprises (a) a guide sequence capable of hybridizing to a target sequence in a eukaryotic cell, (b) a tracr mate sequence, and (c) a tracr sequence wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence, wherein the CRISPR complex comprises a CRISPR enzyme complexed with (1) the guide sequence that is hybridized to the target sequence, and (2) the tracr mate sequence that is hybridized to the tracr sequence, or

an CRISPR enzyme system, wherein the system is encoded by a vector system comprising one or more vectors comprising I. a first regulatory element operably linked to a CRISPR/Cas system chimeric RNA (chiRNA) polynucleotide sequence, wherein the polynucleotide sequence comprises (a) one or more guide sequences capable of hybridizing to one or more target sequences in a eukaryotic cell, (b) a tracr mate sequence, and (c) one or more tracr sequences, and II. a second regulatory element operably linked to an enzyme-coding sequence encoding a CRISPR enzyme comprising at least one or more nuclear localization sequences, wherein (a), (b) and (c) are arranged in a 5′ to 3′orientation, wherein components I and II are located on the same or different vectors of the system, wherein when transcribed, the tracr mate sequence hybridizes to the tracr sequence and the guide sequence directs sequence-specific binding of a CRISPR complex to the target sequence, wherein the CRISPR complex comprises the CRISPR enzyme complexed with (1) the guide sequence that is hybridized to the target sequence, and (2) the tracr mate sequence that is hybridized to the tracr sequence, or

a multiplexed CRISPR enzyme system, wherein the system is encoded by a vector system comprising one or more vectors comprising I. a first regulatory element operably linked to (a) one or more guide sequences capable of hybridizing to a target sequence in a cell, and (b) at least one or more tracr mate sequences, II. a second regulatory element operably linked to an enzyme-coding sequence encoding a CRISPR enzyme, and III. a third regulatory element operably linked to a tracr sequence, wherein components I, II and III are located on the same or different vectors of the system, wherein when transcribed, the tracr mate sequence hybridizes to the tracr sequence and the guide sequence directs sequence-specific binding of a CRISPR complex to the target sequence, wherein the CRISPR complex comprises the CRISPR enzyme complexed with (1) the guide sequence that is hybridized to the target sequence, and (2) the tracr mate sequence that is hybridized to the tracr sequence, and wherein in the multiplexed system multiple guide sequences and a single tracr sequence is used.

Without wishing to be bound by theory, it is believed that the target sequence should be associated with a PAM (protospacer adjacent motif); that is, a short sequence recognized by the CRISPR complex. This PAM may be considered a CRISPR motif.

With regard to the CRISPR system or complex discussed herein, reference is made to FIG. 2. FIG. 2 shows an exemplary CRISPR system and a possible mechanism of action (A), an example adaptation for expression in eukaryotic cells, and results of tests assessing nuclear localization and CRISPR activity (B-F).

The invention provides a method of identifying one or more unique target sequences. The target sequences may be in a genome of an organism, such as a genome of a eukaryotic organism. Accordingly, through potential sequence-specific binding, the target sequence may be susceptible to being recognized by a CRISPR-Cas system. (Likewise, the invention thus comprehends identifying one or more CRISPR-Cas systems that identifies one or more unique target sequences.) The target sequence may include the CRISPR motif and the sequence upstream or before it. The method may comprise: locating a CRISPR motif, e.g., analyzing (for instance comparing) a sequence to ascertain whether a CRISPR motif, e.g., a PAM sequence, a short sequence recognized by the CRISPR complex, is present in the sequence; analyzing (for instance comparing) the sequence upstream of the CRISPR motif to determine if that upstream sequence occurs elsewhere in the genome; selecting the upstream sequence if it does not occur elsewhere in the genome, thereby identifying a unique target site. The sequence upstream of the CRISPR motif may be at least 10 bp or at least 11 bp or at least 12 bp or at least 13 bp or at least 14 bp or at least 15 bp or at least 16 bp or at least 17 bp or at least 18 bp or at least 19 bp or at least 20 bp in length, e.g., the sequence upstream of the CRISPR motif may be about 10 bp to about 20 bp, e.g., the sequence upstream is 10, 11, 12, 13, 14, 15, 16, 17, 18, 19, 20, 21, 22, 23, 24, 25, 26, 27, 28, 29 or 30 bp in length. The CRISPR motif may be recognized by a Cas enzyme such as a Cas9 enzyme, e.g., a SpCas9 enzyme. Further, the CRISPR motif may be a protospacer-adjacent motif (PAM) sequence, e.g., NGG or NAG. Accordingly, as CRISPR motifs or PAM sequences may be recognized by a Cas enzyme in vitro, ex vivo or in vivo, in the in silico analysis, there is an analysis, e.g., comparison, of the sequence in interest against CRISPR motifs or PAM sequences to identify regions of the sequence in interest which may be recognized by a Cas enzyme in vitro, ex vivo or in vivo. When that analysis identifies a CRISPR motif or PAM sequence, the next analysis e.g., comparison is of the sequences upstream from the CRISPR motif or PAM sequence, e.g., analysis of the sequence 10, 11, 12, 13, 14, 15, 16, 17, 18, 19, 20, 21, 22, 23, 24, 25, 26, 27, 28, 29 or 30 bp in length starting at the PAM or CRISPR motif and extending upstream therefrom. That analysis is to see if that upstream sequence is unique, i.e., if the upstream sequence does not appear to otherwise occur in a genome, it may be a unique target site. The selection for unique sites is the same as the filtering step: in both cases, you filter away all target sequences with associated CRISPR motif that occur more than once in the target genome.

Eukaryotic organisms of interest may include but are not limited to Homo sapiens (human), Mus musculus (mouse), Rattus norvegicus (rat), Danio rerio (zebrafish), Drosophila melanogaster (fruit fly), Caenorhabditis elegans (roundworm), Sus scrofa (pig) and Bos taurus (cow). The eukaryotic organism can be selected from the group consisting of Homo sapiens (human), Mus musculus (mouse), Rattus norvegicus (rat), Danio rerio (zebrafish), Drosophila melanogaster (fruit fly), Caenorhabditis elegans (roundworm), Sus scrofa (pig) and Bos taurus (cow). The invention also comprehends computer-readable medium comprising codes that, upon execution by one or more processors, implements a herein method of identifying one or more unique target sequences.

The invention further comprehends a computer system for identifying one or more unique target sequences, e.g., in a genome, such as a genome of a eukaryotic organism, the system comprising: a. a memory unit configured to receive and/or store sequence information of the genome; and b. one or more processors alone or in combination programmed to perform a herein method of identifying one or more unique target sequences (e.g., locate a CRISPR motif, analyze a sequence upstream of the CRISPR motif to determine if the sequence occurs elsewhere in the genome, select the sequence if it does not occur elsewhere in the genome), to thereby identifying a unique target site and display and/or transmit the one or more unique target sequences. The candidate target sequence may be a DNA sequence. Mismatch(es) can be of RNA of the CRISPR complex and the DNA. In aspects of the invention, susceptibility of a target sequence being recognized by a CRISPR-Cas system indicates that there may be stable binding between the one or more base pairs of the target sequence and guide sequence of the CRISPR-Cas system to allow for specific recognition of the target sequence by the guide sequence.

The CRISPR/Cas or the CRISPR-Cas system utilizes a single Cas enzyme that can be programmed by a short RNA molecule to recognize a specific DNA target, in other words the Cas enzyme can be recruited to a specific DNA target using said short RNA molecule. In certain aspects, e.g., when not mutated or modified or when in a native state, the Cas or CRISPR enzyme in CRISPR/Cas or the CRISPR-Cas system, effects a cutting at a particular position; a specific DNA target. Accordingly, data can be generated—a data training set—relative to cutting by a CRISPR-Cas system at a particular position in a nucleotide, e.g., DNA, sequence at a particular position for a particular Cas or CRISPR enzyme. Similarly, data can be generated—a data training set—relative to cutting by a CRISPR-Cas system at a particular position in a nucleotide, e.g., DNA, sequence of a particular mismatch of typical nucleic acid hybridization (e.g., rather than G-C at particular position, G-T or G-U or G-A or G-G) for the particular Cas. In generating such data sets, there is the concept of average cutting frequency. The frequency by which an enzyme will cut a nucleic acid molecule, e.g., DNA, is mainly a function of the length of the sequence it is sensitive to. For instance, if an enzyme has a recognition sequence of 4 base-pairs, out of sheer probability, with 4 positions, and each position having potentially 4 different values, there are 4⁴ or 256 different possibilities for any given 4-base long strand. Therefore, theoretically (assuming completely random DNA), this enzyme will cut 1 in 256 4-base-pair long sites. For an enzyme that recognizes a sequence of 6 base-pairs, the calculation is 46 or 4096 possible combinations with this length, and so such an enzyme will cut 1 in 4096 6-base-pair long sites. Of course, such calculations take into consideration only that each position has potentially 4 different values, and completely random DNA. However, DNA is not completely random; for example, the G-C content of organisms varies. Accordingly, the data training set(s) in the invention come from observing cutting by a CRISPR-Cas system at a particular position in a nucleotide, e.g., DNA, sequence at a particular position for a particular Cas or CRISPR enzyme and observing cutting by a CRISPR-Cas system at a particular position in a nucleotide, e.g., DNA, sequence of a particular mismatch of typical nucleic acid hybridization for the particular Cas, in a statistically significant number of experiments as to the particular position, the CRISPR-Cas system and the particular Cas, and averaging the results observed or obtained therefrom. The average cutting frequency may be defined as the mean of the cleavage efficiencies for all guide RNA:target DNA mismatches at a particular location.

The invention further provides a method of identifying one or more unique target sequences, e.g., in a genome, such as a genome of a eukaryotic organism, whereby the target sequence is susceptible to being recognized by a CRISPR-Cas system (and likewise, the invention also further provides a method of identifying a CRISPR-Cas system susceptible to recognizing one or more unique target sequences), wherein the method comprises: a) determining average cutting frequency at a particular position for a particular Cas from a data training set as to that Cas, b) determining average cutting frequency of a particular mismatch (e.g., guide-RNA/target mismatch) for the particular Cas from the data training set, c) multiplying the average cutting frequency at a particular position by the average cutting frequency of a particular mismatch to obtain a first product, d) repeating steps a) to c) to obtain second and further products for any further particular position (s) of mismatches and particular mismatches and multiplying those second and further products by the first product, for an ultimate product, and omitting this step if there is no mismatch at any position or if there is only one particular mismatch at one particular position (or optionally d) repeating steps a) to c) to obtain second and further products for any further particular position (s) of mismatches and particular mismatches and multiplying those second and further products by the first product, for an ultimate product, and omitting this step if there is no mismatch at any position or if there is only one particular mismatch at one particular position), and e) multiplying the ultimate product by the result of dividing the minimum distance between consecutive mismatches by the distance, in bp, between the first and last base of the target sequence, e.g., 15-20, such as 18, and omitting this step if there is no mismatch at any position or if there is only one particular mismatch at one particular position (or optionally e) multiplying the ultimate product by the result of dividing the minimum distance between consecutive mismatches by the distance, in bp, between the first and last base of the target sequence, e.g., 15-20, such as 18 and omitting this step if there is no mismatch at any position or if there is only one particular mismatch at one particular position), to thereby obtain a ranking, which allows for the identification of one or more unique target sequences, to thereby obtain a ranking, which allows for the identification of one or more unique target sequences. Steps (a) and (b) can be performed in either order. If there are no other products than the first product, that first product (of step (c) from multiplying (a) times (b)) is what is used to determine or obtain the ranking.

The invention also comprehends method of identifying one or more unique target sequences in a genome of a eukaryotic organism, whereby the target sequence is susceptible to being recognized by a CRISPR-Cas system, wherein the method comprises: a) creating a data training set as to a particular Cas, b) determining average cutting frequency at a particular position for the particular Cas from the data training set, c) determining average cutting frequency of a particular mismatch for the particular Cas from the data training set, d) multiplying the average cutting frequency at a particular position by the average cutting frequency of a particular mismatch to obtain a first product, e) repeating steps b) to d) to obtain second and further products for any further particular position (s) of mismatches and particular mismatches and multiplying those second and further products by the first product, for an ultimate product, and omitting this step if there is no mismatch at any position or if there is only one particular mismatch at one particular position (or optionally e) repeating steps b) to d) to obtain second and further products for any further particular position (s) of mismatches and particular mismatches and multiplying those second and further products by the first product, for an ultimate product, and omitting this step if there is no mismatch at any position or if there is only one particular mismatch at one particular position), and f) multiplying the ultimate product by the result of dividing the minimum distance between consecutive mismatches by 18 and omitting this step if there is no mismatch at any position or if there is only one particular mismatch at one particular position (or optionally f) multiplying the ultimate product by the result of dividing the minimum distance between consecutive mismatches by the distance, in bp, between the first and last base of the target sequence, e.g., 15-20, such as 18, and omitting this step if there is no mismatch at any position or if there is only one particular mismatch at one particular position), to thereby obtain a ranking, which allows for the identification of one or more unique target sequences. Steps (a) and (b) can be performed in either order. Steps (a) and (b) can be performed in either order. If there are no other products than the first product, that first product (of step (c) from multiplying (a) times (b)) is what is used to determine or obtain the ranking.

The invention also comprehends a method of identifying one or more unique target sequences in a genome of a eukaryotic organism, whereby the target sequence is susceptible to being recognized by a CRISPR-Cas system, wherein the method comprises: a) determining average cutting frequency of guide-RNA/target mismatches at a particular position for a particular Cas from a training data set as to that Cas, and/or b) determining average cutting frequency of a particular mismatch-type for the particular Cas from the training data set, to thereby obtain a ranking, which allows for the identification of one or more unique target sequences. The method may comprise determining both the average cutting frequency of guide-RNA/target mismatches at a particular position for a particular Cas from a training data set as to that Cas, and the average cutting frequency of a particular mismatch-type for the particular Cas from the training data set. Where both are determined, the method may further comprise multiplying the average cutting frequency at a particular position by the average cutting frequency of a particular mismatch-type to obtain a first product, repeating the determining and multiplying steps to obtain second and further products for any further particular position(s) of mismatches and particular mismatches and multiplying those second and further products by the first product, for an ultimate product, and omitting this step if there is no mismatch at any position or if there is only one particular mismatch at one particular position, and multiplying the ultimate product by the result of dividing the minimum distance between consecutive mismatches by the distance, in bp, between the first and last base of the target sequence and omitting this step if there is no mismatch at any position or if there is only one particular mismatch at one particular position, to thereby obtain a ranking, which allows for the identification of one or more unique target sequences. The distance, in bp, between the first and last base of the target sequence may be 18. The method may comprise creating a training set as to a particular Cas. The method may comprise determining the average cutting frequency of guide-RNA/target mismatches at a particular position for a particular Cas from a training data set as to that Cas, if more than one mismatch, repeating the determining step so as to determine cutting frequency for each mismatch, and multiplying frequencies of mismatches to thereby obtain a ranking, which allows for the identification of one or more unique target sequences.

The invention further comprehends a method of identifying one or more unique target sequences in a genome of a eukaryotic organism, whereby the target sequence is susceptible to being recognized by a CRISPR-Cas system, wherein the method comprises: a) determining average cutting frequency of guide-RNA/target mismatches at a particular position for a particular Cas from a training data set as to that Cas, and average cutting frequency of a particular mismatch-type for the particular Cas from the training data set, to thereby obtain a ranking, which allows for the identification of one or more unique target sequences. The invention additionally comprehends a method of identifying one or more unique target sequences in a genome of a eukaryotic organism, whereby the target sequence is susceptible to being recognized by a CRISPR-Cas system, wherein the method comprises: a) creating a training data set as to a particular Cas, b) determining average cutting frequency of guide-RNA/target mismatches at a particular position for the particular Cas from the training data set, and/or c) determining average cutting frequency of a particular mismatch-type for the particular Cas from the training data set, to thereby obtain a ranking, which allows for the identification of one or more unique target sequences. The invention yet further comprehends a method of identifying one or more unique target sequences in a genome of a eukaryotic organism, whereby the target sequence is susceptible to being recognized by a CRISPR-Cas system, wherein the method comprises: a) creating a training data set as to a particular Cas, b) determining average cutting frequency of guide-RNA/target mismatches at a particular position for the particular Cas from the training data set, and average cutting frequency of a particular mismatch-type for the particular Cas from the training data set, to thereby obtain a ranking, which allows for the identification of one or more unique target sequences. Accordingly, in these embodiments, instead of multiplying cutting-frequency averages uniquely determined for a mismatch position and mismatch type separately, the invention uses averages that are uniquely determined, e.g., cutting-frequency averages for a particular mismatch type at a particular position (thereby without multiplying these, as part of preparation of training set). These methods can be performed iteratively akin to the steps in methods including multiplication, for determination of one or more unique target sequences.

The invention in certain aspects provides a method for selecting a CRISPR complex for targeting and/or cleavage of a candidate target nucleic acid sequence within a cell, comprising the steps of: (a) determining amount, location and nature of mismatch(es) of guide sequence of potential CRISPR complex(es) and the candidate target nucleic acid sequence, (b) determining contribution of each of the amount, location and nature of mismatch(es) to hybridization free energy of binding between the target nucleic acid sequence and the guide sequence of potential CRISPR complex(es) from a training data set, (c) based on the contribution analysis of step (b), predicting cleavage at the location(s) of the mismatch(es) of the target nucleic acid sequence by the potential CRISPR complex(es), and (d) selecting the CRISPR complex from potential CRISPR complex(es) based on whether the prediction of step (c) indicates that it is more likely than not that cleavage will occur at location(s) of mismatch(es) by the CRISPR complex Step (b) may be performed by: determining local thermodynamic contributions, ΔGᵢⱼ(k), between every guide sequence i and target nucleic acid sequence j at position k, wherein ΔGᵢⱼ(k) is estimated from a biochemical prediction algorithm and αₖ is a position-dependent weight calculated from the training data set, estimating values of the effective free-energy Zᵢⱼ using the relationship pᵢⱼ∝e^{-βz}^{ᵢⱼ}, wherein pᵢⱼ is measured cutting frequency by guide sequence i on target nucleic acid sequence j and β is a positive constant of proportionality, determining position-dependent weights αₖ by fitting across spacer/target-pairs with the sum across all N bases of the guide-sequence

$${Z}_{ij} = {\sum }_{k = 1}^{N} {α}_{k} Δ {G}_{ij} ( k )$$

and wherein, step (c) is performed by determining the position-dependent weights from the effective free-energy [symbol] = [symbol] between each spacer and every potential target in the genome, and determining estimated spacer-target cutting frequencies pₑₛₜ∝e^{-BZ}^{ₑₛₜ} to thereby predict cleavage. Beta is implicitly fit by fitting the values of alpha (that are completely free to be multiplied—in the process of fitting—by whichever constant is suitable for Z=sum(alpha*Delta G).

The invention also comprehends the creation of a training data set. A training data set is data of cutting frequency measurements, obtained to maximize coverage and redundancy for possible mismatch types and positions. There are advantageously two experimental paradigms for generating a training data set. In one aspect, generating a data set comprises assaying for Cas, e.g., Cas9, cleavage at a constant target and mutating guide sequences. In another aspect, generating a data set comprises assaying for Cas, e.g., Cas9, cleavage using a constant guide sequence and testing cleavage at multiple DNA targets. Further, the method can be performed in at least two ways: in vivo (in cells, tissue, or living animal) or in vitro (with a cell-free assay, using in vitro transcribed guide RNA and Cas, e.g., Cas9 protein delivered either by whole cell lysate or purified protein). Advantageously the method is performed by assaying for cleavage at a constant target with mismatched guide RNA in vivo in cell lines. Because the guide RNA may be generated in cells as a transcript from a RNA polymerase III promoter (e.g. U6) driving a DNA oligo, it may be expressed as a PCR cassette and transfect the guide RNA directly (FIG. 24c) along with CBh-driven Cas9 (PX165, FIG. 24c). By co-transfecting Cas9 and a guide RNA with one or several mismatches relative to the constant DNA target, one may assess cleavage at a constant endogenous locus by a nuclease assay such as SURVEYOR nuclease assay or next-generation deep sequencing. This data may be collected for at least one or multiple targets within a loci of interest, e.g., at least 1, at least 5, at least 10, at least 15 or at least 20 targets from the human EMX1 locus. In this manner, a data training set can be readily generated for any locus of interest. Accordingly, there are at least two ways for generating a data training set—in vivo (in cell lines or living animal) or in vitro (with a cell-free assay, using in vitro transcribed guide RNA and Cas, e.g., Cas9, protein delivered either by whole cell lysate or purified protein). Also, the experimental paradigm can differ—e.g. with mutated guide sequences or with a constant guide and an oligo library of many DNA targets. These targeting experiments can be done in vitro as well. The readout would simply be running a gel on the result of the in vitro cleavage assay—the results will be cleaved and uncleaved fractions. Alternatively or additionally, these fractions can be gel-isolated and sequencing adapters can be ligated prior to deep sequencing on these populations.

The invention comprehends computer-readable medium comprising codes that, upon execution by one or more processors, implements a herein method. The invention further comprehends a computer system for performing a herein method. The system can include I. a memory unit configured to receive and/or store sequence information of the genome; and II. one or more processors alone or in combination programmed to perform the herein method, whereby the identification of one or more unique target sequences is advantageously displayed or transmitted. The eukaryotic organism can be selected from the group consisting of Homo sapiens (human), Mus musculus (mouse), Rattus norvegicus (rat), Danio rerio (zebrafish), Drosophila melanogaster (fruit fly), Caenorhabditis elegans (roundworm), Sus scrofa (pig) and Bos taurus (cow). The target sequence can be a DNA sequence, and the mismatch(es) can be of RNA of the CRISPR complex and the DNA.

The invention also entails a method for selecting a CRISPR complex for targeting and/or cleavage of a candidate target nucleic acid sequence, e.g., within a cell, comprising the steps of: (a) determining amount, location and nature of mismatch(es) of potential CRISPR complex(es) and the candidate target nucleic acid sequence, (b) determining the contribution of the mismatch(es) based on the amount and location of the mismatch(es), (c) based on the contribution analysis of step (b), predicting cleavage at the location(s) of the mismatch(es), and (d) selecting the CRISPR complex from potential CRISPR complex(es) based on whether the prediction of step (c) indicates that it is more likely than not that cleavage will occur at location(s) of mismatch(es) by the CRISPR complex. The cell can be from a eukaryotic organism as herein discussed. The determining steps can be based on the results or data of the data training set(s) in the invention that come from observing cutting by a CRISPR-Cas system at a particular position in a nucleotide, e.g., DNA, sequence at a particular position for a particular Cas or CRISPR enzyme and observing cutting by a CRISPR-Cas system at a particular position in a nucleotide, e.g., DNA, sequence of a particular mismatch of typical nucleic acid hybridization for the particular Cas, in a statistically significant number of experiments as to the particular position, the CRISPR-Cas system and the particular Cas, and averaging the results observed or obtained therefrom. Accordingly, for example, if the data training set shows that at a particular position the CRISPR-Cas system including a particular Cas is rather promiscuous, i.e., there can be mismatches and cutting, the amount and location may be one position, and nature of the mismatch between the CRISPR complex and the candidate target nucleic acid sequence may be not serious such that the contribution of the mismatch to failure to cut/bind may be negligible and the prediction for cleavage may be more likely than not that cleavage will occur, despite the mismatch. Accordingly, it should be clear that the data training set(s) are not generated in silico but are generated in the laboratory, e.g., are from in vitro, ex vivo and/or in vivo studies. The results from the laboratory work, e.g., from in vitro, ex vivo and/or in vivo studies, are input into computer systems for performing herein methods.

In the herein methods the candidate target sequence can be a DNA sequence, and the mismatch(es) can be of RNA of potential CRISPR complex(es) and the DNA. In aspects of the invention mentioned herein, the amount of mismatches indicates the number of mismatches in DNA: RNA base pairing between the DNA of the target sequence and the RNA of the guide sequence. In aspects of the invention the location of mismatches indicates the specific location along the sequence occupied by the mismatch and if more than one mismatch is present if the mismatches are concatenated or occur consecutively or if they are separated by at least one of more residues. In aspects of the invention the nature of mismatches indicates the nucleotide type involved in the mismatched base pairing. Base pairs are matched according to G-C and A-U Watson-Crick base pairing.

The invention further involves a method for predicting the efficiency of cleavage at candidate target nucleic acid sequence, e.g., within a target in a cell, by a CRISPR complex comprising the steps of: (a) determining amount, location and nature of mismatch(es) of the CRISPR complex and the candidate target nucleic acid sequence, (b) determining the contribution of the mismatch(es) based on the amount and location of the mismatch(es), and (c) based on the contribution analysis of step (b), predicting whether cleavage is more likely than not to occur at location(s) of mismatch(es), and thereby predicting cleavage. As with other herein methods, the candidate target sequence can be a DNA sequence, and the mismatch(es) can be of RNA of the CRISPR complex and the DNA. The cell can be from a eukaryotic organism as herein discussed.

The invention even further provides a method for selecting a candidate target sequence, e.g., within a nucleic acid sequence, e.g., in a cell, for targeting by a CRISPR complex, comprising the steps of: determining the local thermodynamic contributions, ΔGᵢⱼ(k), between every spacer i and target j at position k, expressing an effective free-energy Zᵢⱼ for each spacer/target-pair as the sum

$${Z}_{ij} = {\sum }_{k = 1}^{N} {α}_{k} Δ {G}_{ij} ( k )$$

wherein ΔGᵢⱼ(k) is local thermodynamic contributions, estimated from a biochemical prediction algorithm and αₖ is position-dependent weights, and estimating the effective free-energy Z through the relationship pᵢⱼ∝e^{-βZ}^{ᵢⱼ} wherein pᵢⱼ is the measured cutting frequency by spacer i on target j and β is a positive constant fit across the entire data-set, and estimating the position-dependent weights αₖ by fitting [symbol] = [symbol] such that each spacer-target pair (i,j) corresponds to a row in the matrix (and each position k in the spacer-target pairing corresponds to a column in the same matrix, and estimating the effective free-energy [symbol] = [symbol] between each spacer and every potential target in the genome by using the fitted values ok, and selecting, based on calculated effective free-energy values, the candidate spacer/target pair ij according to their specificity and/or the efficiency, given the estimated spacer-target cutting frequencies pₑₛₜ∝e^{-βZ}^{ₑₛₜ}. The cell can be from a eukaryotic organism as herein discussed.

The invention includes a computer-readable medium comprising codes that, upon execution by one or more processors, implements a method for selecting a CRISPR complex for targeting and/or cleavage of a candidate target nucleic acid, e.g., sequence within a cell, comprising the steps of: (a) determining amount, location and nature of mismatch(es) of potential CRISPR complex(es) and the candidate target nucleic acid sequence, (b) determining the contribution of the mismatch(es) based on the amount and location of the mismatch(es), (c) based on the contribution analysis of step (b), predicting cleavage at the location(s) of the mismatch(es), and (d) selecting the CRISPR complex from potential CRISPR complex(es) based on whether the prediction of step (c) indicates that it is more likely than not that cleavage will occur at location(s) of mismatch(es) by the CRISPR complex. The cell can be from a eukaryotic organism as herein discussed.

Also, the invention involves computer systems for selecting a CRISPR complex for targeting and/or cleavage of a candidate target nucleic acid sequence, e.g., within a cell, the system comprising: a. a memory unit configured to receive and/or store sequence information of the candidate target nucleic acid sequence; and b. one or more processors alone or in combination programmed to (a) determine amount, location and nature of mismatch(es) of potential CRISPR complex(es) and the candidate target nucleic acid sequence, (b) determine the contribution of the mismatch(es) based on the amount and location of the mismatch(es), (c) based on the contribution analysis of step (b), predicting cleavage at the location(s) of the mismatch(es), and (d) select the CRISPR complex from potential CRISPR complex(es) based on whether the prediction of step (c) indicates that it is more likely than not that cleavage will occur at location(s) of mismatch(es) by the CRISPR complex. The cell can be from a eukaryotic organism as herein discussed. The system can display or transmit the selection.

In aspects of the invention mentioned herein, the amount of mismatches indicates the number of mismatches in DNA: RNA base pairing between the DNA of the target sequence and the RNA of the guide sequence. In aspects of the invention the location of mismatches indicates the specific location along the sequence occupied by the mismatch and if more than one mismatch is present if the mismatches are concatenated or occur consecutively or if they are separated by at least one of more residues. In aspects of the invention the nature of mismatches indicates the nucleotide type involved in the mismatched base pairing. Base pairs are matched according to G-C and A-U Watson-Crick base pairing.

Accordingly, aspects of the invention relate to methods and compositions used to determine the specificity of Cas9. In one aspect the position and number of mismatches in the guide RNA is tested against cleavage efficiency. This information enables the design of target sequences that have minimal off-target effects.

The invention also comprehends a method of identifying one or more unique target sequences in a genome of a eukaryotic organism, whereby the target sequence is susceptible to being recognized by a CRISPR-Cas system, wherein the method comprises a) determining average cutting frequency of guide-RNA/target mismatches at a particular position for a particular Cas from a training data set as to that Cas, and if more than one mismatch is present then step a) is repeated so as to determine cutting frequency for each mismatch after which frequencies of mismatches are multiplied to thereby obtain a ranking, which allows for the identification of one or more unique target sequences. The invention further comprehends a method of identifying one or more unique target sequences in a genome of a eukaryotic organism, whereby the target sequence is susceptible to being recognized by a CRISPR-Cas system, wherein the method comprises a) creating a training data set as to a particular Cas, b) determining average cutting frequency of guide-RNA/target mismatches at a particular position for a particular Cas from the training data set, if more than one mismatch exists, repeat step b) so as to determine cutting frequency for each mismatch, then multiply frequencies of mismatches to thereby obtain a ranking, which allows for the identification of one or more unique target sequences. The invention also relates to computer systems and computer readable media that executes these methods.

In various aspects, the invention involves a computer system for selecting a candidate target sequence within a nucleic acid sequence or for selecting a Cas for a candidate target sequence, e.g., selecting a target in a eukaryotic cell for targeting by a CRISPR complex.

The computer system may comprise: (a) a memory unit configured to receive and/or store said nucleic acid sequence; and (b) one or more processors alone or in combination programmed to perform as herein discussed. For example, programmed to: (i) locate a CRISPR motif sequence (e.g., PAM) within said nucleic acid sequence, and (ii) select a sequence adjacent to said located CRISPR motif sequence (e.g. PAM) as the candidate target sequence to which the CRISPR complex binds. In some embodiments, said locating step may comprise identifying a CRISPR motif sequence (e.g. PAM) located less than about 10000 nucleotides away from said target sequence, such as less than about 5000, 2500, 1000, 500, 250, 100, 50, 25, or fewer nucleotides away from the target sequence. In some embodiments, the candidate target sequence is at least 10, 15, 20, 25, 30, or more nucleotides in length. In some embodiments the candidate target sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, the nucleotide at the 3′ end of the candidate target sequence is located no more than about 10 nucleotides upstream of the CRISPR motif sequence (e.g. PAM), such as no more than 5, 4, 3, 2, or 1 nucleotides. In some embodiments, the nucleic acid sequence in the eukaryotic cell is endogenous to the cell or organism, e.g., eukaryotic genome. In some embodiments, the nucleic acid sequence in the eukaryotic cell is exogenous to the cell or organism, e.g., eukaryotic genome.

In various aspects, the invention provides a computer-readable medium comprising codes that, upon execution by one or more processors, implements a method described herein, e.g., of selecting a candidate target sequence within a nucleic acid sequence or selecting a CRISPR candidate for a target sequence; for instance, a target sequence in a cell such as in a eukaryotic cell for targeting by a CRISPR complex. The method can comprise: (i) locate a CRISPR motif sequence (e.g., PAM) within said nucleic acid sequence, and (ii) select a sequence adjacent to said located CRISPR motif sequence (e.g. PAM) as the candidate target sequence to which the CRISPR complex binds. In some embodiments, said locating step may comprise identifying a CRISPR motif sequence (e.g. PAM) located less than about 10000 nucleotides away from said target sequence, such as less than about 5000, 2500, 1000, 500, 250, 100, 50, 25, or fewer nucleotides away from the target sequence. In some embodiments, the candidate target sequence is at least 10, 15, 20, 25, 30, or more nucleotides in length. In some embodiments the candidate target sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, the nucleotide at the 3′ end of the candidate target sequence is located no more than about 10 nucleotides upstream of the CRISPR motif sequence (e.g. PAM), such as no more than 5, 4, 3, 2, or 1 nucleotides. In some embodiments, the nucleic acid sequence in the eukaryotic cell is endogenous to the cell or organism, e.g., eukaryotic genome. In some embodiments, the nucleic acid sequence in the eukaryotic cell is exogenous to the cell or organism, e.g., eukaryotic genome.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system comprises one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device comprising a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of RNA-guided Cas9 nuclease. The Cas9 nuclease from Streptococcus pyogenes is targeted to genomic DNA by a synthetic guide RNA (sgRNA) consisting of a 20-nt guide sequence and a scaffold. The guide sequence base-pairs with the DNA target, directly upstream of a requisite 5′-NGG protospacer adjacent motif (PAM; magenta), and Cas9 mediates a double-stranded break (DSB) ˜3 bp upstream of the PAM (indicated by triangle).

FIG. 2A-F. FIG. 2A shows an exemplary CRISPR system and a possible mechanism of action. FIG. 2B (left panel) provides an example adaptation of the CRISPR system for expression in eukaryotic cells, and also results of tests assessing nuclear localization and CRISPR activity (right panel). FIG. 2C illustrates mammalian expression of SpCas9 and SpRNase III driven by the constitutive EF1a promoter and tracrRNA and pre-crRNA array (DR-Spacer-DR) driven by the RNA Pol3 promoter U6; and discloses SEQ ID NOS 138-139, respectively, in order of appearance. FIG. 2D shows results of a surveyor nuclease assay for SpCas9-mediated insertions and deletions. FIG. 2E is a schematic representation of base pairing between target locus and EMX1-targeting crRNA, as well as an example chromatogram of a micro deletion adjacent to the SpCas9 cleavage site. FIG. 2E also discloses SEQ ID NOS 140-142, respectively, in order of appearance. FIG. 2F shows mutated alleles identified from sequencing analysis and discloses SEQ ID NOS 143-147, respectively, in order of appearance.

FIG. 3 shows a schematic representation assay carried out to evaluate the cleavage specificity of Cas9 form Streptococcus pyogenes. Single base pair mismatches between the guide RNA sequence and the target DNA are mapped against cleavage efficiency in %. FIG. 3 discloses SEQ ID NOS 148-149, respectively, in order of appearance.

FIG. 4 shows a mapping of mutations in the PAM sequence to cleavage efficiency in %.

FIG. 5A-C shows histograms of distances between adjacent S. pyogenes SF370 locus 1 PAM (NGG) (FIG. 5A) and S. thermophilus LMD9 locus 2 PAM (NNAGAAW) (FIG. 5B) in the human genome; and distances for each PAM by chromosome (Chr) (FIG. 5C).

FIG. 6A-C shows the graphing of distribution of distances between NGG (FIG. 6C) and NRG (FIG. 6A and FIG. 6B) motifs in the human genome in an “overlapping” and “non-overlapping” fashion.

FIG. 7A-D shows a circular depiction of the phylogenetic analysis revealing five families of Cas9s, including three groups of large Cas9s (˜1400 amino acids) and two of small Cas9s (˜1100 amino acids). FIG. 7A shows a first portion of the circular depiction. FIG. 7B shows a second portion of the circular depiction. FIG. 7C shows a third portion of the circular depiction. FIG. 7D shows a fourth portion of the circular depiction.

FIG. 8A shows one linear depiction of a phylogenetic analysis. FIG. 8B shows a second depiction of the phylogenetic analysis. FIG. 8C shows a third depiction of the phylogenetic analysis. FIG. 8D shows a fourth depiction of the phylogenetic analysis. FIG. 8E shows a fifth depiction of the phylogenetic analysis. FIG. 8F shows a sixth depiction of the phylogenetic analysis. The analyses reveal five families of Cas9s, including three groups of large Cas9s (˜1400 amino acids) and two of small Cas9s (˜1100 amino acids).

FIG. 9A-G shows the optimization of guide RNA architecture for SpCas9-mediated mammalian genome editing. FIG. 9A: Schematic of bicistronic expression vector (PX330) for U6 promoter-driven single guide RNA (sgRNA) and CBh promoter-driven human codon-optimized Streptococcus pyogenes Cas9 (hSpCas9) used for all subsequent experiments. The sgRNA consists of a 20-nt guide sequence (blue) and scaffold (red), truncated at various positions as indicated. FIG. 9A discloses SEQ ID NO: 150. FIG. 9B: SURVEYOR assay for SpCas9-mediated indels at the human EMX1 and PVALB loci. Arrows indicate the expected SURVEYOR fragments (n=3). FIG. 9C: Northern blot analysis for the four sgRNA truncation architectures, with U1 as loading control. FIG. 9D: Both wildtype (wt) or nickase mutant (D10A) of SpCas9 promoted insertion of a HindIII site into the human EMX1 gene. Single stranded oligonucleotides (ssODNs), oriented in either the sense or antisense direction relative to genome sequence, were used as homologous recombination templates (FIG. 68). FIG. 9E: Schematic of the human SERPINB5 locus. sgRNAs and PAMs are indicated by colored bars above sequence; methylcytosine (Me) are highlighted (pink) and numbered relative to the transcriptional start site (TSS, +1). FIG. 9E discloses SEQ ID NO: 151. FIG. 9F: Methylation status of SERPINB5 assayed by bisulfite sequencing of 16 clones. Filled circles, methylated CpG; open circles, unmethylated CpG. FIG. 9G: Modification efficiency by three sgRNAs targeting the methylated region of SERPINB5, assayed by deep sequencing (n=2). Error bars indicate Wilson intervals.

FIG. 10A-C shows position, distribution, number and mismatch-identity of some mismatch guide RNAs that can be used in generating the data training set (study on off target Cas9 activity). FIG. 10A discloses SEQ ID NOS 152-200, respectively, in order of appearance. FIG. 10B discloses SEQ ID NOS 201-249, respectively, in order of appearance. FIG. 10C discloses SEQ ID NOS 250-263, respectively, in order of appearance.

FIG. 11A-B shows further positions, distributions, numbers and mismatch-identities of some mismatch guide RNAs that can be used in generating the data training set (study on off target Cas9 activity). FIG. 11A and FIG. 11B form the first and second half of the Figure, respectively.

FIG. 12A-E shows guide RNA single mismatch cleavage efficiency. FIG. 12A: Multiple target sites were selected from the human EMX1 locus. Individual bases at positions 1-19 along the guide RNA sequence, which complementary to the target DNA sequence, were mutated to every ribonucleotide mismatch from the original guide RNA (blue ‘N’). FIG. 12A discloses SEQ ID NOS 264-284, respectively, in order of appearance. FIG. 12B: On-target Cas9 cleavage activity for guide RNAs containing single base mutations (light blue: high cutting, dark blue: low cutting) relative to the on-target guide RNA (grey). FIG. 12B discloses SEQ ID NOS 285-287, respectively, in order of appearance. FIG. 12C: Base transition heat map representing relative Cas9 cleavage activity for each possible RNA:DNA base pair. Rows were sorted based on cleavage activity in the PAM-proximal 10 bases of the guide RNA (high to low). Mean cleavage levels were calculated across base transitions in the PAM-proximal 10 bases (right bar) and across all transitions at each position (bottom bar). Heat map represents aggregate single-base mutation data from 15 EMX1 targets. FIG. 12D: Mean Cas9 locus modification efficiency at targets with all possible PAM sequences. FIG. 12E: Histogram of distances between 5′-NRG PAM occurrences within the human genome. Putative targets were identified using both the plus and minus strand of human chromosomal sequences.

FIG. 13A-C shows Cas9 on-target cleavage efficiency with multiple guide RNA mismatches and genome-wide specificity. a, Cas9 targeting efficiency with guide RNAs containing concatenated mismatches of 2 (top), 3 (middle), or 5 (bottom) consecutive bases for EMX1 targets 1 and 6. Rows represent different mutated guide RNAs and show the identity of each nucleotide mutation (white cells; grey cells denote unmutated bases). FIG. 13A discloses SEQ ID NOS 288-310 in the first block of alignments and SEQ ID NOS 311-333 in the second block alignments, respectively, in order of appearance. b, Cas9 was targeted with guide RNAs containing 3 (top, middle) or 4 (bottom) mismatches (white cells) separated by different numbers of unmutated bases (gray cells). FIG. 13B discloses SEQ ID NOS 334-353 in the first block of alignments and SEQ ID NOS 354-373 in the second block of alignments, respectively, in order of appearance. c, Cleavage activity at targeted EMX1 target loci (top bar) as well as at candidate off-target genomic sites. Putative off-target loci contained 1-3 individual base differences (white cells) compared to the on-target loci. FIG. 13C discloses SEQ ID NOS 374-427, respectively, in order of appearance.

FIG. 14A-B shows SpCas9 cleaves methylated targets in vitro. FIG. 14A: Plasmid targets containing CpG dinucleotides are either left unmethylated or methylated in vitro by M.SssI. Methyl-CpG in either the target sequence or PAM are indicated. FIG. 14A discloses SEQ ID NOS 428, 428-429 and 429, respectively, in order of appearance. FIG. 14B: Cleavage of either unmethylated or methylated targets 1 and 2 by SpCas9 cell lysate.

FIG. 15 shows a UCSC Genome Browser track for identifying unique S. pyogenes Cas9 target sites in the human genome. A list of unique sites for the human, mouse, rat, zebrafish, fruit fly, and C. elegans genomes have been computationally identified and converted into tracks that can be visualized using the UCSC genome browser. Unique sites are defined as those sites with seed sequences (3′-most 12 nucleotides of the spacer sequence plus the NGG PAM sequence) that are unique in the entire genome. FIG. 15 discloses SEQ ID NOS 430-508, respectively, in order of appearance.

FIG. 16 shows a UCSC Genome Browser track for identifying unique S. pyogenes Cas9 target sites in the mouse genome. FIG. 16 discloses SEQ ID NOS 509-511, respectively, in order of appearance.

FIG. 17 shows a UCSC Genome Browser track for identifying unique S. pyogenes Cas9 target sites in the rat genome. FIG. 17 discloses SEQ ID NOS 512-552, respectively, in order of appearance.

FIG. 18 shows a UCSC Genome Browser track for identifying unique S. pyogenes Cas9 target sites in the zebra fish genome. FIG. 18 discloses SEQ ID NOS 553-570, respectively, in order of appearance.

FIG. 19 shows a UCSC Genome Browser track for identifying unique S. pyogenes Cas9 target sites in the D. melanogaster genome. FIG. 19 discloses SEQ ID NOS 571-662, respectively, in order of appearance.

FIG. 20 shows a UCSC Genome Browser track for identifying unique S. pyogenes Cas9 target sites in the (′. elegans genome. FIG. 20 discloses SEQ ID NOS 663-708, respectively, in order of appearance.

FIG. 21 shows a UCSC Genome Browser track for identifying unique S. pyogenes Cas9 target sites in the pig genome. FIG. 21 discloses SEQ ID NOS 709-726, 1076, 727-743, respectively, in order of appearance.

FIG. 22 shows a UCSC Genome Browser track for identifying unique S. pyogenes Cas9 target sites in the cow genome. FIG. 22 discloses SEQ ID NO: 744.

FIG. 23 shows CRISPR Designer, a web app for the identification of Cas9 target sites. Most target regions (such as exons) contain multiple possible CRISPR sgRNA+PAM sequences. To minimize predicted off-targeted cleavage across the genome, a web-based computational pipeline ranks all possible sgRNA sites by their predicted genome-wide specificity and generates primers and oligos required for construction of each possible CRISPR as well as primers (via Primer3) for high-throughput assay of potential off-target cleavage in a next-generation sequencing experiment. Optimization of the choice of sgRNA within a user's target sequence: The goal is to minimize total off-target activity across the human genome. For each possible sgRNA choice, there is identification of off-target sequences (preceding either NAG or NGG PAMs) across the human genome that contain up to 5 mismatched base-pairs. The cleavage efficiency at each off-target sequence is predicted using an experimentally-derived weighting scheme. Each possible sgRNA is then ranked according to its total predicted off-target cleavage; the top-ranked sgRNAs represent those that are likely to have the greatest on-target and the least off-target cleavage. In addition, automated reagent design for CRISPR construction, primer design for the on-target SURVEYOR assay, and primer design for high-throughput detection and quantification of off-target cleavage via next-gen sequencing are advantageously facilitated. FIG. 23 discloses SEQ ID NOS 128 and 745-761, respectively, in order of appearance.

FIG. 24A-C shows Target selection and reagent preparation. FIG. 24A: For S. pyogenes Cas9, 20-bp targets (highlighted in blue) must be followed by 5′-NGG, which can occur in either strand on genomic DNA. FIG. 24B: Schematic for co-transfection of Cas9 expression plasmid (PX165) and PCR-amplified U6-driven sgRNA expression cassette. Using a U6 promoter-containing PCR template and a fixed forward primer (U6 Fwd), sgRNA-encoding DNA can appended onto the U6 reverse primer (U6 Rev) and synthesized as an extended DNA oligo (Ultramer oligos from IDT). Note the guide sequence (blue N's) in U6 Rev is the reverse complement of the 5′-NGG flanking target sequence. FIG. 24B discloses SEQ ID NOS 762-765, respectively, in order of appearance. FIG. 24C: Schematic for scarless cloning of the guide sequence oligos into a plasmid containing Cas9 and sgRNA scaffold (PX330). The guide oligos (blue N's) contain overhangs for ligation into the pair of BbsI sites on PS330, with the top and bottom strand orientations matching those of the genomic target (i.e. top oligo is the 20-bp sequence preceding 5′-NGG in genomic DNA). Digestion of PX330 with BbsI allows the replacement of the Type IIs restriction sites (blue outline) with direct insertion of annealed oligos. It is worth noting that an extra G was placed before the first base of the guide sequence. Applicants have found that an extra G in front of the guide sequence does not adversely affect targeting efficiency. In cases when the 20-nt guide sequence of choice does not begin with guanine, the extra guanine will ensure the sgRNA is efficiently transcribed by the U6 promoter, which prefers a guanine in the first base of the transcript. FIG. 24C discloses SEQ ID NOS 766-768, respectively, in order of appearance.

FIG. 25A-E shows the single nucleotide specificity of SpCas9. FIG. 25A: Schematic of the experimental design. sgRNAs carrying all possible single base-pair mismatches (blue Ns) throughout the guide sequence were tested for each EMX1 target site (target site 1 shown as example). FIG. 25A discloses SEQ ID NOS 264-284, respectively, in order of appearance. FIG. 24B: Heatmap representation of relative SpCas9 cleavage efficiency by 57 single-mutated and 1 non-mutated sgRNA s each for four EMX1 target sites. For each EMX1 target, the identities of single base-pair substitutions are indicated on the left; original guide sequence is shown above and highlighted in the heatmap (grey squares). Modification efficiencies (increasing from white to dark blue) are normalized to the original guide sequence. FIG. 25B discloses SEQ ID NOS 285-286, 769 and 287, respectively, in order of appearance. FIG. 25C: Heatmap for relative SpCas9 cleavage efficiency for each possible RNA:DNA base pair, compiled from aggregate data from single-mismatch guide RNAs for 15 EMX1 targets. Mean cleavage levels were calculated for the 10 PAM-proximal bases (right bar) and across all substitutions at each position (bottom bar); positions in grey were not covered by the 469 single-mutated and 15 non-mutated sgRNAs tested. FIG. 25D: SpCas9-mediated indel frequencies at targets with all possible PAM sequences, determined using the SURVEYOR nuclease assay. Two target sites from the EMX1 locus were tested for each PAM (Table 4). FIG. 25E: Histogram of distances between 5′-NRG PAM occurrences within the human genome. Putative targets were identified using both strands of human chromosomal sequences (GRCh37/hg19).

FIG. 26A-C shows the multiple mismatch specificity of SpCas9. (a) SpCas9 cleavage efficiency with guide RNAs containing a, consecutive mismatches of 2, 3, or 5 bases, or (b, c) multiple mismatches separated by different numbers of unmutated bases for EMX1 targets 1, 2, 3, and 6. Rows represent each mutated guide RNA; nucleotide substitutions are shown in white cells; grey cells denote unmutated bases. All indel frequencies are absolute and analyzed by deep sequencing from 2 biological replicas. Error bars indicate Wilson intervals (Example 7, Methods and Materials). FIG. 26A discloses SEQ ID NOS 770-790 as the “target 1” sequences, SEQ ID NOS 791-811 as the “target 2” sequences, SEQ ID NOS 812-832 as the “target 3” sequences and SEQ ID NOS 833-853 as the “target 6” sequences, all respectively, in order of appearance. FIG. 26B discloses SEQ ID NOS 854-867 as the “target 1” sequences, SEQ ID NOS 868-881 as the “target 2” sequences, SEQ ID NOS 882-895 as the “target 3” sequences and SEQ ID NOS 896-909 as the “target 6” sequences, all respectively, in order of appearance. FIG. 26C discloses SEQ ID NOS 910-923 as the “target 1” sequences, SEQ ID NOS 924-937 as the “target 2” sequences, SEQ ID NOS 938-951 as the “target 3” sequences and SEQ ID NOS 952-965 as the “target 6” sequences, all respectively, in order of appearance.

FIG. 27A-D shows SpCas9-mediated indel frequencies at predicted genomic off-target loci. Cleavage levels at putative genomic off-target loci containing 2 or 3 individual mismatches (white cells) for EMX1 target 1 (FIG. 27A) and target 3 (FIG. 27B) are analyzed by deep sequencing. List of off-target sites are ordered by median position of mutations. Putative off-target sites with additional mutations did not exhibit detectable indels (Table 4). The Cas9 dosage was 3×10−10 nmol/cell, with equimolar sgRNA delivery. Error bars indicate Wilson intervals. Indel frequencies for EMX1 targets 1 (FIG. 27C) and 3 (FIG. 27D) and selected off target loci (OT) as a function of SpCas9 and sgRNA dosage, normalized to on-target cleavage at highest transfection dosage (n=2). 400 ng to 10 ng of Cas9-sgRNA plasmid corresponds to 7.1×10−10 to 1.8×10−11 nmol/cell. Cleavage specificity is measured as a ratio of on- to off-target cleavage. FIG. 27A discloses the “target 1” sequences as SEQ ID NOS 966-975 and the “locus target” sequences as SEQ ID NOS 976-983, respectively, in order of appearance. FIG. 27B discloses the “target 3” sequences as SEQ ID NOS 984-1017 and the “locus target” sequences as SEQ ID NOS 1018-1039, respectively, in order of appearance.

FIG. 28A-B shows the human EMX1 locus with target sites. Schematic of the human EMX1 locus showing the location of 15 target DNA sites, indicated by blue lines with corresponding PAM in magenta. FIG. 28A discloses SEQ ID NO: 1040. FIG. 28B discloses SEQ ID NOS 1041-1055, respectively, in order of appearance.

FIG. 29A-B shows additional genomic off-target site analysis. Cleavage levels at candidate genomic off-target loci (white cells) for a, EMX1 target 2 and b, EMX1 target 6 were analyzed by deep sequencing. All indel frequencies are absolute and analyzed by deep sequencing from 2 biological replicates. Error bars indicate Wilson confidence intervals. FIG. 29A discloses SEQ ID NOS 1056-1062, respectively, in order of appearance. FIG. 29B discloses SEQ ID NOS 1063-1065, respectively, in order of appearance.

FIG. 30 shows predicted and observed cutting frequency-ranks among genome-wide targets.

FIG. 31 shows that the PAM for Staphylococcus aureus sp. Aureus Cas9 is NNGRR. FIG. 31 discloses SEQ ID NOS 1066-1075, respectively, in order of appearance.

FIG. 32 shows a flow diagram as to locational methods of the invention.

FIG. 33A-B shows a first (FIG. 33A) and a second (FIG. 33B) flow diagram as to thermodynamic methods of the invention.

FIG. 34 shows a flow diagram as to multiplication methods of the invention.

FIG. 35 shows a schematic block diagram of a computer system which can be used to implement the methods described herein.

The figures herein are for illustrative purposes only and are not necessarily drawn to scale.

### DETAILED DESCRIPTION OF THE INVENTION

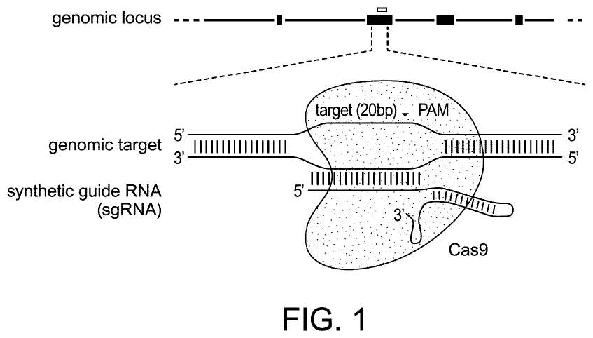

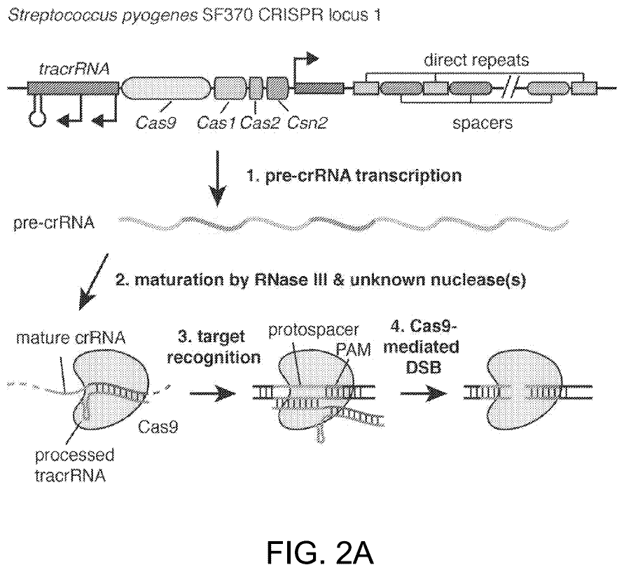

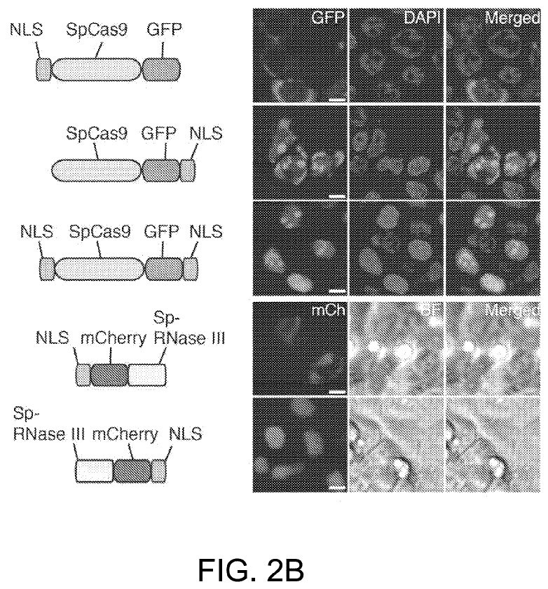

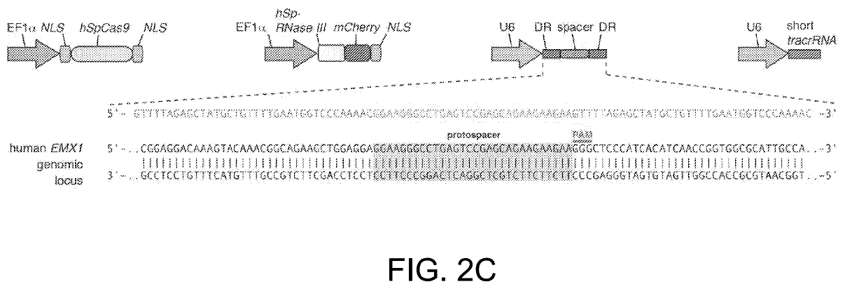

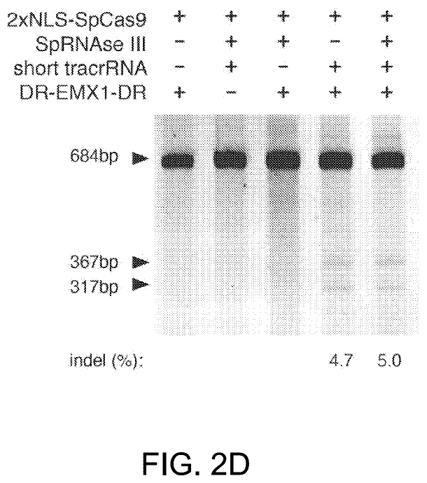

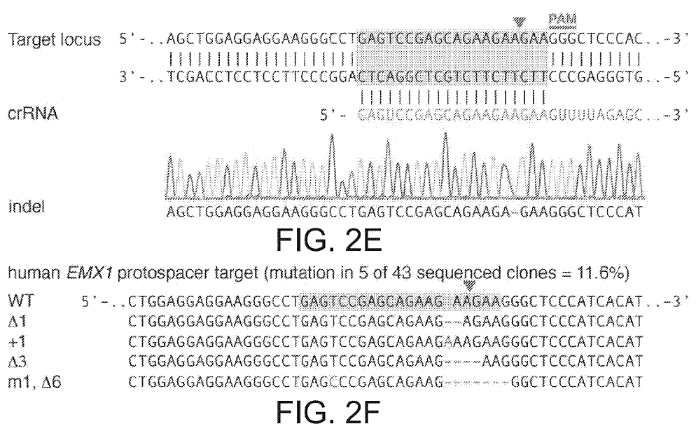

The invention relates to the engineering and optimization of systems, methods and compositions used for the control of gene expression involving sequence targeting, such as genome perturbation or gene-editing, that relate to the CRISPR/Cas system and components thereof (FIGS. 1 and 2). In advantageous embodiments, the Cas enzyme is Cas9.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “domain” or “protein domain” refers to a part of a protein sequence that may exist and function independently of the rest of the protein chain.

As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the dTALEs described herein have sequences that are at least 95% identical or share identity to the capping region amino acid sequences provided herein.

Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program. % homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible—reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension. Calculation of maximum % homology therefore first requires the production of an optimal alignment, taking into consideration gap penalties. A suitable computer program for carrying out such an alignment is the GCG Wisconsin Bestfit package (Devereux et al., 1984 Nuc. Acids Research 12 p387). Examples of other software than may perform sequence comparisons include, but are not limited to, the BLAST package (see Ausubel et al., 1999 Short Protocols in Molecular Biology, 4th Ed.—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins DG & Sharp PM (1988), Gene 73(1), 237-244). Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. However, it is more useful to include mutation data as well. The sets of amino acids thus derived are likely to be conserved for structural reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids.

| Set | | Sub-set | |
|---|---|---|---|
| Hydrophobic | F W Y H K M I L V A G C | Aromatic | F W Y H |
| | | Aliphatic | I L V |
| Polar | W Y H K R E D C S T N Q | Charged | H K R E D |
| | | Positively | H K R |
| | | charged | |
| | | Negatively | E D |
| | | charged | |
| Small | V C A G S P T N D | Tiny | A G S |

Embodiments of the invention include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids such as basic for basic, acidic for acidic, polar for polar, etc. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Variant amino acid sequences may include suitable spacer groups that may be inserted between any two amino acid residues of the sequence including alkyl groups such as methyl, ethyl or propyl groups in addition to amino acid spacers such as glycine or ß-alanine residues. A further form of variation, which involves the presence of one or more amino acid residues in peptoid form, may be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

In one aspect, the invention provides for vectors that are used in the engineering and optimization of CRISPR/Cas systems. A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, the contents of which are herein incorporated by reference in their entirety.

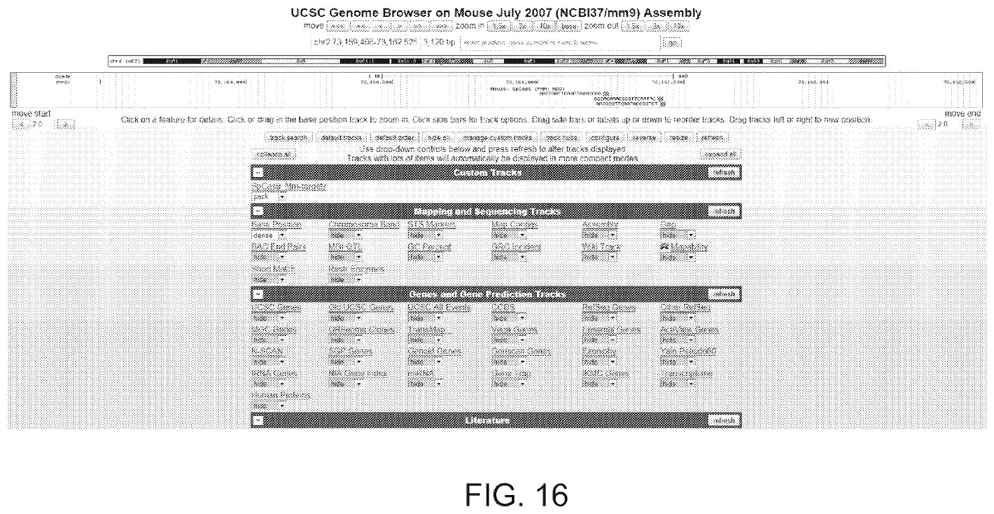

Aspects of the invention can relate to bicistronic vectors for chimeric RNA and Cas9. Cas9 is driven by the CBh promoter and the chimeric RNA is driven by a U6 promoter. The chimeric guide RNA consists of a 20 bp guide sequence (Ns) joined to the tracr sequence (running from the first “U” of the lower strand to the end of the transcript), which is truncated at various positions as indicated. The guide and tracr sequences are separated by the tracr-mate sequence GUUUUAGAGCUA (SEQ ID NO: 1) followed by the loop sequence GAAA. Results of SURVEYOR assays for Cas9-mediated indels at the human EMX1 and PVALB loci are illustrated in FIGS. 16b and 16c, respectively. Arrows indicate the expected SURVEYOR fragments. ChiRNAs are indicated by their “+n” designation, and crRNA refers to a hybrid RNA where guide and tracr sequences are expressed as separate transcripts. Throughout this application, chimeric RNA (chiRNA) may also be called single guide, or synthetic guide RNA (sgRNA).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). With regards to regulatory sequences, mention is made of U.S. patent application Ser. No. 10/491,026, the contents of which are incorporated by reference herein in their entirety. With regards to promoters, mention is made of PCT publication WO 2011/028929 and U.S. application Ser. No. 12/511,940, the contents of which are incorporated by reference herein in their entirety.

Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase. Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). In some embodiments, a vector is a yeast expression vector. Examples of vectors for expression in yeast Saccharomyces cerivisae include pYepSecl (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (In Vitrogen Corp, San Diego, Calif.). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regard to these prokaryotic and eukaryotic vectors, mention is made of U.S. Pat. No. 6,750,059, the contents of which are incorporated by reference herein in their entirety. Other embodiments of the invention may relate to the use of viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety.

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In embodiments of the invention the terms guide sequence and guide RNA are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

In preferred embodiments of the invention, the CRISPR system is a type II CRISPR system and the Cas enzyme is Cas9, which catalyzes DNA cleavage. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence NGG following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archae, Mole Cell 2010, January 15; 37(1): 7.

The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. Several aspects of the CRISPR system can be further improved to increase the efficiency and versatility of CRISPR targeting. Optimal Cas9 activity may depend on the availability of free Mg2+ at levels higher than that present in the mammalian nucleus (see e.g. Jinek et al., 2012, Science, 337:816), and the preference for an NGG motif immediately downstream of the protospacer restricts the ability to target on average every 12-bp in the human genome.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III or the HNH domain) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. An aspartate-to-alanine substitution (D10A) in the RuvC I catalytic domain of SpCas9 converts the nuclease into a nickase (see e.g. Sapranauskas et al., 2011, Nucleic Acis Research, 39: 9275; Gasiunas et al., 2012, Proc. Natl. Acad. Sci. USA, 109:E2579), such that nicked genomic DNA undergoes the high-fidelity homology-directed repair (HDR). In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, See Nakamura, Y., et al. “Codon usage tabulated from the international DNA sequence databases: status for the year 2000”Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In some embodiments, a vector encodes a CRISPR enzyme comprising one or more nuclear localization sequences (NLSs), such as about or more than about 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the CRISPR enzyme comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 2); the NLS from nucleoplasmin (e.g. the nucleoplasmin bipartite NLS with the sequence KRPAATKKAGQAKKKK (SEQ ID NO: 3)); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence P[[O]]QPKKKPL (SEQ ID NO: 10) of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS1; the sequence RKLKKKIKKL (SEQ ID NO: 14) of the Hepatitis virus delta antigen; the sequence REKKKFLKRR (SEQ ID NO: 15) of the mouse Mx1 protein; the sequence KRKGDEVDGVDEVAKKKSKK (SEQ ID NO: 16) of the human poly(ADP-ribose) polymerase; and the sequence RKCLQAGMNLEARKTKK (SEQ ID NO: 17) of the steroid hormone receptors (human) glucocorticoid.

In general, the one or more NLSs are of sufficient strength to drive accumulation of the CRISPR enzyme in a detectable amount in the nucleus of a eukaryotic cell. In general, strength of nuclear localization activity may derive from the number of nuclear localization sequence(s) (NLS(s)) in the CRISPR enzyme, the particular NLS(s) used, or a combination of these factors. Detection of accumulation in the nucleus may be performed by any suitable technique. For example, a detectable marker may be fused to the CRISPR enzyme, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

In general, a guide sequence is any polynucleotide sequence having sufficient complementarity with a target polynucleotide sequence to hybridize with the target sequence and direct sequence-specific binding of a CRISPR complex to the target sequence. Throughout this application the guide sequence may be interchangeably referred to as a guide or a spacer. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by SURVEYOR assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything) has a single occurrence in the genome. A unique target sequence in a genome may include an S. pyogenes Cas9 target site of the form MMMMMMMMMNNNNNNNNNNNXGG where NNNNNNNNNNNXGG (N is A, G, T, or C; and X can be anything) has a single occurrence in the genome. For the S. thermophilus CRISPR1 Cas9, a unique target sequence in a genome may include a Cas9 target site of the form MMMMMMMMNNNNNNNNNNNNXXAGAAW (SEQ ID NO: 18) where NNNNNNNNNNNNXXAGAAW (SEQ ID NO: 19) (N is A, G, T, or C; X can be anything; and W is A or T) has a single occurrence in the genome. A unique target sequence in a genome may include S. thermophilus site the form an CRISPR1 Cas9 target MMMMMMMMMNNNNNNNNNNNXXAGAAW (SEQ ID NO: 20) where NNNNNNNNNNNXXAGAAW (SEQ ID NO: 21) (N is A, G, T, or C; X can be anything; and W is A or T) has a single occurrence in the genome. For the S. pyogenes Cas9, a unique target sequence in a genome may include a Cas9 target site of the form MMMMMMMMNNNNNNNNNNNNXGGXG where NNNNNNNNNNNNXGGXG (N is A, G, T, or C; and X can be anything) has a single occurrence in the genome. A unique target sequence in a genome may include an S. pyogenes Cas9 target site of the form MMMMMMMMMNNNNNNNNNNNXGGXG where NNNNNNNNNNNXGGXG (N is A, G, T, or C; and X can be anything) has a single occurrence in the genome. In each of these sequences “M” may be A, G, T, or C, and need not be considered in identifying a sequence as unique.

In some embodiments, a guide sequence is selected to reduce the degree secondary structure within the guide sequence. In some embodiments, about or less than about 75%, 50%, 40%, 30%, 25%, 20%, 15%, 10%, 5%, 1%, or fewer of the nucleotides of the guide sequence participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62).

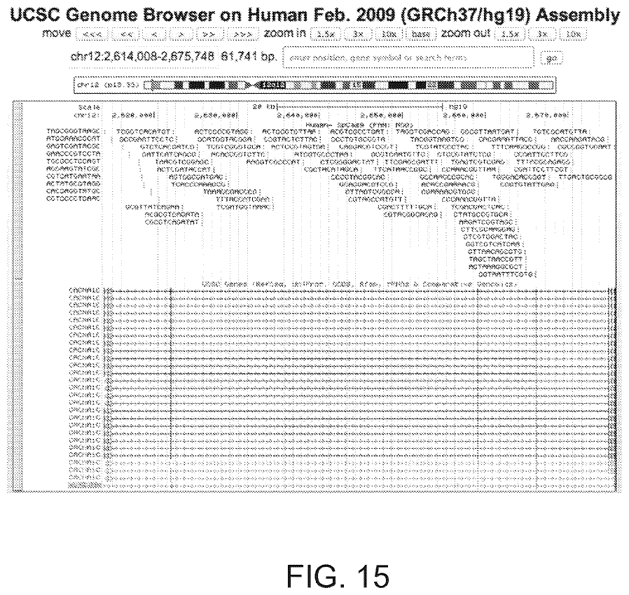

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence An example illustration of such a hairpin structure is provided in the lower portion of FIG. 15B. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator: (1) NNNNNNNNNNNNNNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 22); (2) NNNNNNNNNNNNNNNNNNNNgtttttgtactctcaGAAAtgcagaagctacaaagataaggcttcatgccgaaatca acaccctgtcattttatggcagggtgttttcgttatttaaTTTTTT (SEQ ID NO: 23); (3) NNNNNNNNNNNNNNNNNNNNgtttttgtactctcaGAAAtgcagaagctacaaagataaggcttcatgccgaaatca acaccctgtcattttatggcagggtgtTTTTTT (SEQ ID NO: 24); (4) NNNNNNNNNNNNNNNNNNNNgttttagagctaGAAAtagcaagttaaaataaggctagtccgttatcaacttgaaaa agtggcaccgagtcggtgcTTTTTT (SEQ ID NO: 25); (5) NNNNNNNNNNNNNNNNNNNNgttttagagctaGAAATAGcaagttaaaataaggctagtccgttatcaacttgaa aaagtgTTTTTTT (SEQ ID NO: 26); and (6) NNNNNNNNNNNNNNNNNNNNgttttagagctagAAATAGcaagttaaaataaggctagtccgttatcaTTTTT TTT (SEQ ID NO: 27). In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, a recombination template is also provided. A recombination template may be a component of another vector as described herein, contained in a separate vector, or provided as a separate polynucleotide. In some embodiments, a recombination template is designed to serve as a template in homologous recombination, such as within or near a target sequence nicked or cleaved by a CRISPR enzyme as a part of a CRISPR complex. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In some embodiments, a CRISPR enzyme may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc), or light inducible systems (Phytochrome, LOV domains, or cryptochorome). In one embodiment, the CRISPR enzyme may be a part of a Light Inducible Transcriptional Effector (LITE) to direct changes in transcriptional activity in a sequence-specific manner. The components of a light may include a CRISPR enzyme, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, which is hereby incorporated by reference in its entirety.

In some aspects, the invention comprehends delivering one or more polynucleotides, such as or one or more vectors as described herein, one or more transcripts thereof, and/or one or proteins transcribed therefrom, to a host cell. In some aspects, the invention comprehends cells produced by such methods, and animals comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

In some embodiments, a host cell contains the target sequence, and the cell can be derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CVI, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP 1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds. Target sequence(s) can be in such cells.

With recent advances in crop genomics, the ability to use CRISPR-Cas9 systems to perform efficient and cost effective gene editing and manipulation will allow the rapid selection and comparison of single and multiplexed genetic manipulations to transform such genomes for improved production and enhanced traits. In this regard reference is made to US patents and publications: U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, all the contents and disclosure of each of which are herein incorporated by reference in their entirety. In the practice of the invention, the contents and disclosure of Morrell et al “Crop genomics:advances and applications” Nat Rev Genet. 2011 Dec. 29; 13(2):85-96 are also herein incorporated by reference in their entirety. In an advantageous embodiment of the invention, the CRISPR/Cas9 system is used to engineer microalgae. Thus, target polynucleotides in the invention can be plant, algae, prokaryotic or eukaryotic.

CRISPR systems can be useful for creating an animal or cell that may be used as a disease model. Thus, identification of target sequences for CRISPR systems can be useful for creating an animal or cell that may be used as a disease model. As used herein, “disease” refers to a disease, disorder, or indication in a subject. For example, a method of the invention may be used to create an animal or cell that comprises a modification in one or more nucleic acid sequences associated with a disease, or an animal or cell in which the expression of one or more nucleic acid sequences associated with a disease are altered. Such a nucleic acid sequence may encode a disease associated protein sequence or may be a disease associated control sequence.

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

CRISPR systems can be used to develop a biologically active agent that modulates a cell signaling event associated with a disease gene; and hence, identifying target sequences can be so used.

CRISPR systems can be used to develop a cell model or animal model can be constructed in combination with the method of the invention for screening a cellular function change; and hence, identifying target sequences can be so used. Such a model may be used to study the effects of a genome sequence modified by the CRISPR complex of the invention on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

An altered expression of one or more genome sequences associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product. To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the ac companying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, ß-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphorimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent:protein complex so formed. In one aspect of this embodiment, the agent that specifically binds a protein associated with a signaling biochemical pathway is an antibody, preferably a monoclonal antibody. The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent:polypeptide complex. However, the label is typically designed to be accessible to an antibody for an effective binding and hence generating a detectable signal. A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Anti-phosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2α). Alternatively, these antibodies can be generated using conventional polyclonal or monoclonal antibody technologies by immunizing a host animal or an antibody-producing cell with a target protein that exhibits the desired post-translational modification.

It may be desirable to discern the expression pattern of an protein associated with a signaling biochemical pathway in different bodily tissue, in different cell types, and/or in different subcellular structures. These studies can be performed with the use of tissue-specific, cell-specific or subcellular structure specific antibodies capable of binding to protein markers that are preferentially expressed in certain tissues, cell types, or subcellular structures.

An altered expression of a gene associated with a signaling biochemical pathway can also be determined by examining a change in activity of the gene product relative to a control cell. The assay for an agent-induced change in the activity of a protein associated with a signaling biochemical pathway will dependent on the biological activity and/or the signal transduction pathway that is under investigation. For example, where the protein is a kinase, a change in its ability to phosphorylate the downstream substrate(s) can be determined by a variety of assays known in the art. Representative assays include but are not limited to immunoblotting and immunoprecipitation with antibodies such as anti-phosphotyrosine antibodies that recognize phosphorylated proteins. In addition, kinase activity can be detected by high throughput chemiluminescent assays such as AlphaScreen™ (available from Perkin Elmer) and eTag™ assay (Chan-Hui, et al. (2003) Clinical Immunology 111: 162-174).

Where the protein associated with a signaling biochemical pathway is part of a signaling cascade leading to a fluctuation of intracellular pH condition, pH sensitive molecules such as fluorescent pH dyes can be used as the reporter molecules. In another example where the protein associated with a signaling biochemical pathway is an ion channel, fluctuations in membrane potential and/or intracellular ion concentration can be monitored. A number of commercial kits and high-throughput devices are particularly suited for a rapid and robust screening for modulators of ion channels. Representative instruments include FLIPR™ (Molecular Devices, Inc.) and VIPR (Aurora Biosciences). These instruments are capable of detecting reactions in over 1000 sample wells of a microplate simultaneously, and providing real-time measurement and functional data within a second or even a minisecond.

In practicing any of the methods disclosed herein, a suitable vector can be introduced to a cell or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA).

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA).

The target polynucleotide of a CRISPR complex may include a number of disease-associated genes and polynucleotides as well as signaling biochemical pathway-associated genes and polynucleotides as listed in U.S. provisional patent applications 61/736,527 and 61/748,427 having Broad reference BI-2011/008/WSGR Docket No. 44063-701.101 and BI-2011/008/WSGR Docket No. 44063-701.102 respectively, both entitled SYSTEMS METHODS AND COMPOSITIONS FOR SEQUENCE MANIPULATION filed on Dec. 12, 2012 and Jan. 2, 2013, respectively, the contents of all of which are herein incorporated by reference in their entirety.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Embodiments of the invention also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011—Medical). Specific aspects of tandem repeat sequences have been found to be responsible for more than twenty human diseases (New insights into repeat instability: role of RNA.DNA hybrids. Mclvor E I, Polak U, Napierala M. RNA Biol. 2010 September-October; 7(5):551-8). The CRISPR-Cas system may be harnessed to correct these defects of genomic instability. And thus, target sequences can be found in these defects of genomic instability.

Further embodiments of the invention relate to algorithms that lay the foundation of methods relating to CRISPR enzyme, e.g. Cas, specificity or off-target activity. In general, algorithms refer to an effective method expressed as a finite list of well defined instructions for calculating one or more functions of interest. Algorithms may be expressed in several kinds of notation, including but not limited to programming languages, flow charts, control tables, natural languages, mathematical formula and pseudocode. In a preferred embodiment, the algorithm may be expressed in a programming language that expresses the algorithm in a form that may be executed by a computer or a computer system.

Methods relating to CRISPR enzyme, e.g. Cas, specificity or off-target activity are based on algorithms that include but are not limited to the thermodynamic algorithm, multiplicative algorithm and positional algorithm. These algorithms take in an input of a sequence of interest and identify candidate target sequences to then provide an output of a ranking of candidate target sequences or a score associated with a particular target sequence based on predicted off-target sites. Candidate target sites may be selected by an end user or a customer based on considerations which include but are not limited to modification efficiency, number, or location of predicted off-target cleavage. In a more preferred embodiment, a candidate target site is unique or has minimal predicted off-target cleavage given the previous parameters. However, the functional relevance of potential off-target modification should also be considered when choosing a target site. In particular, an end user or a customer may consider whether the off-target sites occur within loci of known genetic function, i.e. protein-coding exons, enhancer regions, or intergenic regulatory elements. There may also be cell-type specific considerations, i.e. if an off-target site occurs in a locus that is not functionally relevant in the target cell type. Taken together, a end user or customer may then make an informed, application-specific selection of a candidate target site with minimal off-target modification.

The thermodynamic algorithm may be applied in selecting a CRISPR complex for targeting and/or cleavage of a candidate target nucleic acid sequence within a cell. The first step is to input the target sequence (Step S400) which may have been determined using the positional algorithm. A CRISPR complex is also input (Step S402). The next step is to compare the target sequence with the guide sequence for the CRISPR complex (Step S404) to identify any mismatches. Furthermore, the amount, location and nature of the mismatch(es) between the guide sequence of the potential CRISPR complex and the candidate target nucleic acid sequence may be determined. The hybridization free energy of binding between the target sequence and the guide sequence is then calculated (Step S406). For example, this may be calculated by determining a contribution of each of the amount, location and nature of mismatch(es) to the hybridization free energy of binding between the target nucleic acid sequence and the guide sequence of potential CRISPR complex(es). Furthermore, this may be calculated by applying a model calculated using a training data set as explained in more detail below. Based on the hybridization free energy (i.e. based on the contribution analysis) a prediction of the likelihood of cleavage at the location(s) of the mismatch(es) of the target nucleic acid sequence by the potential CRISPR complex(es) is generated (Step S408). The system then determines whether or not there are any additional CRISPR complexes to consider and if so repeats the comparing, calculating and predicting steps. Each CRISPR complex is selected from the potential CRISPR complex(es) based on whether the prediction indicates that it is more likely than not that cleavage will occur at location(s) of mismatch(es) by the CRISPR complex (Step S410). Optionally, the probabilities of cleavage may be ranked so that a unique CRISPR complex is selected. Determining the contribution of each of the amount, location and nature of mismatch(es) to hybridization free energy includes but is not limited to determining the relative contribution of these factors. The term “location” as used in the term “location of mismatch(es)” may refer to the actual location of the one or more base pair mismatch(es) but may also include the location of a stretch of base pairs that flank the base pair mismatch(es) or a range of locations/positions. The stretch of base pairs that flank the base pair mismatch(es) may include but are not limited to at least one, at least two, at least three base pairs, at least four or at least five or more base pairs on either side of the one or more mismatch(es). As used herein, the “hybridization free energy” may be an estimation of the free energy of binding, e.g. DNA:RNA free energy of binding which may be estimated from data on DNA:DNA free energy of binding and RNA:RNA free energy of binding.

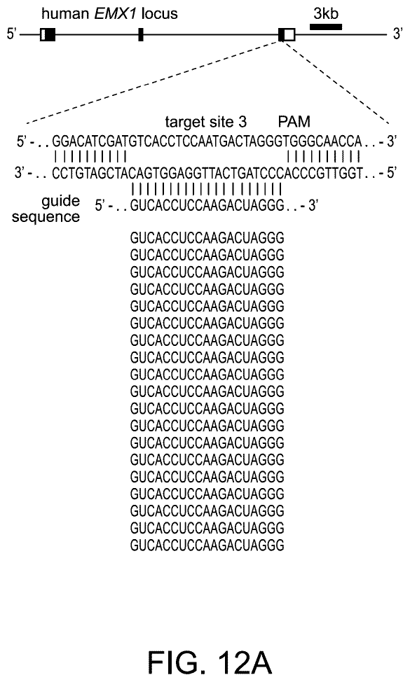

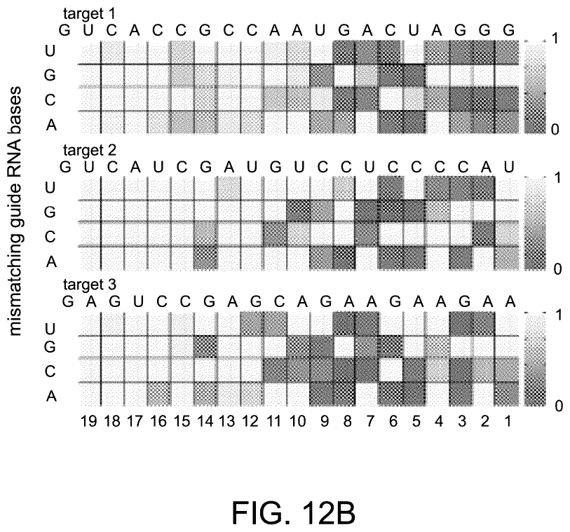

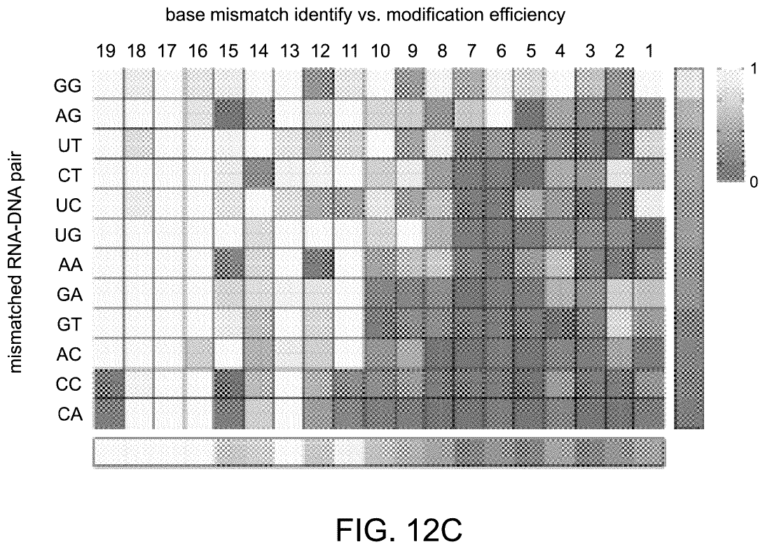

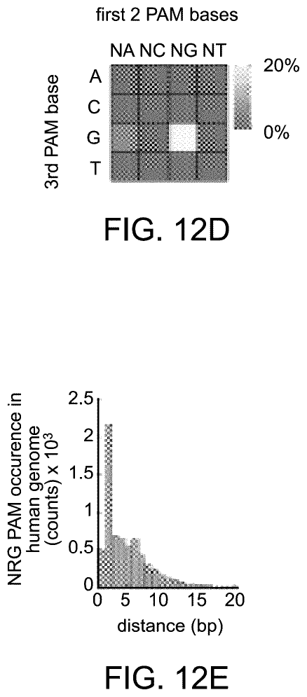

In methods relating to the multiplicative algorithm applied in identifying one or more unique target sequences in a genome of a eukaryotic organism, whereby the target sequence is susceptible to being recognized by a CRISPR-Cas system, wherein the method comprises: a) creating a data training set as to a particular Cas, b) determining average cutting frequency at a particular position for the particular Cas from the data training set, c) determining average cutting frequency of a particular mismatch for the particular Cas from the data training set, d) multiplying the average cutting frequency at a particular position by the average cutting frequency of a particular mismatch to obtain a first product, e) repeating steps b) to d) to obtain second and further products for any further particular position (s) of mismatches and particular mismatches and multiplying those second and further products by the first product, for an ultimate product, and omitting this step if there is no mismatch at any position or if there is only one particular mismatch at one particular position (or optionally e) repeating steps b) to d) to obtain second and further products for any further particular position (s) of mismatches and particular mismatches and multiplying those second and further products by the first product, for an ultimate product, and omitting this step if there is no mismatch at any position or if there is only one particular mismatch at one particular position), and f) multiplying the ultimate product by the result of dividing the minimum distance between consecutive mismatches by 18 and omitting this step if there is no mismatch at any position or if there is only one particular mismatch at one particular position (or optionally f) multiplying the ultimate product by the result of dividing the minimum distance between consecutive mismatches by 18 and omitting this step if there is no mismatch at any position or if there is only one particular mismatch at one particular position), to thereby obtain a ranking, which allows for the identification of one or more unique target sequences, the predicted cutting frequencies for genome-wide targets may be calculated by multiplying, in series: fₑₛₜ=f(1)g(N₁,N₁′)×f(2)g(N₂,N₂′)× . . . f(19)g(N₁₉,N₁₉′)×h with values f(i) and g(Nᵢ,Nᵢ¹) at position E corresponding, respectively, to the aggregate position- and base-mismatch cutting frequencies for positions and pairings indicated in a generalized base transition matrix or an aggregate matrix, e.g. a matrix as indicated in FIG. 12c. Each frequency was normalized to range from 0 to 1, such that f→(f−fₘᵢₙ)/(fₘₐₓ−fₘᵢₙ). In case of a match, both were set equal to 1. The value h meanwhile re-weighted the estimated frequency by the minimum pairwise distance between consecutive mismatches in the target sequence. This value distance, in base-pairs, was divided by 18 to give a maximum value of 1 (in cases where fewer than 2 mismatches existed, or where mismatches occurred on opposite ends of the 19 bp target-window). Samples having a read-count of at least 10,000 (n=43) were plotted. Those tied in rank were given a rank-average. The Spearman correlation coefficient, 0.58, indicated that the estimated frequencies recapitulated 58% of the rank-variance for the observed cutting frequencies. Comparing Jest with the cutting frequencies directly yielded a Pearson correlation of 0.89. While dominated by the highest-frequency gRNA/target pairs, this value indicated that nearly 90% of all cutting-frequency variance was explained by the predictions above. In further aspects of the invention, the multiplicative algorithm or the methods mentioned herein may also include thermodynamic factors, e.g. hybridization energies, or other factors of interest being multiplied in series to arrive at the ultimate product.

In embodiments of the invention, determining the off-target activity of a CRISPR enzyme may allow an end user or a customer to predict the best cutting sites in a genomic locus of interest. In a further embodiment of the invention, one may obtain a ranking of cutting frequencies at various putative off-target sites to verify in vitro, in vivo or ex vivo if one or more of the worst case scenario of non-specific cutting does or does not occur. In another embodiment of the invention, the determination of off-target activity may assist with selection of specific sites if an end user or customer is interested in maximizing the difference between on-target cutting frequency and the highest cutting frequency obtained in the ranking of off-target sites. Another aspect of selection includes reviewing the ranking of sites and identifying the genetic loci of the non-specific targets to ensure that a specific target site selected has the appropriate difference in cutting frequency from say targets that may encode for oncogenes or other genetic loci of interest. Aspects of the invention may include methods of minimizing therapeutic risk by verifying the off-target activity of the CRISPR-Cas complex. Further aspects of the invention may include utilizing information on off-target activity of the CRSIPR-Cas complex to create specific model systems (e.g. mouse) and cell lines. The methods of the invention allow for rapid analysis of non-specific effects and may increase the efficiency of a laboratory.

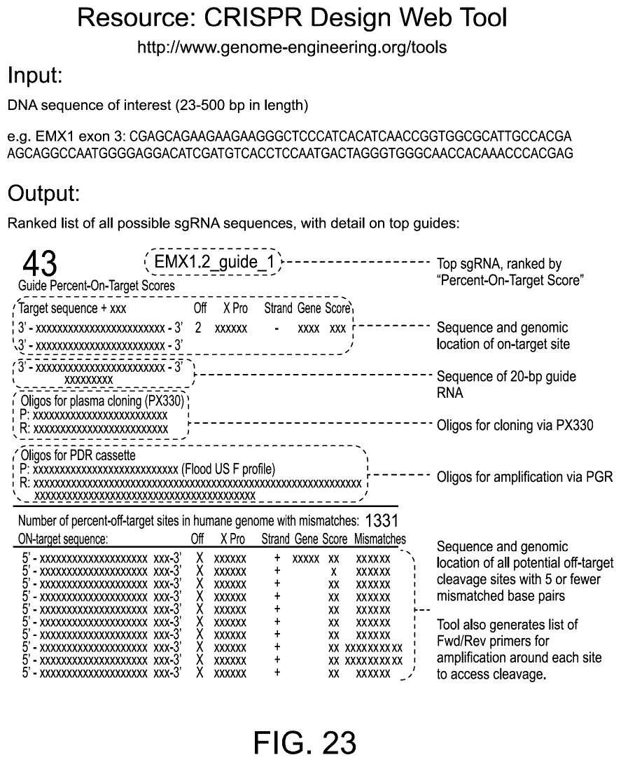

In methods relating to the positional algorithm applied in identifying one or more unique target sequences in a genome of a eukaryotic organism, whereby the target sequence is susceptible to being recognized by a CRISPR-Cas system, wherein the method comprises: a) determining average cutting frequency of guide-RNA/target mismatches at a particular position for a particular Cas from a training data set as to that Cas, if more than one mismatch, repeat step a) so as to determine cutting frequency for each mismatch, multiply frequencies of mismatches to thereby obtain a ranking, which allows for the identification of one or more unique target sequences, an example of an application of this algorithm may be seen in FIG. 23.

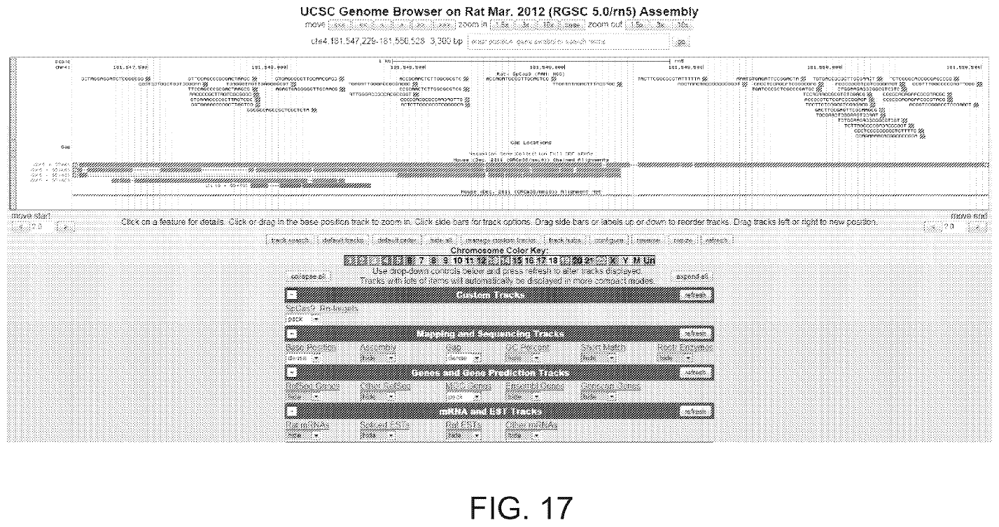

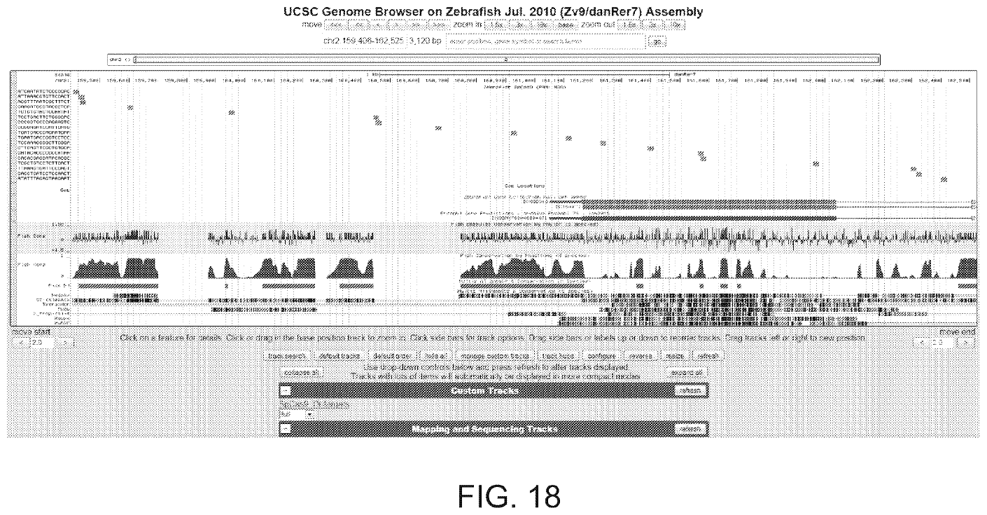

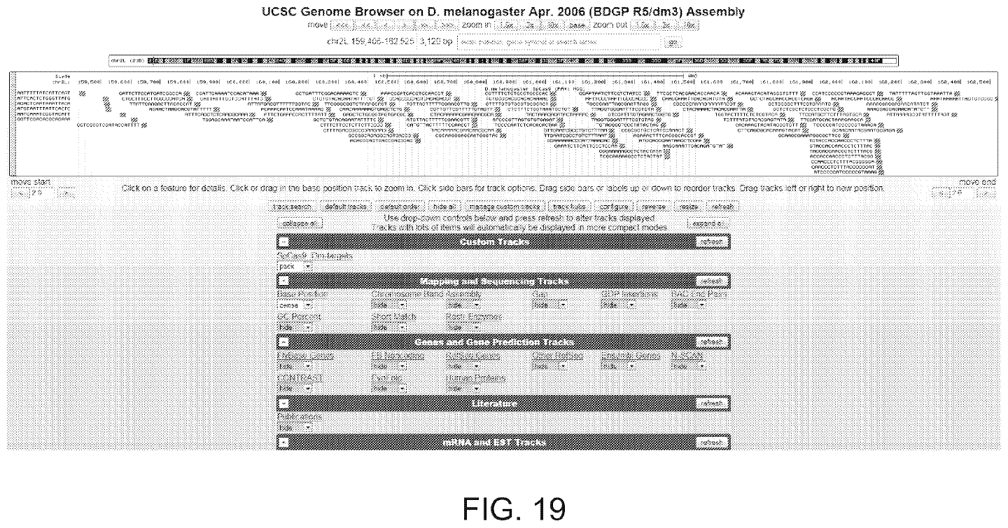

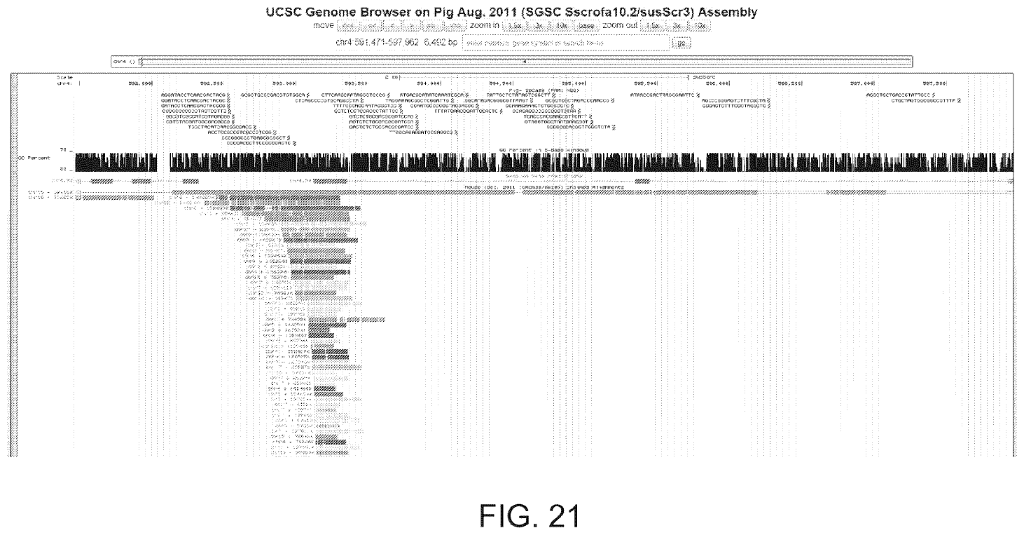

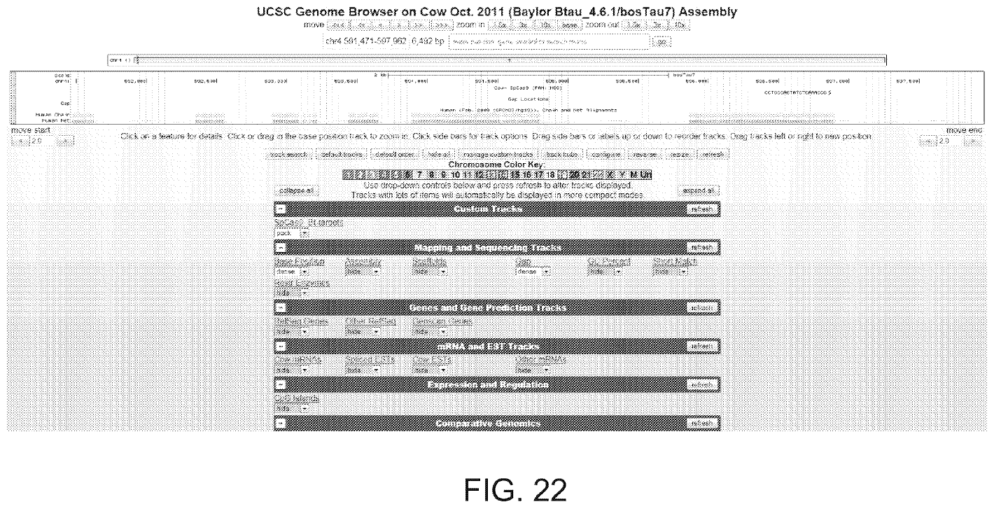

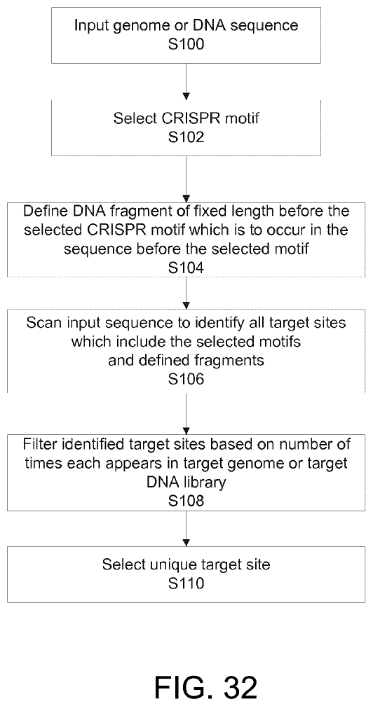

FIGS. 32, 33A, 33B and 34, respectively, each show a flow diagram of methods of the invention. FIG. 32 provides a flow diagram as to locational or positional methods of the invention, i.e., with respect to computational identification of unique CRISPR target sites: To identify unique target sites for a Cas, e.g., a Cas9, e.g., the S. pyogenes SF370 Cas9 (SpCas9) enzyme, in nucleic acid molecules, e.g., of cells, e.g., of organisms, which include but are not limited to human, mouse, rat, zebrafish, fruit fly, and C. elegans genome, Applicants developed a software package to scan both strands of a DNA sequence and identify all possible SpCas9 target sites. The method is shown in FIG. 32 which shows that the first step is to input the genome sequence (Step S100). The CRISPR motif(s) which are suitable for this genome sequence are then selected (Step S102). For this example, the CRISPR motif is an NGG protospacer adjacent motif (PAM) sequence. A fragment of fixed length which needs to occur in the overall sequence before the selected motif (i.e. upstream in the sequence) is then selected (Step S102). In this case, the fragment is a 20 bp sequence. Thus, each SpCas9 target site was is operationally defined as a 20 bp sequence followed by an NGG protospacer adjacent motif (PAM) sequence, and all sequences satisfying this 5′-N20-NGG-3′ definition on all chromosomes were identified (Step S106). To prevent non-specific genome editing, after identifying all potential sites, all target sites were filtered based on the number of times they appear in the relevant reference genome (Step S108). (Essentially, all the 20-bp fragments (candidate target sites) upstream of the NGG PAM motif are aggregated. If a particular 20-bp fragment occurs more than once in your genome-wide search, it is considered not unique and ‘strikes out’, aka filtered. The 20-bp fragments that REMAIN therefore occur only once in the target genome, making it unique; and, instead of taking a 20-bp fragment (the full Cas9 target site), this algorithm takes the first, for example, 11-12 bp upstream of the PAM motif and requires that to be unique.) Finally, a unique target site is selected (Step S110), e.g. To take advantage of sequence specificity of Cas, e.g., Cas9 activity conferred by a ‘seed’ sequence, which can be, for example, approximately 11-12 bp sequence 5′ from the PAM sequence, 5′-NNNNNNNNNN-NGG-3′ sequences were selected to be unique in the relevant genome. Genomic sequences are available on the UCSC Genome Browser and sample visualizations of the information for the Human genome hg, Mouse genome mm, Rat genome rn, Zebrafish genome danRer, D. melanogaster genome dm, C. elegans genome ce, the pig genome and cow genome are shown in FIGS. 15 through 22 respectively.

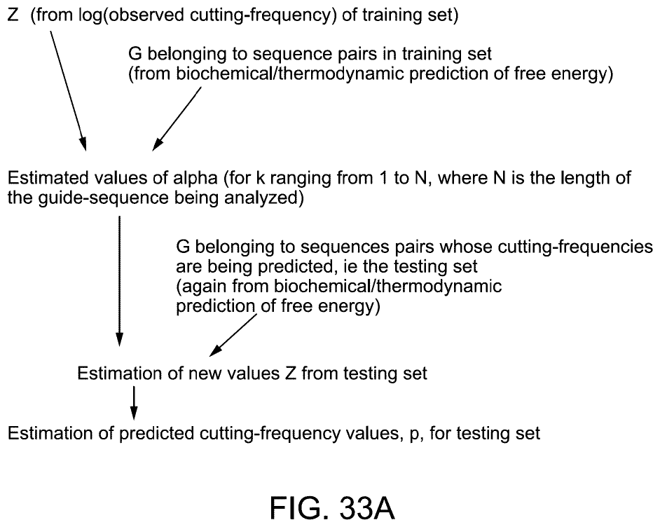

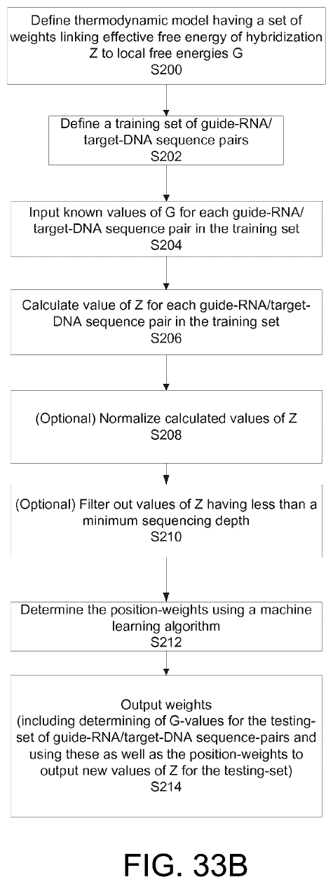

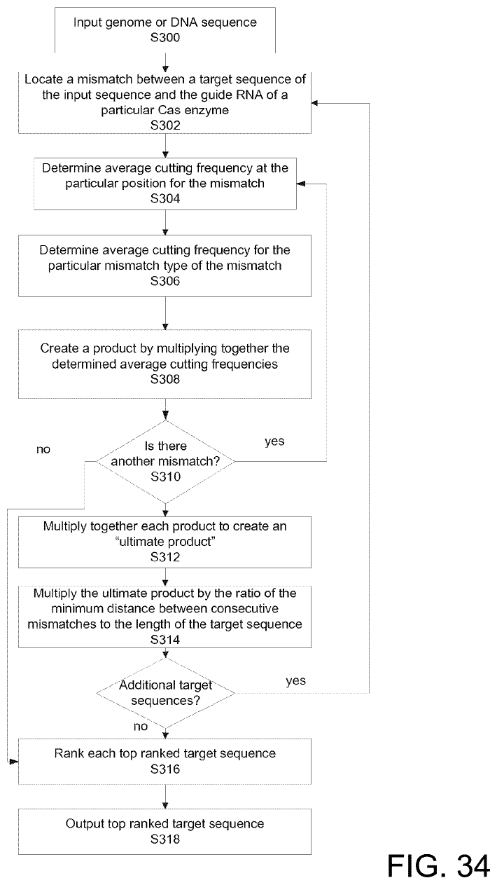

FIGS. 33A and 33B each provides a flow diagram as to thermodynamic methods of the invention. FIG. 34 provides a flow diagram as to multiplication methods of the invention. Referring to FIGS. 33A and 33B, and considering the least squares thermodynamic model of CRISPR-Cas cutting efficiency, for arbitrary Cas9 target sites, Applicants generated a numerical thermodynamic model that predicts Cas9 cutting efficiency. Applicants propose 1) that the Cas9 guide RNA has specific free energies of hybridization to its target and any off-target DNA sequences and 2) that Cas9 modifies RNA:DNA hybridization free-energies locally in a position-dependent but sequence-independent way. Applicants trained a model for predicting CRISPR-Cas cutting efficiency based on their CRISPR-Cas guide RNA mutation data and RNA:DNA thermodynamic free energy calculations using a machine learning algorithm. Applicants then validated their resulting models by comparing their predictions of CRISPR-Cas off-target cutting at multiple genomic loci with experimental data assessing locus modification at the same sites. The methodology adopted in developing this algorithm is as follows: The problem summary states that for arbitrary spacers and targets of constant length, a numerical model that makes thermodynamic sense and predicts Cas9 cutting efficiency is to be found. Suppose Cas9 modifies DNA:RNA hybridization free-energies locally in a position-dependent but sequence-independent way. The first step is to define a model having a set a weights which links the free energy of hybridization Z with the local free energies G (Step S200). Then for DNA:RNA hybridization free energies ΔGᵢⱼ(k) (for position k between 1 and N) of spacer i and target j

$${Z}_{ij} = {\sum }_{k = 1}^{N} {α}_{k} Δ {G}_{ij} ( k )$$

Zᵢⱼ can be treated as an “effective” free-energy modified by the multiplicative position-weights αₖ. The “effective” free-energy Zᵢⱼ corresponds to an associated cutting-probability ˜e^{-βZ}^{ᵢⱼ} (for some constant β) in the same way that an equilibrium model of hybridization (without position-weighting) would have predicted a hybridization-probability ˜e^{-βΔG}^{ᵢⱼ}) Since cutting-efficiency has been measured, the values Zᵢⱼ can be treated as their observables. Meanwhile, ΔGᵢⱼ(k) can be calculated for any experiment's spacer-target pairing. Applicants task was to find the values αₖ, since this would allow them to estimate Zᵢⱼ for any spacer-target pair. The weights are determined by inputting known values for Z and G from a training set of sequences with the known values being determined by experimentation as necessary. Thus, Applicants need to define a training set of sequences (Step S202) and calculate a value of Z for each sequence in the training set (Step S204). Writing the above equation for Zᵢⱼ in matrix form Applicants get:

$$\vec{Z} = G \vec{α} \qquad ( 1 )$$

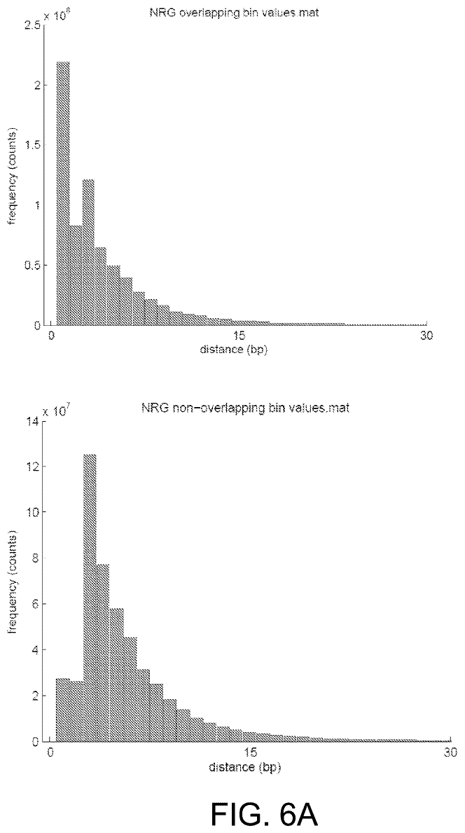

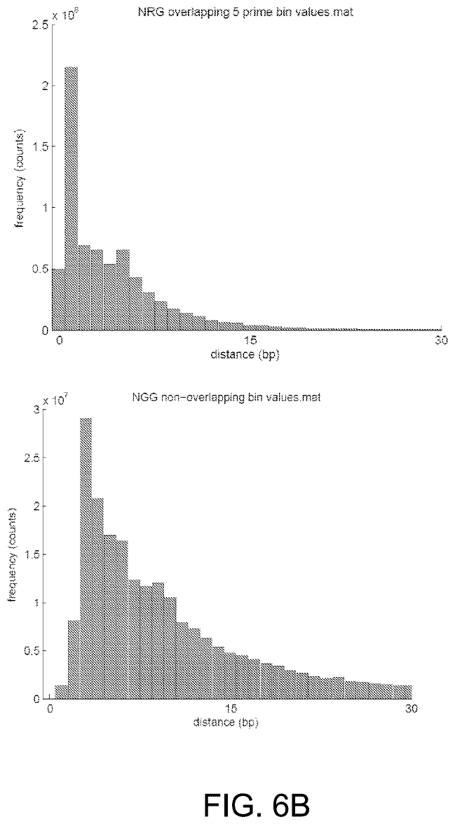

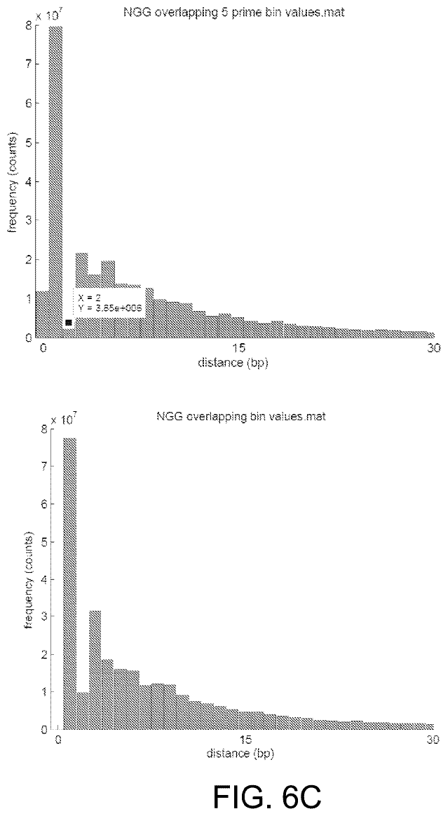

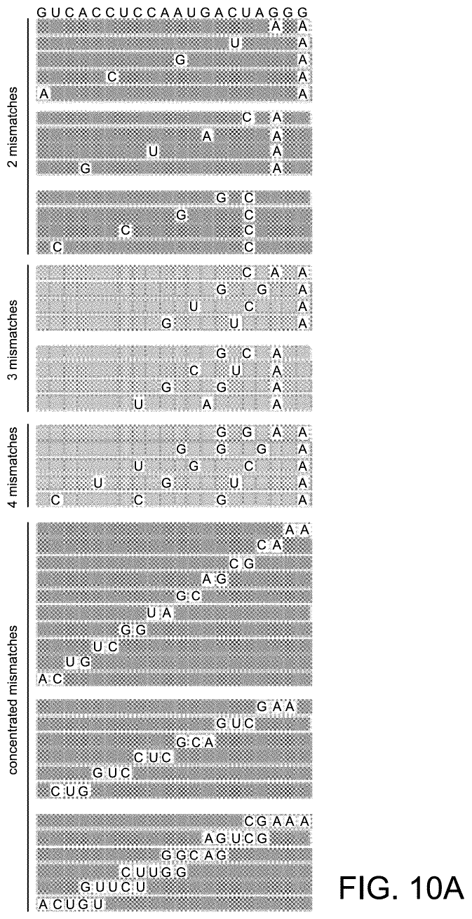

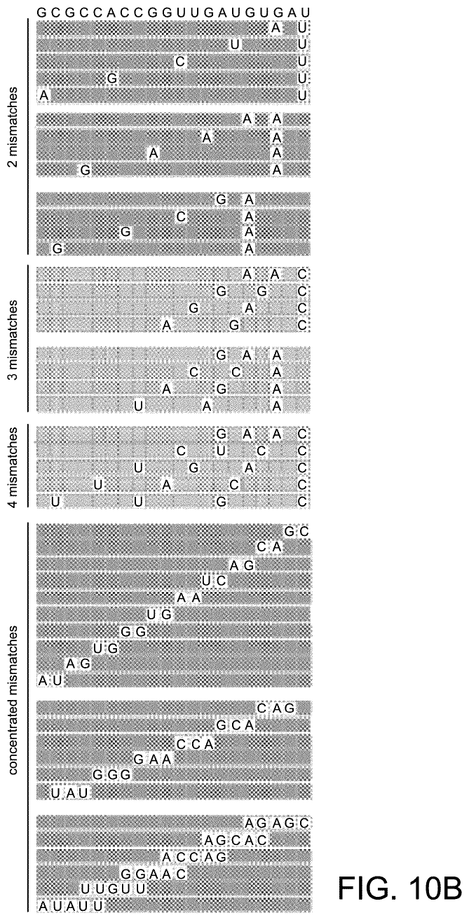

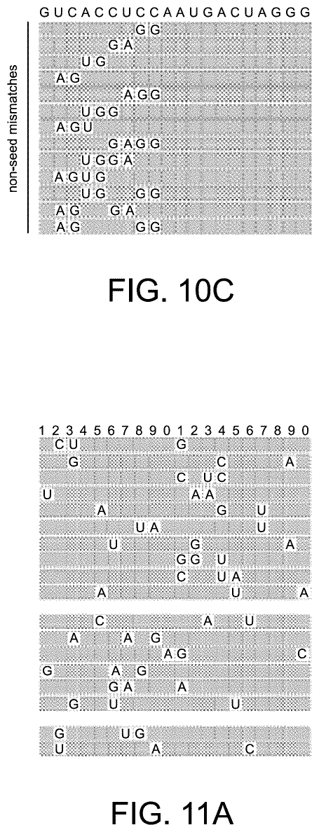

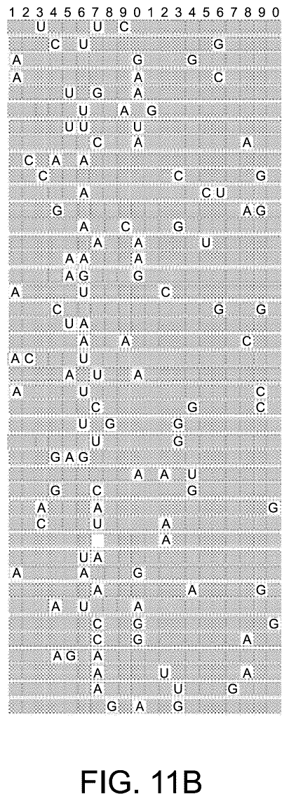

The least-squares estimate is then

$${\vec{a}}_{est} = {( {G}^{T} G )}^{- 1} {G}^{T} \overline{Z}$$

where G^{T} is the matrix-transpose of the G and (G^{T}G)⁻¹ is the inverse of their matrix-product. In the above G is a matrix of local DNA:RNA free-energy values whose rth row corresponds to experimental trial r and whose kth column corresponds to the kth position in the DNA:RNA hybrid tested in that experimental trial. These values of G are thus input into the training system (Step S204). \vec{Z} is meanwhile a column-vector whose rth row corresponds to observables from the same experimental trial as G's rth row. Because of the relation described above wherein the CRISPR cutting frequencies are estimated to vary as ˜e^{-βZ}^{ᵢⱼ}, these observables, Zᵢⱼ, were calculated as the natural logarithm of the observed cutting frequency. The observable is the cleavage efficiency of Cas, e.g., Cas9, at a target DNA for a particular guide RNA and target DNA pair. The experiment is Cas, e.g., Cas9, with a particular sgRNA/DNA target pairing, and the observable is the cleavage percentage (whether measured as indel formation percentage from cells or simply cleavage percentage in vitro) (see herein discussion on generating training data set). More in particular, every unique PCR reaction that was sequenced should be treated as a unique experimental trial to encompass replicability within the vector. This means that experimental replicates each go into separate rows of equation 1 (and because of this, some rows of G will be identical). The advantage of this is that when \vec{a} is fit, all relevant information—including replicability—is taken into account in the final estimate. Observable \vec{Z}, values were calculated as log (observed frequency of cutting) (Step S206). Cutting frequencies were optionally normalized identically (so that they all have the same “units”) (Step S208). For plugging in sequencing indel-frequency values, it may be best, however, to standardize sequencing depth. The preferred way to do this would be to set a standard sequencing-depth D for which all experiments included in \vec{Z} have at least that number of reads. Since cutting frequencies below 1/D cannot be consistently detected, this should be set as the minimum frequency for the data-set, and the values in \vec{Z} should range from log(1/D) to log(1). One could vary the value of D later on to ensure that the \vec{a} estimate isn't too dependent on the value chosen. Thus, values of Z could be filtered out if they do not meet the minimum sequencing depth (Step S210). Once the values of G and Z are input to the machine learning system, the weights can be determined (Step S212) and output (Step S214). These weights can then be used to estimate the free energy Z and the cutting frequency for any sequence. In a further aspect, there are different methods of graphing NGG and NNAGAAW sequences. One is with the ‘non-overlapping’ method. NGG and NRG may be regraphed in an “overlapping” fashion, as indicated in FIGS. 6 A-C. Applicants also performed a study on off target Cas9 activity as indicated in FIGS. 10, 11 and 12. Aspects of the invention also relate to predictive models that may not involve hybridization energies but instead simply use the cutting frequency information as a prediction.

FIG. 34 shows the steps in one method relating to the multiplicative algorithm which may be applied in identifying one or more unique target sequences in a genome of a eukaryotic organism, whereby the target sequence is susceptible to being recognized by a CRISPR-Cas system. The method comprises: a) creating a data training set as to a particular Cas. The data training set may be created as described in more detail later by determining the weights associated with a model. Once a data training set has been established, it can be used to predict the behavior of an input sequence and to identify one or more unique target sequences therein. At step S300, the genome sequence is input to the system. For a particular Cas, the next step is to locate a mismatch between a target sequence within the input sequence and guide RNA for the particular Cas (Step S302). For the identified mismatch, two average cutting frequencies are determined using the data training set. These are the average cutting frequency at the position of the mismatch (step S304) and the average cutting frequency associated with that type of mismatch (Step S306). These average cutting frequencies are determined from the data training set which is particular to that Cas. The next step S308 is to create a product by multiplying the average cutting frequency at a particular position by the average cutting frequency of a particular mismatch to obtain a first product. It is then determined at step S310 whether or not there are any other mismatches. If there are none, the target sequence is output as the unique target sequence. However, if there are other mismatches, steps 304 to 308 are repeated to obtain second and further products for any further particular position (s) of mismatches and particular mismatches. Where second and further products are created and all products are multiplied together to create an ultimate product. The ultimate product is then multiplied by the result of dividing the minimum distance between consecutive mismatches by the length of the target sequence (e.g. 18) (step S314) which effectively scales each ultimate product. It will be appreciated that steps 312 and 314 are omitted if there is no mismatch at any position or if there is only one particular mismatch at one particular position. The process is then repeated for any other target sequences. The “scaled” ultimate products for each target sequence are each ranked to thereby obtain a ranking (Step S316), which allows for the identification of one or more unique target sequences by selecting the highest ranked one (Step S318). Thus the “scaled” ultimate product which represents the predicted cutting frequencies for genome-wide targets may be calculated by: fₑₛₜ=f(1)g(N₁,N₁′)×f(2)g(N₂,N₂′)× . . . f(19)g(N₁₉,N₁₉′)×h with values f(i) and g(Nᵢ,Nᵢ′) at position i corresponding, respectively, to the aggregate position- and base-mismatch cutting frequencies for positions and pairings indicated in a generalized base transition matrix or an aggregate matrix, e.g. a matrix as indicated in FIG. 12c. In other words, f(i) is the average cutting frequency at the particular position for the mismatch and g(Nᵢ, N′ᵢ) is the average cutting frequency for the particular mismatch type for the mismatch. Each frequency was normalized to range from 0 to 1, such that f→(f−fₘᵢₙ)/(fₘₐₓ−fₘᵢₙ). In case of a match, both were set equal to 1. The value h meanwhile re-weighted the estimated frequency by the minimum pairwise distance between consecutive mismatches in the target sequence. This value distance, in base-pairs, was divided by a constant which was indicative of the length of the target sequence (e.g. 18) to give a maximum value of 1 (in cases where fewer than 2 mismatches existed, or where mismatches occurred on opposite ends of the 19 bp target-window). Samples having a read-count of at least 10,000 (n=43) were plotted. Those tied in rank were given a rank-average. The Spearman correlation coefficient, 0.58, indicated that the estimated frequencies recapitulated 58% of the rank-variance for the observed cutting frequencies. Comparing Jest with the cutting frequencies directly yielded a Pearson correlation of 0.89. While dominated by the highest-frequency gRNA/target pairs, this value indicated that nearly 90% of all cutting-frequency variance was explained by the predictions above. In further aspects of the invention, the multiplicative algorithm or the methods mentioned herein may also include thermodynamic factors, e.g. hybridization energies, or other factors of interest being multiplied in series to arrive at the ultimate product.

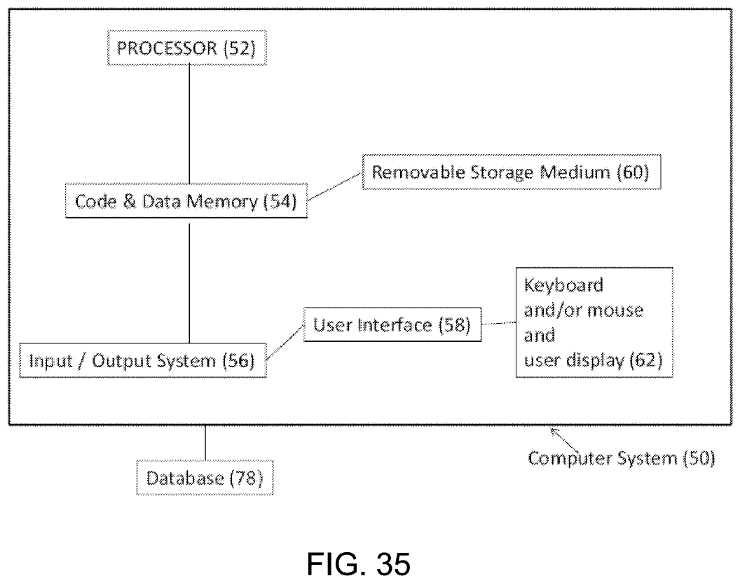

FIG. 35 shows a schematic block diagram of a computer system which can be used to implement the methods described herein. The computer system 50 comprises a processor 52 coupled to code and data memory 54 and an input/output system 56 (for example comprising interfaces for a network and/or storage media and/or other communications). The code and/or data stored in memory 54 may be provided on a removable storage medium 60. There may also be a user interface 58 for example comprising a keyboard and/or mouse and a user display 62. The computer system is connected to a database 78. The database 78 comprises the data associated with the data training sets. The computer system is shown as a single computing device with multiple internal components which may be implemented from a single or multiple central processing units, e.g. microprocessors. It will be appreciated that the functionality of the device may be distributed across several computing devices. It will also be appreciated that the individual components may be combined into one or more components providing the combined functionality. Moreover, any of the modules, databases or devices shown may be implemented in a general purpose computer modified (e.g. programmed or configured) by software to be a special-purpose computer to perform the functions described herein. The processor may be configured to carry out the steps shown in the various flowcharts. The user interface may be used to input the genome sequence, the CRISPR motif and/or Cas for which a target sequence is to be identified. The output unique target sequence(s) may be displayed on the user display.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Evaluation of the Specificity of Cas9-Mediated Genome Cleavage

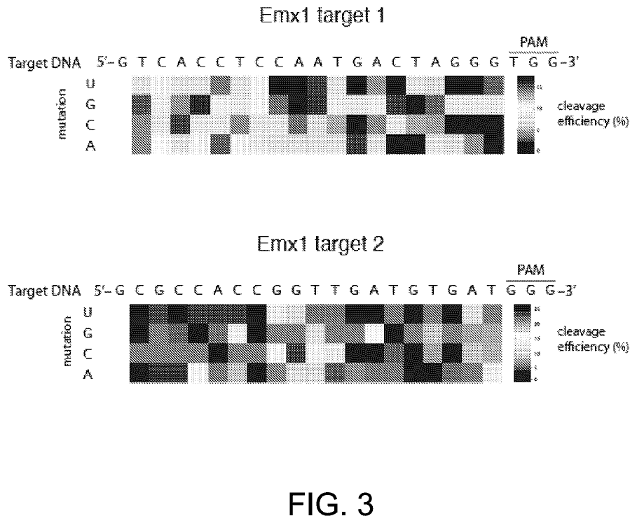

Applicants carried out an initial test to evaluate the cleavage specificity of Cas9 from Streptococcus pyogenes. The assay was designed to test the effect of single basepair mismatches between the guide RNA sequence and the target DNA. The results from the initial round of testing are depicted in FIG. 3.

Applicants carried out the assay using 293FT cells in 96 well plates. Cells were transfected with 65 ng of a plasmid carrying Cas9 and 10 ng of a PCR amplicon carrying the pol3 promoter U6 and the guide RNA. The experiment was conducted using a high amount of Cas9 and guide RNA, which probably explains the seemingly low specificity (i.e. single base mismatches is not sufficient to abolish cleavage). Applicants also evaluate the effect of different concentration of Cas9 and RNA on cleavage specificity. Additionally, Applicants carry out a comprehensive evaluation of every possible mismatch in each position of the guide RNA. The end goal is to generate a model to inform the design of guide RNAs having high cleavage specificity.

Additional experiments test position and number of mismatches in the guide RNA on cleavage efficiency. The following table shows a list of 48 mismatch possibilities. In the table 0 means no mutation and 1 means with mutation.

| | | | | | | | | | | | | | | | | | | | | |
|---|---|---|---|---|---|---|---|---|---|---|---|---|---|---|---|---|---|---|---|---|
| 20 | 19 | 18 | 17 | 16 | 15 | 14 | 13 | 12 | 11 | 10 | 9 | 8 | 7 | 6 | 5 | 4 | 3 | 2 | 1 | NGG |

Test Rule 1: More mismatches = bigger effect on cutting
Test Rule 2: Mismatches on 5′ end have less effect than mismatches on 3′ end

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 1 | 1 |
| 2 | 0 | 0 | 0 | 0 | 0 | 0 | 0 | 0 | 0 | 0 | 0 | 0 | 0 | 0 | 0 | 0 | 1 | 1 | 0 | 0 |
| 3 | 0 | 0 | 0 | 0 | 0 | 0 | 0 | 0 | 0 | 0 | 0 | 0 | 0 | 0 | 1 | 1 | 0 | 0 | 0 | 0 |
| 4 | 0 | 0 | 0 | 0 | 0 | 0 | 0 | 0 | 0 | 0 | 0 | 0 | 1 | 1 | 0 | 0 | 0 | 0 | 0 | 0 |
| 5 | 0 | 0 | 0 | 0 | 0 | 0 | 0 | 0 | 0 | 0 | 1 | 1 | 0 | 0 | 0 | 0 | 0 | 0 | 0 | 0 |
| 6 | 0 | 0 | 0 | 0 | 0 | 0 | 0 | 0 | 1 | 1 | 0 | 0 | 0 | 0 | 0 | 0 | 0 | 0 | 0 | 0 |
| 7 | 0 | 0 | 0 | 0 | 0 | 0 | 1 | 1 | 0 | 0 | 0 | 0 | 0 | 0 | 0 | 0 | 0 | 0 | 0 | 0 |
| 8 | 0 | 0 | 0 | 0 | 1 | 1 | 0 | 0 | 0 | 0 | 0 | 0 | 0 | 0 | 0 | 0 | 0 | 0 | 0 | 0 |
| 9 | 0 | 0 | 1 | 1 | 0 | 0 | 0 | 0 | 0 | 0 | 0 | 0 | 0 | 0 | 0 | 0 | 0 | 0 | 0 | 0 |
| 10 | 1 | 1 | 0 | 0 | 0 | 0 | 0 | 0 | 0 | 0 | 0 | 0 | 0 | 0 | 0 | 0 | 0 | 0 | 0 | 0 |
| 11 | 0 | 0 | 0 | 0 | 0 | 0 | 0 | 0 | 0 | 0 | 0 | 0 | 0 | 0 | 0 | 0 | 1 | 1 | 1 | 0 |
| 12 | 0 | 0 | 0 | 0 | 0 | 0 | 0 | 0 | 0 | 0 | 0 | 0 | 0 | 0 | 0 | 1 | 1 | 1 | 1 | 1 |
| 13 | 0 | 0 | 0 | 0 | 0 | 0 | 0 | 0 | 0 | 0 | 0 | 0 | 0 | 1 | 1 | 1 | 0 | 0 | 0 | 0 |
| 14 | 0 | 0 | 0 | 0 | 0 | 0 | 0 | 0 | 0 | 0 | 0 | 0 | 1 | 1 | 1 | 1 | 1 | 0 | 0 | 0 |
| 15 | 0 | 0 | 0 | 0 | 0 | 0 | 0 | 0 | 0 | 0 | 1 | 1 | 1 | 0 | 0 | 0 | 0 | 0 | 0 | 0 |
| 16 | 0 | 0 | 0 | 0 | 0 | 0 | 0 | 0 | 0 | 1 | 1 | 1 | 1 | 1 | 0 | 0 | 0 | 0 | 0 | 0 |
| 17 | 0 | 0 | 0 | 0 | 0 | 0 | 0 | 1 | 1 | 1 | 0 | 0 | 0 | 0 | 0 | 0 | 0 | 0 | 0 | 0 |
| 18 | 0 | 0 | 0 | 0 | 0 | 0 | 1 | 1 | 1 | 1 | 1 | 0 | 0 | 0 | 0 | 0 | 0 | 0 | 0 | 0 |
| 19 | 0 | 0 | 0 | 0 | 1 | 1 | 1 | 0 | 0 | 0 | 0 | 0 | 0 | 0 | 0 | 0 | 0 | 0 | 0 | 0 |
| 20 | 0 | 0 | 0 | 1 | 1 | 1 | 1 | 1 | 0 | 0 | 0 | 0 | 0 | 0 | 0 | 0 | 0 | 0 | 0 | 0 |
| 21 | 0 | 1 | 1 | 1 | 0 | 0 | 0 | 0 | 0 | 0 | 0 | 0 | 0 | 0 | 0 | 0 | 0 | 0 | 0 | 0 |
| 22 | 1 | 1 | 1 | 1 | 1 | 0 | 0 | 0 | 0 | 0 | 0 | 0 | 0 | 0 | 0 | 0 | 0 | 0 | 0 | 0 |

Test Rule 3: Mismatches more spreadout have less effect than mismatches more concentrated

| | | | | | | | | | | | | | | | | | | | | |
|---|---|---|---|---|---|---|---|---|---|---|---|---|---|---|---|---|---|---|---|---|
| 23 | 0 | 0 | 0 | 0 | 0 | 0 | 0 | 0 | 0 | 0 | 0 | 0 | 0 | 0 | 0 | 0 | 0 | 1 | 0 | 1 |
| 24 | 0 | 0 | 0 | 0 | 0 | 0 | 0 | 0 | 0 | 0 | 0 | 0 | 0 | 0 | 1 | 0 | 0 | 0 | 0 | 1 |
| 25 | 0 | 0 | 0 | 0 | 0 | 0 | 0 | 0 | 0 | 0 | 1 | 0 | 0 | 0 | 0 | 0 | 0 | 0 | 0 | 1 |
| 26 | 0 | 0 | 0 | 0 | 0 | 1 | 0 | 0 | 0 | 0 | 0 | 0 | 0 | 0 | 0 | 0 | 0 | 0 | 0 | 1 |
| 27 | 1 | 0 | 0 | 0 | 0 | 0 | 0 | 0 | 0 | 0 | 0 | 0 | 0 | 0 | 0 | 0 | 0 | 0 | 0 | 1 |
| 28 | 0 | 0 | 0 | 0 | 0 | 0 | 0 | 0 | 0 | 0 | 0 | 0 | 0 | 0 | 0 | 1 | 0 | 1 | 0 | 0 |
| 29 | 0 | 0 | 0 | 0 | 0 | 0 | 0 | 0 | 0 | 0 | 0 | 0 | 1 | 0 | 0 | 0 | 0 | 1 | 0 | 0 |
| 30 | 0 | 0 | 0 | 0 | 0 | 0 | 0 | 0 | 1 | 0 | 0 | 0 | 0 | 0 | 0 | 0 | 0 | 1 | 0 | 0 |
| 31 | 0 | 0 | 0 | 1 | 0 | 0 | 0 | 0 | 0 | 0 | 0 | 0 | 0 | 0 | 0 | 0 | 0 | 1 | 0 | 0 |
| 32 | 0 | 0 | 0 | 0 | 0 | 0 | 0 | 0 | 0 | 0 | 0 | 0 | 0 | 1 | 0 | 1 | 0 | 0 | 0 | 0 |
| 33 | 0 | 0 | 0 | 0 | 0 | 0 | 0 | 0 | 0 | 0 | 1 | 0 | 0 | 0 | 0 | 1 | 0 | 0 | 0 | 0 |
| 34 | 0 | 0 | 0 | 0 | 0 | 0 | 1 | 0 | 0 | 0 | 0 | 0 | 0 | 0 | 0 | 1 | 0 | 0 | 0 | 0 |
| 35 | 0 | 1 | 0 | 0 | 0 | 0 | 0 | 0 | 0 | 0 | 0 | 0 | 0 | 0 | 0 | 1 | 0 | 0 | 0 | 0 |
| 36 | 0 | 0 | 0 | 0 | 0 | 0 | 0 | 0 | 0 | 0 | 0 | 0 | 0 | 0 | 0 | 1 | 0 | 1 | 0 | 1 |
| 37 | 0 | 0 | 0 | 0 | 0 | 0 | 0 | 0 | 0 | 0 | 0 | 0 | 0 | 1 | 0 | 0 | 1 | 0 | 0 | 1 |
| 38 | 0 | 0 | 0 | 0 | 0 | 0 | 0 | 0 | 0 | 0 | 0 | 1 | 0 | 0 | 0 | 1 | 0 | 0 | 0 | 1 |
| 39 | 0 | 0 | 0 | 0 | 0 | 0 | 0 | 0 | 0 | 1 | 0 | 0 | 0 | 0 | 1 | 0 | 0 | 0 | 0 | 1 |
| 40 | 0 | 0 | 0 | 0 | 0 | 0 | 0 | 0 | 0 | 0 | 0 | 0 | 0 | 1 | 0 | 1 | 0 | 1 | 0 | 0 |
| 41 | 0 | 0 | 0 | 0 | 0 | 0 | 0 | 0 | 0 | 0 | 0 | 1 | 0 | 0 | 1 | 0 | 0 | 1 | 0 | 0 |
| 42 | 0 | 0 | 0 | 0 | 0 | 0 | 0 | 0 | 0 | 1 | 0 | 0 | 0 | 1 | 0 | 0 | 0 | 1 | 0 | 0 |
| 43 | 0 | 0 | 0 | 0 | 0 | 0 | 0 | 1 | 0 | 0 | 0 | 0 | 1 | 0 | 0 | 0 | 0 | 1 | 0 | 0 |
| 44 | 0 | 0 | 0 | 0 | 0 | 0 | 0 | 0 | 0 | 0 | 0 | 0 | 0 | 1 | 0 | 1 | 0 | 1 | 0 | 1 |
| 45 | 0 | 0 | 0 | 0 | 0 | 0 | 0 | 0 | 0 | 0 | 1 | 0 | 0 | 1 | 0 | 0 | 1 | 0 | 0 | 1 |
| 46 | 0 | 0 | 0 | 0 | 0 | 0 | 0 | 1 | 0 | 0 | 0 | 1 | 0 | 0 | 0 | 1 | 0 | 0 | 0 | 1 |
| 47 | 0 | 0 | 0 | 0 | 1 | 0 | 0 | 0 | 0 | 1 | 0 | 0 | 0 | 0 | 1 | 0 | 0 | 0 | 0 | 1 |
| 48 | 0 | 1 | 0 | 0 | 0 | 0 | 0 | 1 | 0 | 0 | 0 | 0 | 0 | 1 | 0 | 0 | 0 | 0 | 0 | 1 |

### Example 2: Evaluation of Mutations in the PAM Sequence, and its Effect on Cleavage Efficiency

Applicants tested mutations in the PAM sequence and its effect on cleavage. The PAM sequence for Streptococcus pyogenes Cas9 is NGG, where the GG is thought to be required for cleavage. To test whether Cas9 can cleavage sequences with PAMs that are different than NGG, Applicants chose the following 30 target sites from the Emx1 locus of the human genome—2 for each of the 15 PAM possibilities: NAA, NAC, NAT, NAG, NCA, NCC, NCG, NCT, NTA, NTC, NTG, NTT, NGA, NGC, and NGT; NGG is not selected because it can be targeted efficiently.

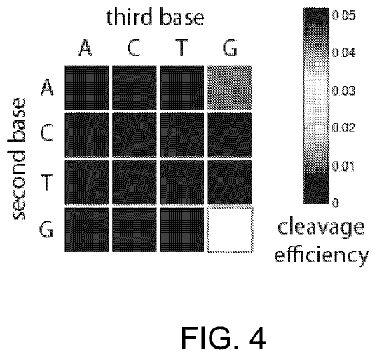

The cleavage efficiency data is shown in FIG. 4. The data shows that other than NGG, only sequences with NAG PAMs can be targeted.

| | Target 1 | Target 2 |
|---|---|---|
| | (SEQ ID NOS 28-42, | (SEQ ID NOS 43-57, |
| | respectively, | respectively, |
| | in order of | in order of |
| PAM | appearance) | appearance) |
| NAA | AGGCCCCAGTGGCTGCTCT | TCATCTGTGCCCCTCCCTC |
| NAT | ACATCAACCGGTGGCGCAT | GGGAGGACATCGATGTCAC |
| NAC | AAGGTGTGGTTCCAGAACC | CAAACGGCAGAAGCTGGAG |
| NAG | CCATCACATCAACCGGTGG | GGGTGGGCAACCACAAACC |
| NTA | AAACGGCAGAAGCTGGAGG | GGTGGGCAACCACAAACCC |
| NTT | GGCAGAAGCTGGAGGAGGA | GGCTCCCATCACATCAACC |
| NTC | GGTGTGGTTCCAGAACCGG | GAAGGGCCTGAGTCCGAGC |
| NTG | AACCGGAGGACAAAGTACA | CAACCGGTGGCGCATTGCC |
| NCA | TTCCAGAACCGGAGGACAA | AGGAGGAAGGGCCTGAGTC |
| NCT | GTGTGGTTCCAGAACCGGA | AGCTGGAGGAGGAAGGGCC |
| NCC | TCCAGAACCGGAGGACAAA | GCATTGCCACGAAGCAGGC |
| NCG | CAGAAGCTGGAGGAGGAAG | ATTGCCACGAAGCAGGCCA |
| NGA | CATCAACCGGTGGCGCATT | AGAACCGGAGGACAAAGTA |
| NGT | GCAGAAGCTGGAGGAGGAA | TCAACCGGTGGCGCATTGC |
| NGC | CCTCCCTCCCTGGCCCAGG | GAAGCTGGAGGAGGAAGGG |

### Example 3: Cas9 Diversity and RNAs, PAMS, Targets

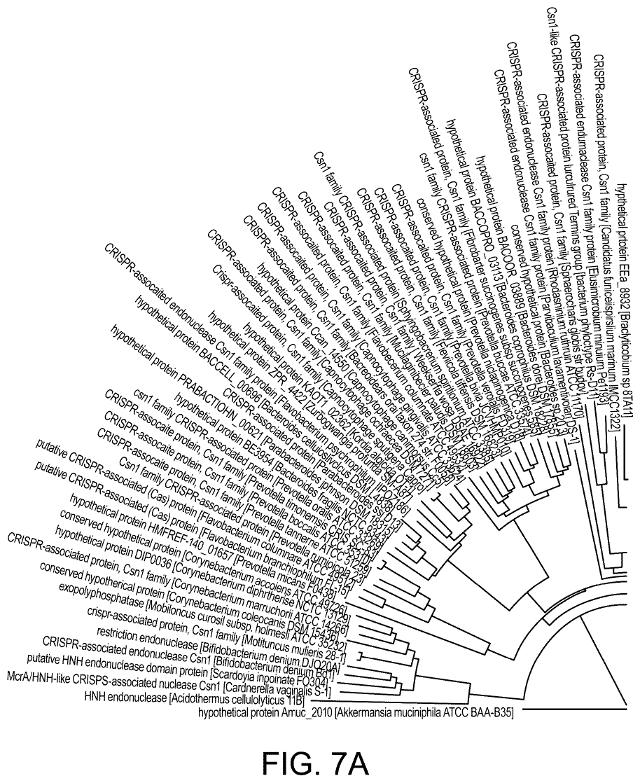

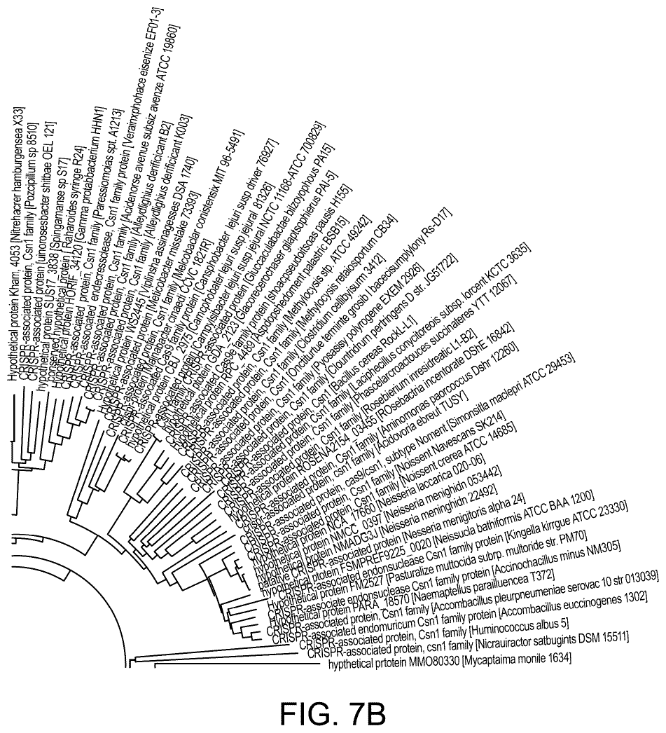

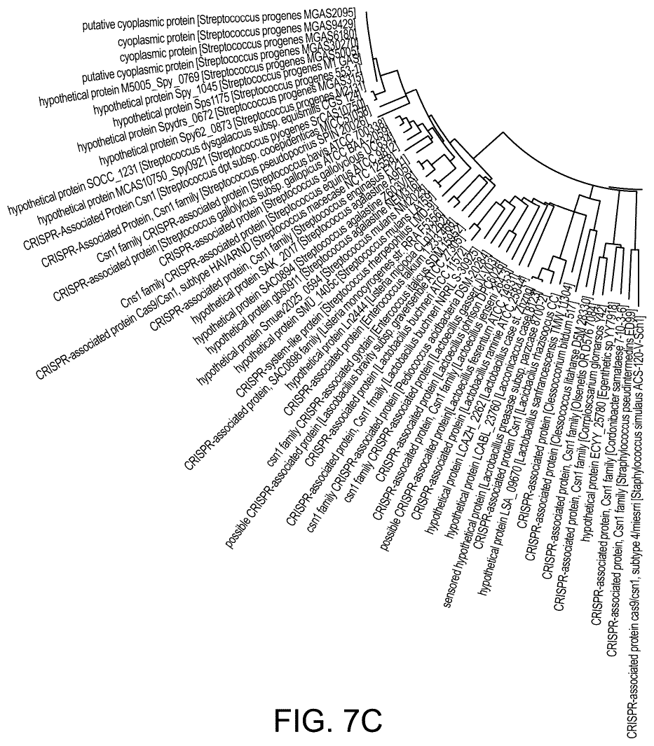

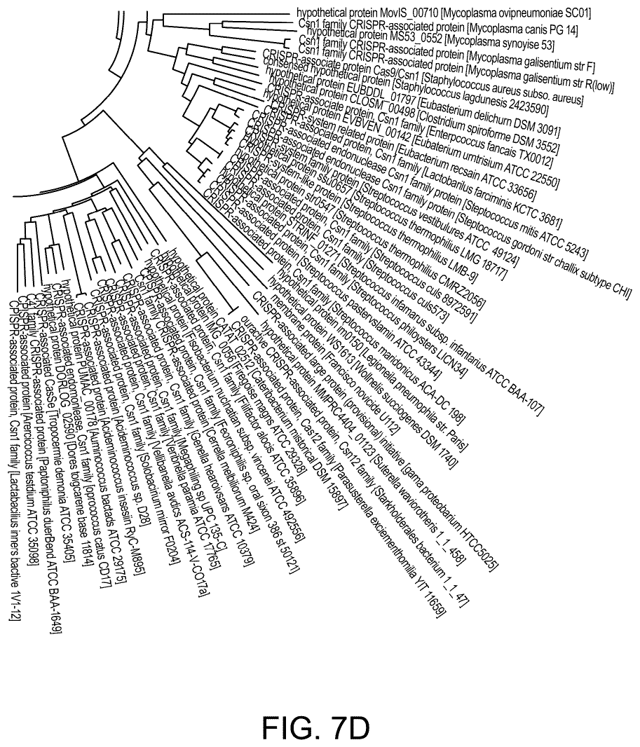

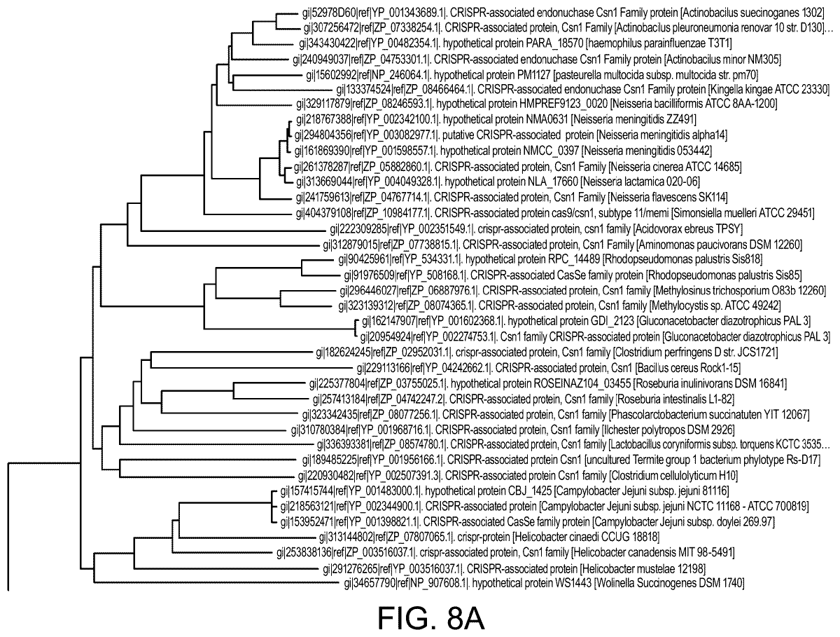

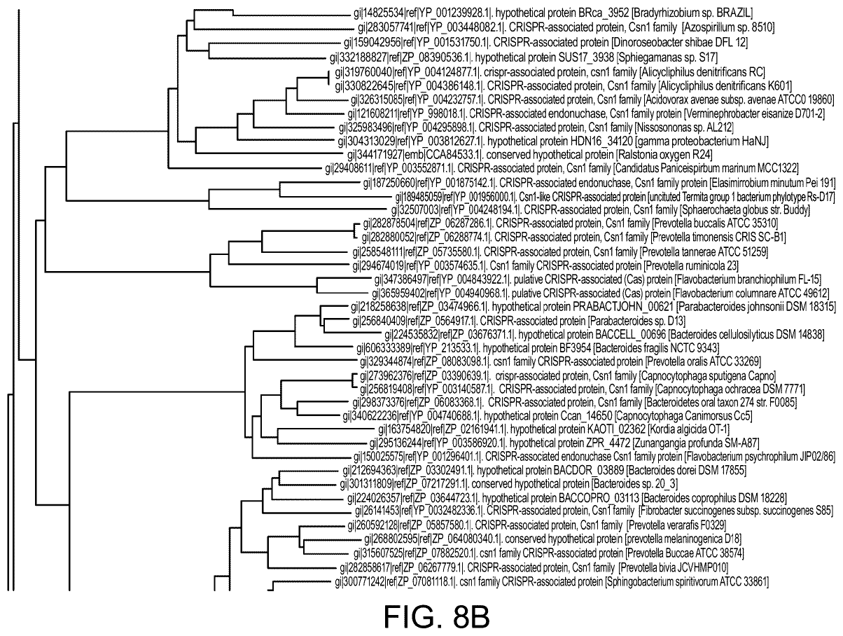

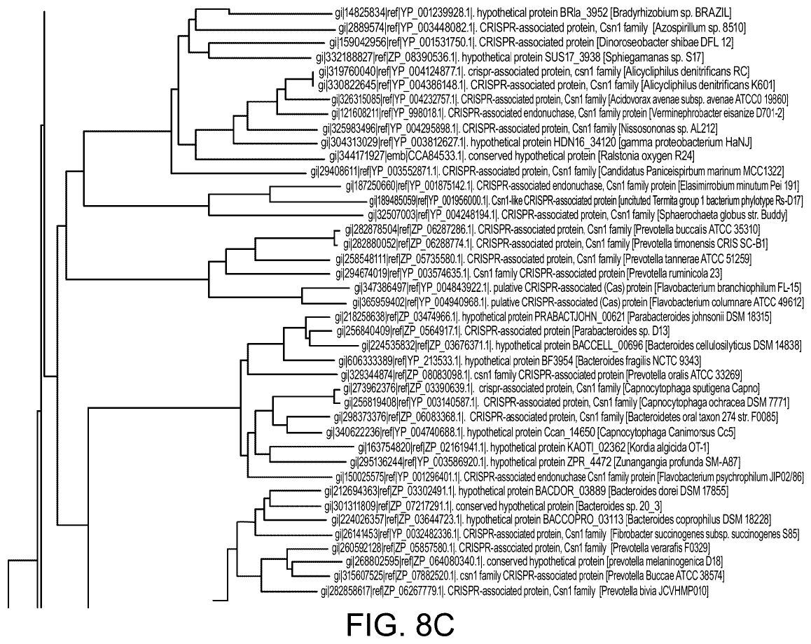

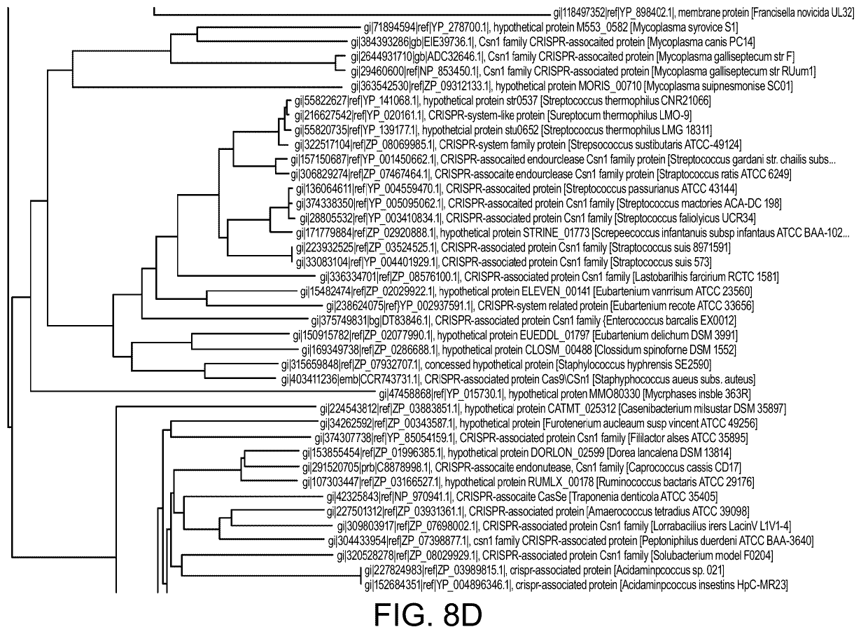

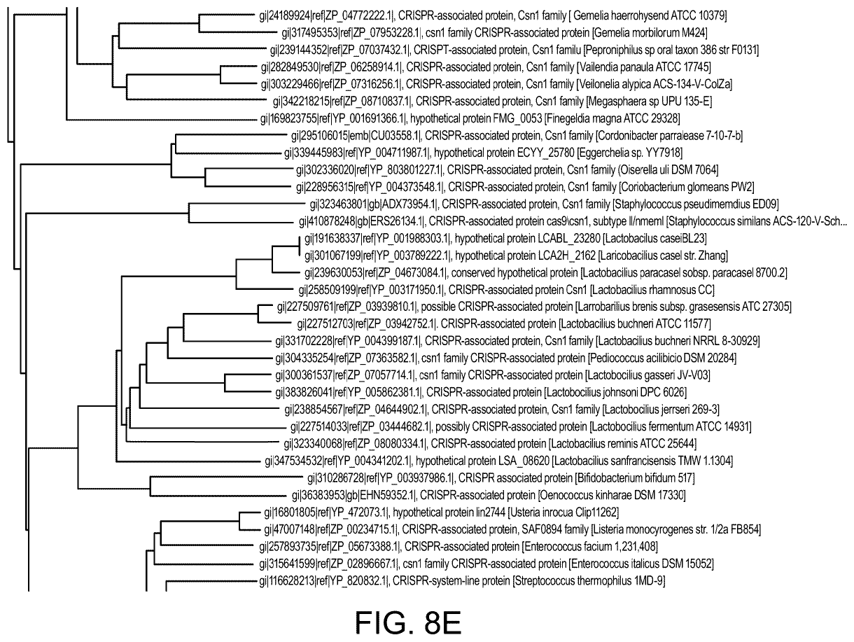

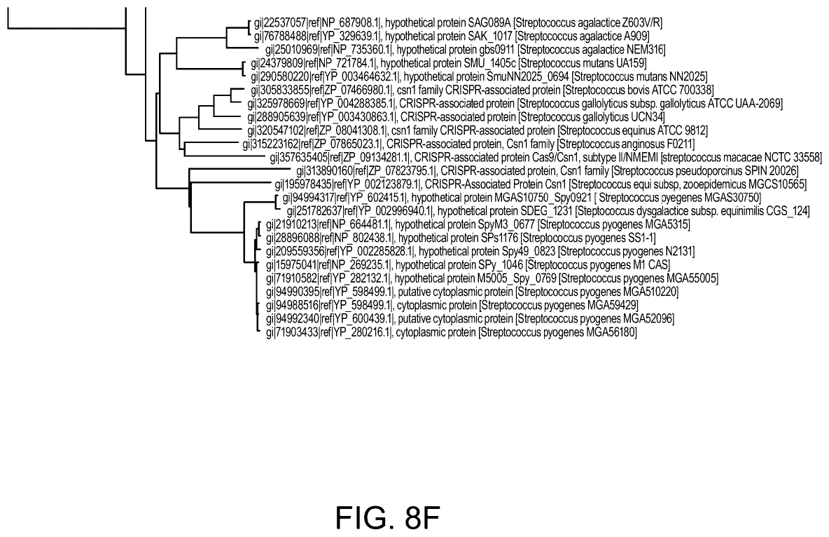

The CRISPR-Cas system is an adaptive immune mechanism against invading exogenous DNA employed by diverse species across bacteria and archaea. The type II CRISPR-Cas9 system consists of a set of genes encoding proteins responsible for the “acquisition” of foreign DNA into the CRISPR locus, as well as a set of genes encoding the “execution” of the DNA cleavage mechanism; these include the DNA nuclease (Cas9), a non-coding transactivating cr-RNA (tracrRNA), and an array of foreign DNA-derived spacers flanked by direct repeats (crRNAs). Upon maturation by Cas9, the tracrRNA and crRNA duplex guide the Cas9 nuclease to a target DNA sequence specified by the spacer guide sequences, and mediates double-stranded breaks in the DNA near a short sequence motif in the target DNA that is required for cleavage and specific to each CRISPR-Cas system. The type II CRISPR-Cas systems are found throughout the bacterial kingdom (FIGS. 7 and 8A-F) and highly diverse in in Cas9 protein sequence and size, tracrRNA and crRNA direct repeat sequence, genome organization of these elements, and the motif requirement for target cleavage. One species may have multiple distinct CRISPR-Cas systems.

Applicants evaluated 207 putative Cas9s from bacterial species (FIG. 8A-F) identified based on sequence homology to known Cas9s and structures orthologous to known subdomains. Using the method of Example 1, Applicants will carry out a comprehensive evaluation of every possible mismatch in each position of the guide RNA for these different Cas9s to generate a model to inform the design of guide RNAs having high cleavage specificity for each based on the impact of the test position and number of mismatches in the guide RNA on cleavage efficiency for each Cas9.

The CRISPR-Cas system is amenable for achieving tissue-specific and temporally controlled targeted deletion of candidate disease genes. Examples include but are not limited to genes involved in cholesterol and fatty acid metabolism, amyloid diseases, dominant negative diseases, latent viral infections, among other disorders. Accordingly, target sequences can be in candidate disease genes, e.g.:

| | | | | | SEQ | |
|---|---|---|---|---|---|---|
| | | | | | ID | |
| Disease | GENE | SPACER | PAM | Mechanism | NO: | References |
| Hypercholes- | HMG- | GCCAAATTG | CGG | Knockout | 58 | Fluvastatin: a review of its |
| terolemia | CR | GACGACCCT | | | | pharmacology and use in the |
| | | CG | | | | management of |
| | | | | | | hypercholesterolaemia. |
| | | | | | | (Plosker GL et al. Drugs |
| | | | | | | 1996, 51(3):433-459) |
| Hypercholes- | SQLE | CGAGGAGAC | TGG | Knockout | 59 | Potential role of nonstatin |
| terolemia | | CCCCGTTTC | | | | cholesterol lowering agents |
| | | GG | | | | (Trapani et al. IUBMB Life, |
| | | | | | | Volume 63, Issue 11, pages |
| | | | | | | 964-971, November 2011) |
| Hyper- | DGAT | CCCGCCGCC | AGG | Knockout | 60 | DGAT1 inhibitors as anti- |
| lipidemia | 1 | GCCGTGGCT | | | | obesity and anti-diabetic |
| | | CG | | | | agents. (Birch AM et al. |
| | | | | | | Current Opinion in Drug |
| | | | | | | Discovery & Development |
| | | | | | | [2010, 13(4):489-496) |
| Leukemia | BCR- | TGAGCTCTA | AGG | Knockout | 61 | Killing of leukemic cells |
| | ABL | CGAGATCCA | | | | with a BCR/ABL fusion gene |
| | | CA | | | | by RNA interference |
| | | | | | | (RNAi). (Fuchs et al. |
| | | | | | | Oncogene 2002, |
| | | | | | | 21(37):5716-5724) |

Examples of a pair of guide-RNA to introduce chromosomal microdeletion at a gene locus

| | | | | SEQ | | |
|---|---|---|---|---|---|---|
| | | | | ID | | |
| Disease | GENE | SPACER | PAM | NO: | Mechanism | References |
| Hyper- | PLIN2 | CTCAAAATT | TGG | 62 | Micro- | Perilipin-2 Null Mice are |
| lipidemia | guide1 | CATACCGGT | | | deletion | Protected Against Diet-Induced |
| | | TG | | | | Obesity, Adipose Inflammation |
| | | | | | | and Fatty Liver Disease |
| | | | | | | (McManaman J L et al. The |
| | | | | | | Journal of Lipid Research, |
| | | | | | | jlr.M035063. First Published on |
| | | | | | | Feb. 12, 2013) |
| Hyper- | PLIN2 | CGTTAAACA | TGG | 63 | Micro- | |
| lipidemia | guide2 | ACAACCGGA | | | deletion | |
| | | CT | | | | |
| Hyper- | SREBP | TTCACCCCG | ggg | 64 | Micro- | Inhibition of SREBP by a Small |
| lipidemia | guide1 | CGGCGCTGA | | | deletion | Molecule, Betulin, Improves |
| | | AT | | | | Hyperlipidemia and Insulin |
| | | | | | | Resistance and Reduces |
| | | | | | | Atherosclerotic Plaques (Tang J |
| | | | | | | et al. Cell Metabolism, Volume |
| | | | | | | 13, Issue 1, 44-56, 5 Jan. |
| | | | | | | 2011) |
| Hyper- | SREBP | ACCACTACC | agg | 65 | Micro- | |
| lipidemia | guide2 | AGTCCGTCC | | | deletion | |
| | | AC | | | | |

Examples of potential HIV-1 targeted spacers adapted from
Mcintyre et al, which generated shRNAs against HIV-1
optimized for maximal coverage of HIV-1 variants.
CACTGCTTAAGCCTCGCTCGAGG (SEQ ID NO: 66)
TCACCAGCAATATTCGCTCGAGG (SEQ ID NO: 67)
CACCAGCAATATTCCGCTCGAGG (SEQ ID NO: 68)
TAGCAACAGACATACGCTCGAGG (SEQ ID NO: 69)
GGGCAGTAGTAATACGCTCGAGG (SEQ ID NO: 70)
CCAATTCCCATACATTATTGTAC (SEQ ID NO: 71)

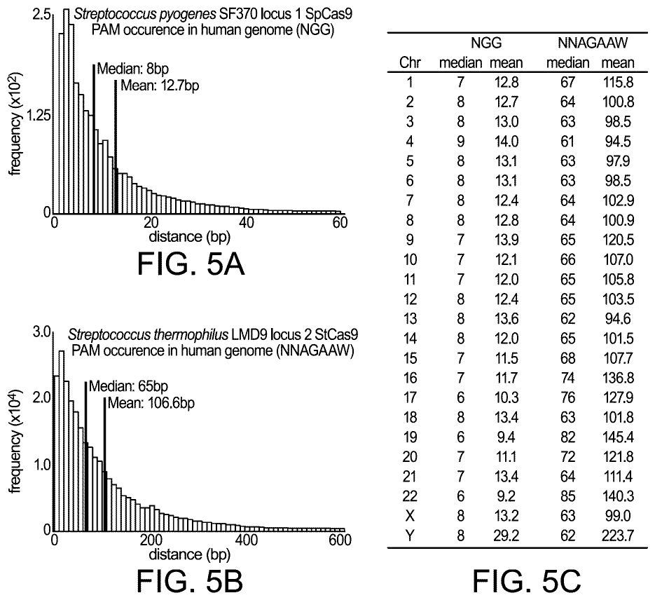

Identification of Cas9 target site: Applicants analyzed the human CFTR genomic locus and identified the Cas9 target site (PAM may contain a NGG or a NNAGAAW motif). The frequency of these PAM sequences in the human genome are shown in FIG. 5.

Protospacer IDs and their corresponding genomic target, protospacer sequence, PAM sequence, and strand location are provided in the below Table. Guide sequences were designed to be complementary to the entire protospacer sequence in the case of separate transcripts in the hybrid system, or only to the underlined portion in the case of chimeric RNAs.

TABLE
Protospacer IDs and their corresponding genomic target,
protospacer sequence, PAM sequence, and strand location

| | | | | SEQ |
|---|---|---|---|---|
| protospacer | genomic | protospacer sequence | | ID |
| ID | target | (5′ to 3′) | PAM | NO: |
| 1 | EMX1 | GGACATCGATGTCACCTCCAATGACTAG | TGG | 72 |
| | | GG | | |
| 2 | EMX1 | CATTGGAGGTGACATCGATGTCCTCCCC | TGG | 73 |
| | | AT | | |
| 3 | EMX1 | GGAAGGGCCTGAGTCCGAGCAGAAGAA | GGG | 74 |
| | | GAA | | |
| 4 | PVALB | GGTGGCGAGAGGGGCCGAGATTGGGTGT | AGG | 75 |
| | | TC | | |
| 5 | PVALB | ATGCAGGAGGGTGGCGAGAGGGGCCGA | TGG | 76 |
| | | GAT | | |

Computational identification of unique CRISPR target sites: To identify unique target sites for a Cas, e.g., a Cas9, e.g., the S. pyogenes SF370 Cas9 (SpCas9) enzyme, in nucleic acid molecules, e.g., of cells, e.g., of organisms, which include but are not limited to human, mouse, rat, zebrafish, fruit fly, and (′. elegans genome, Applicants developed a software package to scan both strands of a DNA sequence and identify all possible SpCas9 target sites. For this example, each SpCas9 target site was operationally defined as a 20 bp sequence followed by an NGG protospacer adjacent motif (PAM) sequence, and all sequences satisfying this 5′-N₂₀-NGG-3′ definition on all chromosomes were identified. To prevent non-specific genome editing, after identifying all potential sites, all target sites were filtered based on the number of times they appear in the relevant reference genome. To take advantage of sequence specificity of Cas, e.g., Cas9 activity conferred by a ‘seed’ sequence, which can be, for example, approximately 11-12 bp sequence 5′ from the PAM sequence, 5′-NNNNNNNNNN-NGG-3′ sequences were selected to be unique in the relevant genome. Genomic sequences are available on the UCSC Genome Browser and sample visualizations of the information for the Human genome hg, Mouse genome mm, Rat genome rn, Zebrafish genome danRer, D. melanogaster genome dm, C. elegans genome ce, the pig genome and cow genome are shown in FIGS. 15 through 22 respectively.

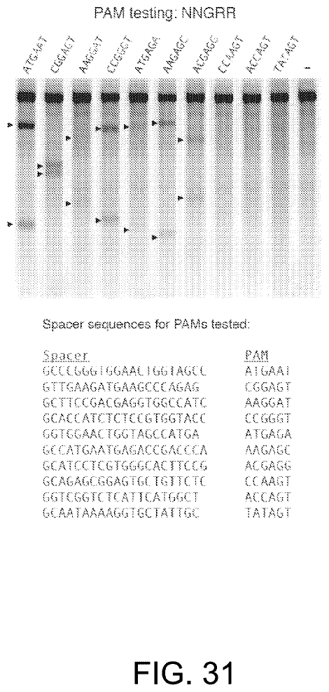

A similar analysis may be carried out for other Cas enzymes utilizing their respective PAM sequences, for e.g. Staphylococcus aureus sp. Aureus Cas9 and its PAM sequence NNGRR (FIG. 31).

### Example 4: Experimental Architecture for Evaluating CRISPR-Cas Target Activity and Specificity

Targeted nucleases such as the CRISPR-Cas systems for gene editing applications allow for highly precise modification of the genome. However, the specificity of gene editing tools is a crucial consideration for avoiding adverse off-target activity. Here, Applicants describe a Cas9 guide RNA selection algorithm that predicts off-target sites for any desired target site within mammalian genomes.

Applicants constructed large oligo libraries of guide RNAs carrying combinations of mutations to study the sequence dependence of Cas9 programming. Using next-generation deep sequencing, Applicants studied the ability of single mutations and multiple combinations of mismatches within different Cas9 guide RNAs to mediate target DNA locus modification. Applicants evaluated candidate off-target sites with sequence homology to the target site of interest to assess any off-target cleavage.

Algorithm for predicting CRISPR-Cas target activity and specificity: Data from these studies were used to develop algorithms for the prediction of CRISPR-Cas off-target activity across the human genome. The Applicants' resulting computational platform supports the prediction of all CRISPR-Cas system target activity and specificity in any genome. Applicants evaluate CRISPR-Cas activity and specificity by predicting the Cas9 cutting efficiency for any CRISPR-Cas target against all other genomic CRISPR-Cas targets, excluding constraining factors, i.e., some epigenetic modifications like repressive chromatin/heterochromatin.

The algorithms Applicants describe 1) evaluate any target site and give potential off-targets and 2) generate candidate target sites for any locus of interest with minimal predicted off-target activity.

Least squares thermodynamic model of CRISPR-Cas cutting efficiency: For arbitrary Cas9 target sites, Applicants generated a numerical thermodynamic model that predicts Cas9 cutting efficiency. Applicants propose 1) that the Cas9 guide RNA has specific free energies of hybridization to its target and any off-target DNA sequences and 2) that Cas9 modifies RNA:DNA hybridization free-energies locally in a position-dependent but sequence-independent way. Applicants trained a model for predicting CRISPR-Cas cutting efficiency based on their CRISPR-Cas guide RNA mutation data and RNA:DNA thermodynamic free energy calculations using a machine learning algorithm. Applicants then validated their resulting models by comparing their predictions of CRISPR-Cas off-target cutting at multiple genomic loci with experimental data assessing locus modification at the same sites.

The methodology adopted in developing this algorithm is as follows: The problem summary states that for arbitrary spacers and targets of constant length, a numerical model that makes thermodynamic sense and predicts Cas9 cutting efficiency is to be found.

Suppose Cas9 modifies DNA:RNA hybridization free-energies locally in a position-dependent but sequence-independent way. Then for DNA:RNA hybridization free energies ΔGᵢⱼ(k) (for position k between 1 and N) of spacer i and target j

$${Z}_{ij} = {\sum }_{k = 1}^{N} {α}_{k} Δ {G}_{ij} ( k )$$

Zᵢⱼ can be treated as an “effective” free-energy modified by the multiplicative position-weights ακ.

The “effective” free-energy Zᵢⱼ corresponds to an associated cutting-probability ˜e^{-βZ}^{ᵢⱼ} (for some constant β) in the same way that an equilibrium model of hybridization (without position-weighting) would have predicted a hybridization-probability ˜e^{-βΔG}^{ᵢⱼ}. Since cutting-efficiency has been measured, the values Zᵢⱼ can be treated as their observables. Meanwhile, ΔGᵢⱼ(k) can be calculated for any experiment's spacer-target pairing. Applicants task was to find the values αₖ, since this would allow them to estimate Zᵢⱼ for any spacer-target pair.

Writing the above equation for Zᵢⱼ in matrix form Applicants get:

$$\vec{Z} = G \vec{α} \qquad ( 1 )$$

The least-squares estimate is then

$${\vec{a}}_{est} = {( {G}^{T} G )}^{- 1} {G}^{T} \overline{Z}$$

where G^{T} is the matrix-transpose of the G and (G^{T}G)⁻¹ is the inverse of their matrix-product.

In the above G is a matrix of local DNA:RNA free-energy values whose rth row corresponds to experimental trial r and whose kth column corresponds to the kth position in the DNA:RNA hybrid tested in that experimental trial. \vec{Z} is meanwhile a column-vector whose rth row corresponds to observables from the same experimental trial as G's rth row. Because of the relation described above wherein the CRISPR cutting frequencies are estimated to vary as ˜e^{-βZ}^{ᵢⱼ}, these observables, Zᵢⱼ, were calculated as the natural logarithm of the observed cutting frequency. The observable is the cleavage efficiency of Cas, e.g., Cas9, at a target DNA for a particular guide RNA and target DNA pair. The experiment is Cas, e.g., Cas9, with a particular sgRNA/DNA target pairing, and the observable is the cleavage percentage (whether measured as indel formation percentage from cells or simply cleavage percentage in vitro) (see herein discussion on generating training data set). More in particular, every unique PCR reaction that was sequenced should be treated as a unique experimental trial to encompass replicability within the vector. This means that experimental replicates each go into separate rows of equation 1 (and because of this, some rows of G will be identical). The advantage of this is that when \vec{a} is fit, all relevant information—including replicability—is taken into account in the final estimate.

Observable \vec{Z}, values were calculated as log (observed frequency of cutting). Cutting frequencies were normalized identically (so that they all have the same “units”). For plugging in sequencing indel-frequency values, it may be best, however, to standardize sequencing depth.

The preferred way to do this would be to set a standard sequencing-depth D for which all experiments included in \vec{Z} have at least that number of reads. Since cutting frequencies below 1/D) cannot be consistently detected, this should be set as the minimum frequency for the data-set, and the values in \vec{Z} should range from log(1/D) to log(1). One could vary the value of D later on to ensure that the \vec{a} estimate isn't too dependent on the value chosen.

In a further aspect, there are different methods of graphing NGG and NNAGAAW sequences. One is with the ‘non-overlapping’ method. NGG and NRG may be regraphed in an “overlapping” fashion, as indicated in FIGS. 6A-C.

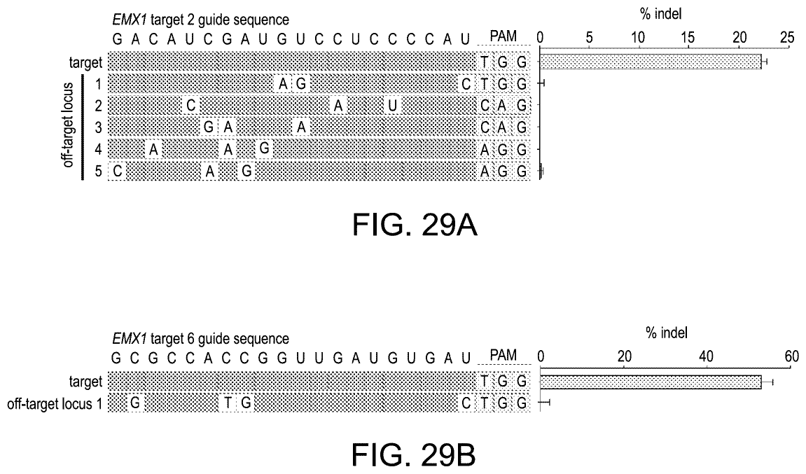

Applicants also performed a study on off target Cas9 activity as indicated in FIGS. 10, 11 and 12. Aspects of the invention also relate to predictive models that may not involve hybridization energies but instead simply use the cutting frequency information as a prediction (See FIG. 29).

### Example 5: DNA Targeting Specificity of the RNA-Guided Cas9 Nuclease

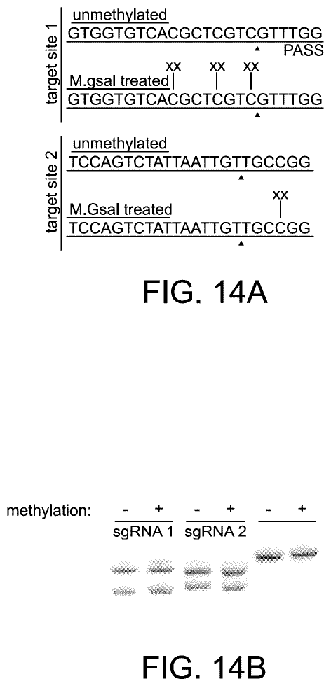

Here, Applicants report optimization of various applications of SpCas9 for mammalian genome editing and demonstrate that SpCas9-mediated cleavage is unaffected by DNA methylation (FIG. 14). Applicants further characterize SpCas9 targeting specificity using over 700 guide RNA variants and evaluate SpCas9-induced indel mutation levels at over 100 predicted genomic off-target loci. Contrary to previous models, Applicants found that SpCas9 tolerates mismatches between guide RNA and target DNA at different positions in a sequence-context dependent manner, sensitive to the number, position and distribution of mismatches. Finally, Applicants demonstrate that the dosage of SpCas9 and sgRNA can be titrated to minimize off-target modification. To facilitate mammalian genome engineering applications, Applicants used these results to establish a computational platform to guide the selection and validation of target sequences as well as off-target analyses.

The bacterial type II CRISPR system from S. pyogenes may be reconstituted in mammalian cells using three minimal components: the Cas9 nuclease (SpCas9), a specificity-determining CRISPR RNA (crRNA), and an auxiliary trans-activating crRNA (tracrRNA). Following crRNA and tracrRNA hybridization, SpCas9 is localized to the genomic target matching a 20-nt guide sequence within the crRNA, immediately upstream of a required 5′-NGG protospacer adjacent motif (PAM). Each crRNA and tracrRNA duplex may also be fused to generate a chimeric single guide RNA (sgRNA) that mimics the natural crRNA-tracrRNA hybrid. Both crRNA-tracrRNA duplexes and sgRNAs can be used to target SpCas9 for multiplexed genome editing in eukaryotic cells.

Although an sgRNA design consisting of a truncated crRNA and tracrRNA had been previously shown to mediate efficient cleavage in vitro, it failed to achieve detectable cleavage at several loci that were efficiently modified by crRNA-tracrRNA duplexes bearing identical guide sequences. Because the major difference between this sgRNA design and the native crRNA-tracrRNA duplex is the length of the tracrRNA sequence, Applicants tested whether extension of the tracrRNA tail was able to improve SpCas9 activity.

Applicants generated a set of sgRNAs targeting multiple sites within the human EMX1 and PVALB loci with different tracrRNA 3′ truncations. Using the SURVEYOR nuclease assay, Applicants assessed the ability of each Cas9 sgRNA complex to generate indels in HEK 293FT cells through the induction of DNA double-stranded breaks (DSBs) and subsequent non-homologous end joining (NHEJ) DNA damage repair (Methods and Materials). sgRNAs with +67 or +85 nucleotide (nt) tracrRNA tails mediated DNA cleavage at all target sites tested, with up to 5-fold higher levels of indels than the corresponding crRNA-tracrRNA duplexes. Furthermore, both sgRNA designs efficiently modified PVALB loci that were previously not targetable using crRNA-tracrRNA duplexes. For all five tested targets, Applicants observed a consistent increase in modification efficiency with increasing tracrRNA length. Applicants performed Northern blots for the guide RNA truncations and found increased levels expression for the longer tracrRNA sequences, suggesting that improved target cleavage was due to higher sgRNA expression or stability. Taken together, these data indicate that the tracrRNA tail is important for optimal SpCas9 expression and activity in vivo.

Applicants further investigated the sgRNA architecture by extending the duplex length from 12 to the 22 nt found in the native crRNA-tracrRNA duplex. Applicants also mutated the sequence encoding sgRNA to abolish any poly-T tracts that could serve as premature transcriptional terminators for U6-driven transcription. Applicants tested these new sgRNA scaffolds on 3 targets within the human EMX1 gene and observed only modest changes in modification efficiency. Thus, Applicants established sgRNA(+85), identical to some sgRNAs previously used, as an effective SpCas9 guide RNA architecture and used it in all subsequent studies.

Applicants have previously shown that a catalytic mutant of SpCas9 (D10A nickase) can mediate gene editing by homology-directed repair (HR) without detectable indel formation. Given its higher cleavage efficiency, Applicants tested whether sgRNA(+85), in complex with the Cas9 nickase, can likewise facilitate HR without incurring on-target NHEJ. Using single-stranded oligonucleotides (ssODNs) as repair templates, Applicants observed that both the wild-type and the D10A SpCas9 mediate HR in HEK 293FT cells, while only the former is able to do so in human embryonic stem cells. Applicants further confirmed using SURVEYOR assay that no target indel mutations are induced by the SpCas9 D10A nickase.

To explore whether the genome targeting ability of sgRNA(+85) is influenced by epigenetic factors that constrain the alternative transcription activator-like effector nuclease (TALENs) and potentially also zinc finger nuclease (ZFNs) technologies, Applicants further tested the ability of SpCas9 to cleave methylated DNA. Using either unmethylated or M. SssI-methylated pUC19 as DNA targets (FIG. 14a,b) in a cell-free cleavage assay, Applicants showed that SpCas9 efficiently cleaves pUC19 regardless of CpG methylation status in either the 20-bp target sequence or the PAM (FIG. 14c). To test whether this is also true in vivo, Applicants designed sgRNAs to target a highly methylated region of the human SERPINB5 locus. All three sgRNAs tested were able to mediate indel mutations in endogenously methylated targets.

Having established the optimal guide RNA architecture for SpCas9 and demonstrated its insensitivity to genomic CpG methylation, Applicants sought to conduct a comprehensive characterization of the DNA targeting specificity of SpCas9. Previous studies on SpCas9 cleavage specificity were limited to a small set of single-nucleotide mismatches between the guide sequence and DNA target, suggesting that perfect base-pairing within 10-12 bp directly 5′ of PAM determines Cas9 specificity, whereas PAM-distal multiple mismatches can be tolerated. In addition, a recent study using catalytically inactive SpCas9 as a transcriptional repressor found no significant off-target effects throughout the E. coli transcriptome. However, a systematic analysis of Cas9 specificity within the context of a larger mammalian genome has not yet been reported.

To address this, Applicants first evaluated the effect of imperfect guide RNA identity for targeting genomic DNA on SpCas9 activity, and then assessed the cleavage activity resulting from a single sgRNA on multiple genomic off-target loci with sequence similarity. To facilitate large scale testing of mismatched guide sequences, Applicants developed a simple sgRNA testing assay by generating expression cassettes encoding U6-driven sgRNAs by PCR and transfecting the resulting amplicons. Applicants then performed deep sequencing of the region flanking each target site for two independent biological replicates. From these data, Applicants applied a binomial model to detect true indel events resulting from SpCas9 cleavage and NHEJ misrepair and calculated 95% confidence intervals for all reported NHEJ frequencies.

Applicants used a linear model of free energy position-dependence to investigate the combined contribution of DNA:RNA sequence and mismatch-location on Cas9 cutting efficiency. While sequence composition and mismatch location alone generated Spearman correlations between estimated and observed cutting efficiencies for EMX1 target site 1 and 0.78, respectively, integration of the two parameters greatly improved this agreement, with Spearman correlation 0.86 (p<0.001). Furthermore, the incorporation of nupac RNA:RNA hybridization energies into Applicants' free energy model resulted in a 10% increase in the Spearman correlation coefficient. Taken together, the data suggests an effect of SpCas9-specific perturbations on the Watson-Crick base-pairing free energies. Meanwhile, sequence composition did not substantially improve agreement between estimated and observed cutting efficiencies for EMX1 target site 6 (Spearman correlation 0.91, p<0.001). This suggested that single mismatches in EMX1 target site 6 contributed minimally to the thermodynamic binding free energy itself.

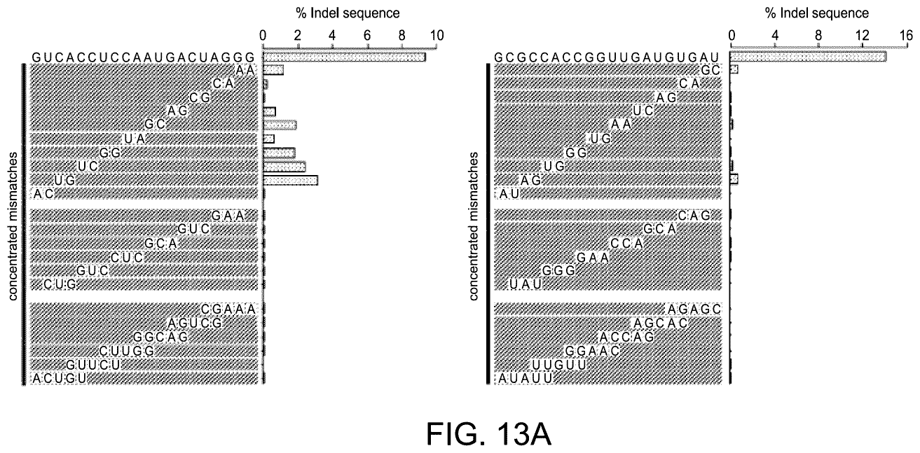

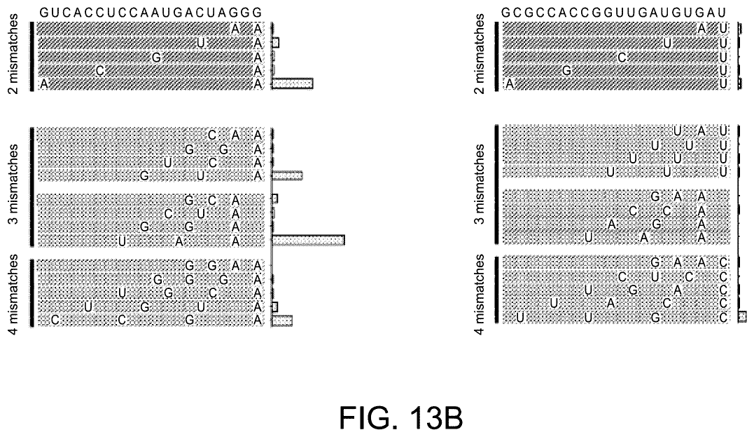

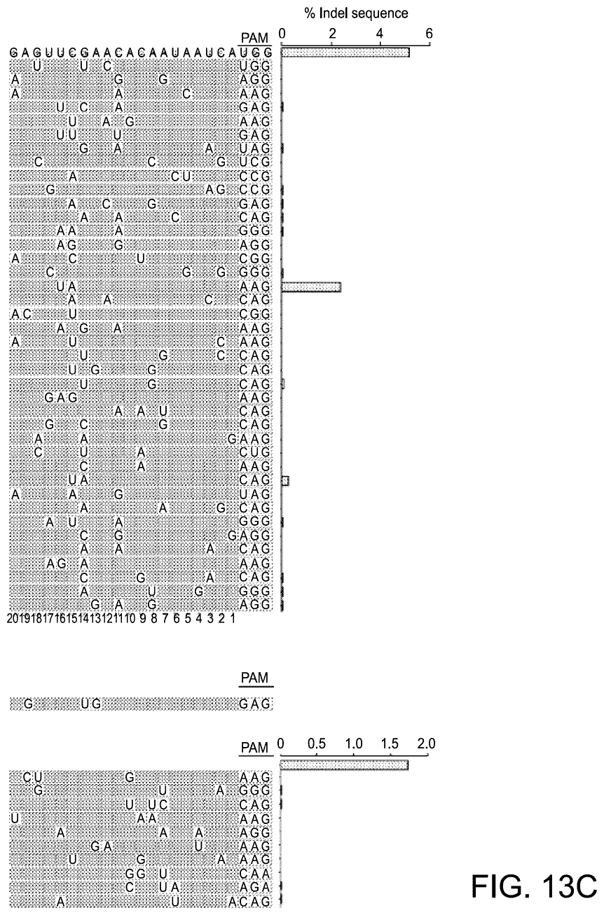

Potential genomic off-target sites with sequence similarity to a target site of interest may often have multiple base mismatches. Applicants designed a set of guide RNAs for EMX1 targets 1 and 6 that contains different combinations of mismatches to investigate the effect of mismatch number, position, and spacing on Cas9 target cleavage activity (FIG. 13a,b).

By concatenating blocks of mismatches, Applicants found that two consecutive mismatches within the PAM-proximal sequence reduced Cas9 cutting for both targets to <1% (FIG. 13a; top panels). Target site 1 cutting increased as the double mismatches shifted distally from the PAM, whereas observed cleavage for target site 6 consistently remained <0.5%. Blocks of three or five consecutive mismatches for both targets diminished Cas9 cutting to levels <0.5% regardless of position (FIG. 13, lower panels).

To investigate the effect of mismatch spacing, Applicants anchored a single PAM-proximal mutation while systematically increasing the separation between subsequent mismatches. Groups of 3 or 4 mutations each separated by 3 or fewer bases diminished Cas9 nuclease activity to levels <0.5%. However, Cas9 cutting at target site 1 increased to 3-4% when the mutations were separated by 4 or more unmutated bases (FIG. 13b). Similarly, groups of 4 mutations separated by 4 or more bases led to indel efficiencies from 0.5-1%. However, cleavage at target site 6 consistently remained below 0.5% regardless of the number or spacing of the guide RNA mismatches.

The multiple guide RNA mismatch data indicate that increasing the number of mutations diminishes and eventually abolishes cleavage. Unexpectedly, isolated mutations are tolerated as separation increased between each mismatch. Consistent with the single mismatch data, multiple mutations within the PAM-distal region are generally tolerated by Cas9 while clusters of PAM-proximal mutations are not. Finally, although the mismatch combinations represent a limited subset of base mutations, there appears to be target-specific susceptibility to guide RNA mismatches. For example, target site 6 generally showed lower cleavage with multiple mismatches, a property also reflected in its longer 12-14 bp PAM-proximal region of mutation intolerance (FIG. 12). Further investigation of Cas9 sequence-specificity may reveal design guidelines for choosing more specific DNA targets.

To determine if Applicants' findings from the guide RNA mutation data generalize to target DNA mismatches and allow the prediction of off-target cleavage within the genome, Applicants transfected cells with Cas9 and guide RNAs targeting either target 3 or target 6, and performed deep sequencing of candidate off-target sites with sequence similarity. No genomic loci with only 1 mismatch to either targets was identified. Genomic loci containing 2 or 3 mismatches relative to target 3 or target 6 revealed cleavage at some of the off-targets assessed (FIG. 13c). Targets 3 and 6 exhibited cleavage efficiencies of 7.5% and 8.0%, whereas off-target sites 3-1, 3-2, 3-4, and 3-5 were modified at 0.19%, 0.42%, 0.97%, and 0.50%, respectively. All other off-target sites cleaved at under 0.1% or were modified at levels indistinguishable from sequencing error. The off-target cutting rates were consistent with the collective results from the guide RNA mutation data: cleavage was observed at a small subset of target 3 off-targets that contained either very PAM-distal mismatches or had single mismatches separated by 4 or more bases.

Given that the genome targeting efficiencies of TALENs and ZFNs may be sensitive to confounding effects such as chromatin state or DNA methylation, Applicants sought to test whether RNA-guided SpCas9 cleavage activity would be affected by the epigenetic state of a target locus. To test this, Applicants methylated a plasmid in vitro and performed an in vitro cleavage assay on two pairs of targets containing either unmethylated or methylated CpGs. SpCas9 mediated efficient cleavage of the plasmid whether methylation occurred in the target proper or within the PAM, suggesting that SpCas9 may not be susceptible to DNA methylation effects.

The ability to program Cas9 to target specific sites in the genome by simply designing a short sgRNA has enormous potential for a variety of applications. Applicants' results demonstrate that the specificity of Cas9-mediated DNA cleavage is sequence-dependent and is governed not only by the location of mismatching bases, but also by their spacing. Importantly, while the PAM-proximal 9-12 nt of the guide sequence generally defines specificity, the PAM-distal sequences also contribute to the overall specificity of Cas9-mediated DNA cleavage. Although there are off-target cleavage sites for a given guide sequence, expected off-target sites are likely predictable based on their mismatch locations. Further work looking at the thermodynamics of sgRNA-DNA interaction will likely yield additional predictive power for off-target activity, and exploration of alternative Cas9 orthologs may also yield novel variants of Cas9s with improved specificity. Taken together, the high efficiency of Cas9 as well as its low off-target activity make CRISPR-Cas an attractive genome engineering technology.

### Example 6: Use of Cas9 to Target a Variety of Disease Types

The specificity of Cas9 orthologs can be evaluated by testing the ability of each Cas9 to tolerate mismatches between the guide RNA and its DNA target. For example, the specificity of SpCas9 has been characterized by testing the effect of mutations in the guide RNA on cleavage efficiency. Libraries of guide RNAs were made with single or multiple mismatches between the guide sequence and the target DNA. Based on these findings, target sites for SpCas9 can be selected based on the following guidelines:

To maximize SpCas9 specificity for editing a particular gene, one should choose a target site within the locus of interest such that potential ‘off-target’ genomic sequences abide by the following four constraints: First and foremost, they should not be followed by a PAM with either 5′-NGG or NAG sequences. Second, their global sequence similarity to the target sequence should be minimized. Third, a maximal number of mismatches should lie within the PAM-proximal region of the off-target site. Finally, a maximal number of mismatches should be consecutive or spaced less than four bases apart.

Similar methods can be used to evaluate the specificity of other Cas9 orthologs and to establish criteria for the selection of specific target sites within the genomes of target species.

Target selection for sgRNA: There are two main considerations in the selection of the 20-nt guide sequence for gene targeting: 1) the target sequence should precede the 5′-NGG PAM for S. pyogenes Cas9, and 2) guide sequences should be chosen to minimize off-target activity. Applicants provided an online Cas9 targeting design tool (available at the website genome-engineering.org/tools; see Examples above and FIG. 23) that takes an input sequence of interest and identifies suitable target sites. To experimentally assess off-target modifications for each sgRNA, Applicants also provide computationally predicted off-target sites for each intended target, ranked according to Applicants” quantitative specificity analysis on the effects of base-pairing mismatch identity, position, and distribution.

The detailed information on computationally predicted off-target sites is as follows: Considerations for Off-target Cleavage Activities: Similar to other nucleases, Cas9 can cleave off-target DNA targets in the genome at reduced frequencies. The extent to which a given guide sequence exhibit off-target activity depends on a combination of factors including enzyme concentration, thermodynamics of the specific guide sequence employed, and the abundance of similar sequences in the target genome. For routine application of Cas9, it is important to consider ways to minimize the degree of off-target cleavage and also to be able to detect the presence of off-target cleavage.

Minimizing off-target activity: For application in cell lines, Applicants recommend following two steps to reduce the degree of off-target genome modification. First, using Applicants' online CRISPR target selection tool, it is possible to computationally assess the likelihood of a given guide sequence to have off-target sites. These analyses are performed through an exhaustive search in the genome for off-target sequences that are similar sequences as the guide sequence. Comprehensive experimental investigation of the effect of mismatching bases between the sgRNA and its target DNA revealed that mismatch tolerance is 1) position dependent—the 8-14 bp on the 3′ end of the guide sequence are less tolerant of mismatches than the 5′ bases, 2) quantity dependent—in general more than 3 mismatches are not tolerated, 3) guide sequence dependent—some guide sequences are less tolerant of mismatches than others, and 4) concentration dependent—off-target cleavage is highly sensitive to the amount of transfected DNA. The Applicants' target site analysis web tool (available at the website genome-engineering.org/tools) integrates these criteria to provide predictions for likely off-target sites in the target genome. Second, Applicants recommend titrating the amount of Cas9 and sgRNA expression plasmid to minimize off-target activity.

Detection of off-target activities: Using Applicants' CRISPR targeting web tool, it is possible to generate a list of most likely off-target sites as well as primers performing SURVEYOR or sequencing analysis of those sites. For isogenic clones generated using Cas9, Applicants strongly recommend sequencing these candidate off-target sites to check for any undesired mutations. It is worth noting that there may be off target modifications in sites that are not included in the predicted candidate list and full genome sequence should be performed to completely verify the absence of off-target sites. Furthermore, in multiplex assays where several DSBs are induced within the same genome, there may be low rates of translocation events and can be evaluated using a variety of techniques such as deep sequencing (48).

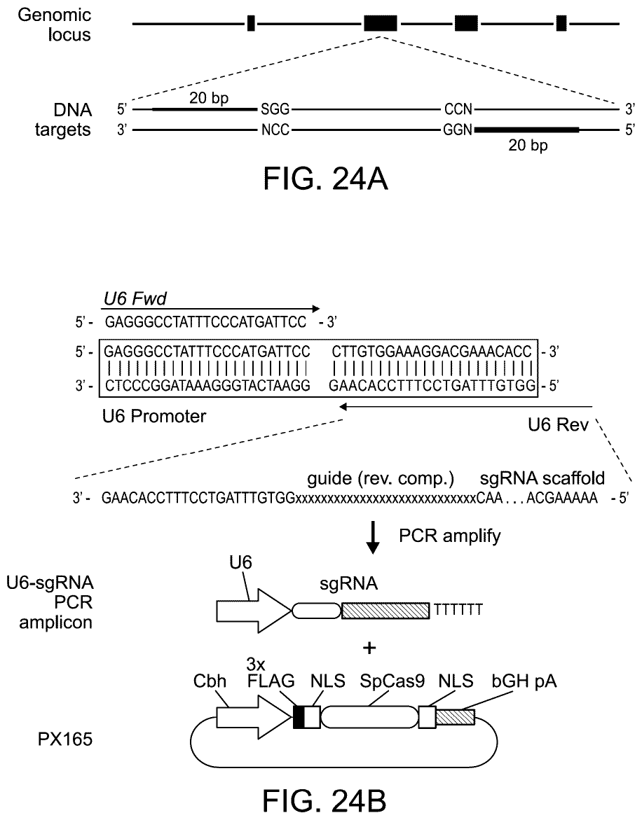

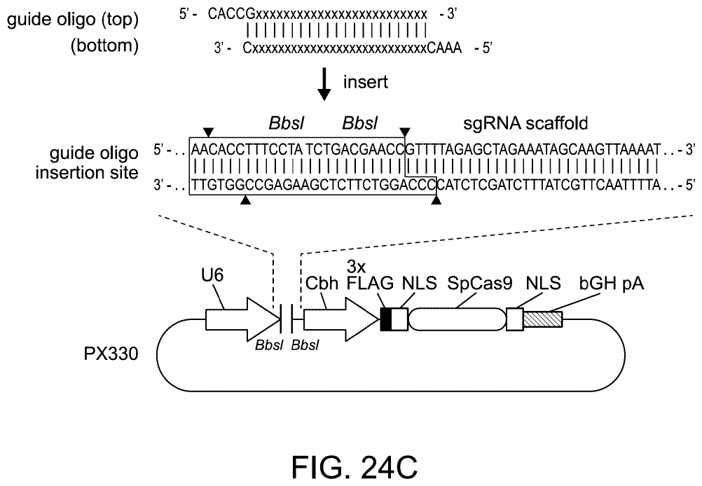

The online tool (FIG. 23) provides the sequences for all oligos and primers necessary for 1) preparing the sgRNA constructs, 2) assaying target modification efficiency, and 3) assessing cleavage at potential off-target sites. It is worth noting that because the U6 RNA polymerase III promoter used to express the sgRNA prefers a guanine (G) nucleotide as the first base of its transcript, an extra G is appended at the 5′ of the sgRNA where the 20-nt guide sequence does not begin with G (FIG. 24).

### Example 7: Base Pair Mismatching Investigations

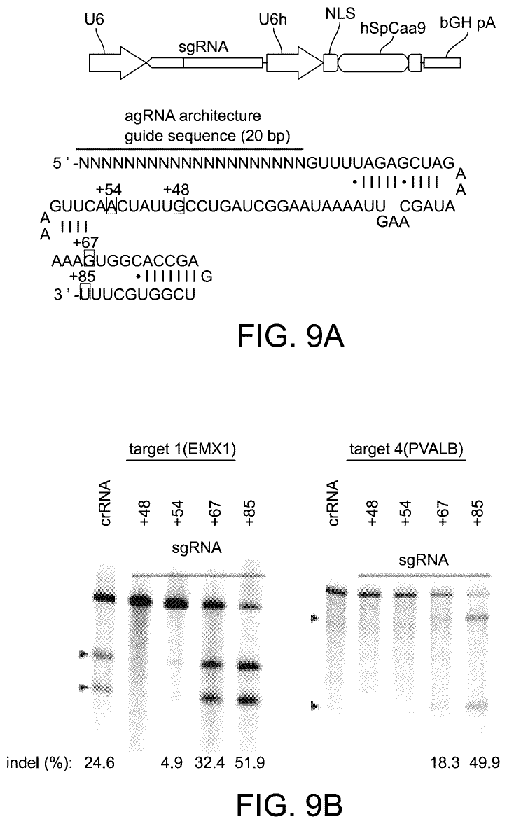

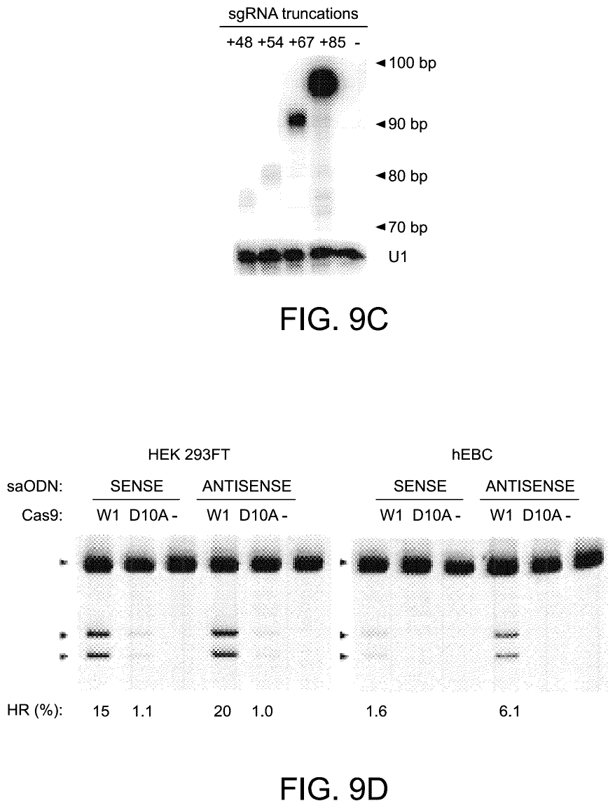

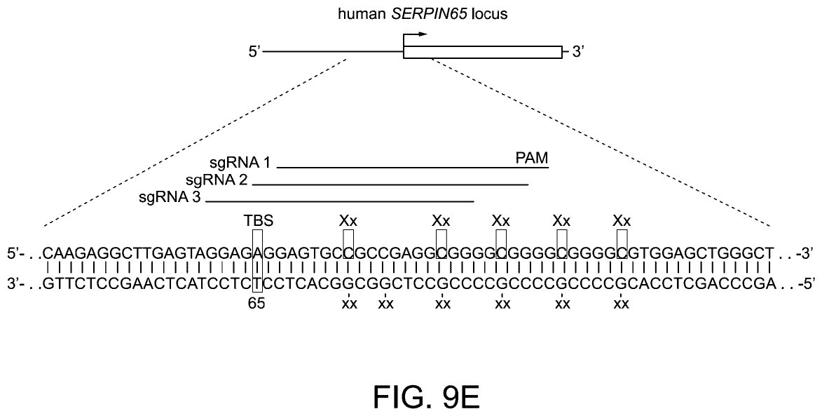

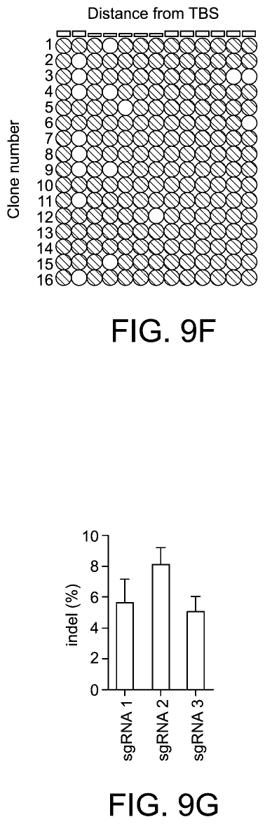

Applicants tested whether extension of the tracrRNA tail was able to improve SpCas9 activity. Applicants generated a set of sgRNAs targeting multiple sites within the human EMX1 and PVALB loci with different tracrRNA 3′ truncations (FIG. 9a). Using the SURVEYOR nuclease assay, Applicants assessed the ability of each Cas9 sgRNA complex to generate indels in HEK 293FT cells through the induction of DNA double-stranded breaks (DSBs) and subsequent non-homologous end joining (NHEJ) DNA damage repair (Methods and Materials). sgRNAs with +67 or +85 nucleotide (nt) tracrRNA tails mediated DNA cleavage at all target sites tested, with up to 5-fold higher levels of indels than the corresponding crRNA-tracrRNA duplexes (FIG. 9). Furthermore, both sgRNA designs efficiently modified PVALB loci that were previously not targetable using crRNA-tracrRNA duplexes (1) (FIG. 9b and FIG. 9b). For all five tested targets, Applicants observed a consistent increase in modification efficiency with increasing tracrRNA length. Applicants performed Northern blots for the guide RNA truncations and found increased levels expression for the longer tracrRNA sequences, suggesting that improved target cleavage was due to higher sgRNA expression or stability (FIG. 9c). Taken together, these data indicate that the tracrRNA tail is important for optimal SpCas9 expression and activity in vivo.

Applicants have previously shown that a catalytic mutant of SpCas9 (D10A nickase) can mediate gene editing by homology-directed repair (HR) without detectable indel formation. Given its higher cleavage efficiency, Applicants tested whether sgRNA(+85), in complex with the Cas9 nickase, can likewise facilitate HR without incurring on-target NHEJ. Using single-stranded oligonucleotides (ssODNs) as repair templates, Applicants observed that both the wild-type and the D10A SpCas9 mediate HR in HEK 293FT cells, while only the former is able to do so in human embryonic stem cells (hESCs; FIG. 9d).

To explore whether the genome targeting ability of sgRNA(+85) is influenced by epigenetic factors that constrain the alternative transcription activator-like effector nuclease (TALENs) and potentially also zinc finger nuclease (ZFNs) technologies, Applicants further tested the ability of SpCas9 to cleave methylated DNA. Using either unmethylated or M. SssI-methylated pUC19 as DNA targets (FIG. 14a,b) in a cell-free cleavage assay, Applicants showed that SpCas9 efficiently cleaves pUC19 regardless of CpG methylation status in either the 20-bp target sequence or the PAM. To test whether this is also true in vivo, Applicants designed sgRNAs to target a highly methylated region of the human SERPINB5 locus (FIG. 9e,f). All three sgRNAs tested were able to mediate indel mutations in endogenously methylated targets (FIG. 9g).

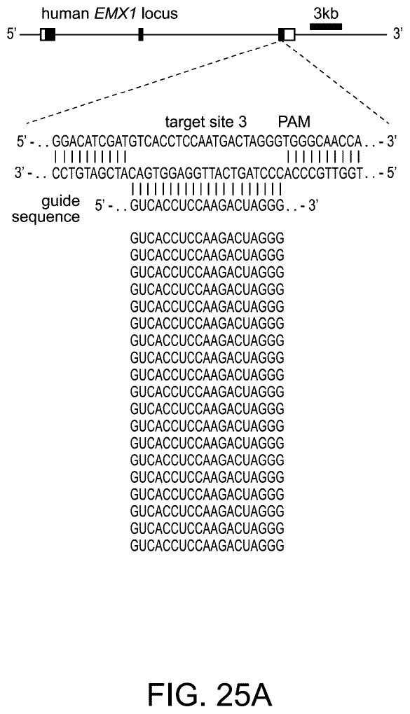

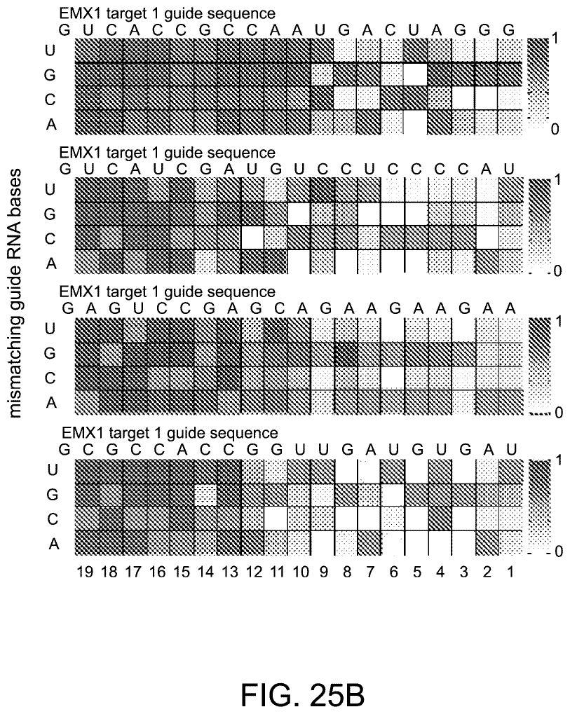

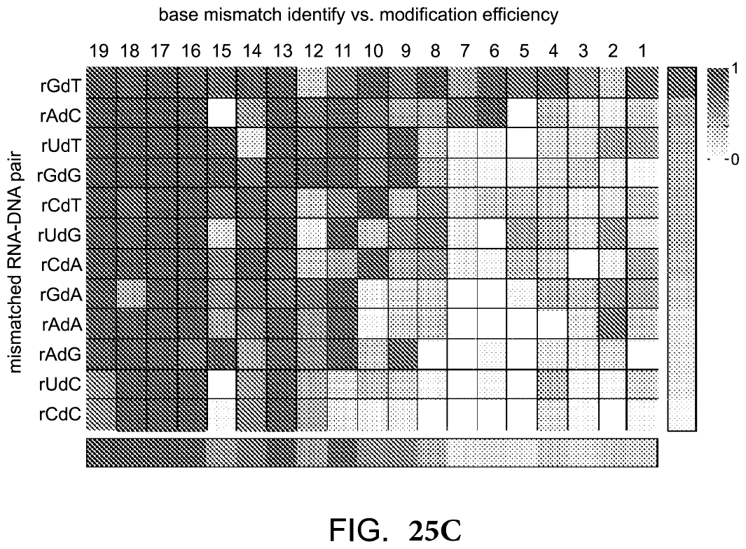

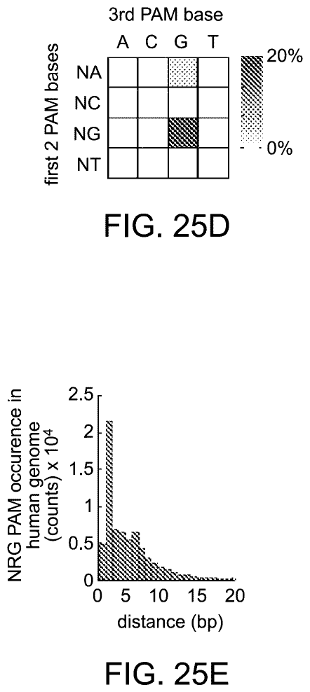

Applicants systematically investigated the effect of base-pairing mismatches between guide RNA sequences and target DNA on target modification efficiency. Applicants chose four target sites within the human EMX1 gene and, for each, generated a set of 57 different guide RNAs containing all possible single nucleotide substitutions in positions 1-19 directly 5′ of the requisite NGG PAM (FIG. 25a). The 5′ guanine at position 20 is preserved, given that the U6 promoter requires guanine as the first base of its transcript. These ‘off-target’ guide RNAs were then assessed for cleavage activity at the on-target genomic locus.

Consistent with previous findings, SpCas9 tolerates single base mismatches in the PAM-distal region to a greater extent than in the PAM-proximal region. In contrast with a model that implies a prototypical 10-12 bp PAM-proximal seed sequence that determines target specificity, Applicants found that most bases within the target site are specifically recognized, although mismatches are tolerated at different positions in a sequence-context dependent manner. Single-base specificity generally ranges from 8 to 12 bp immediately upstream of the PAM, indicating a sequence-dependent specificity boundary that varies in length (FIG. 25b).

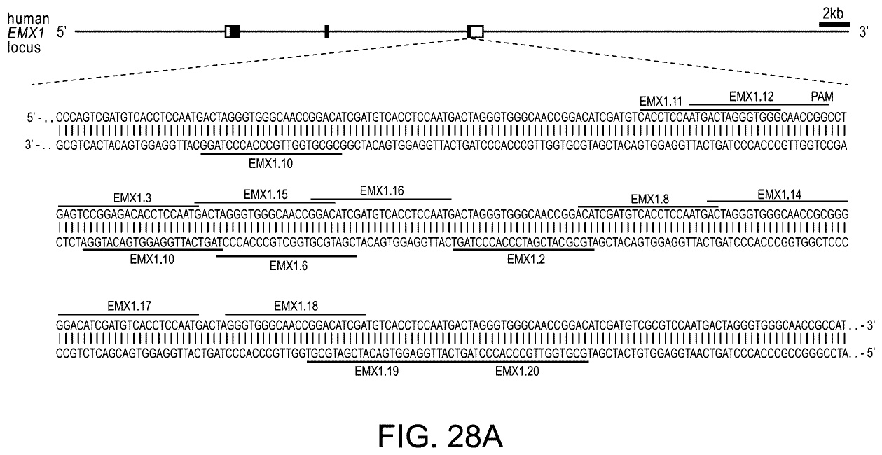

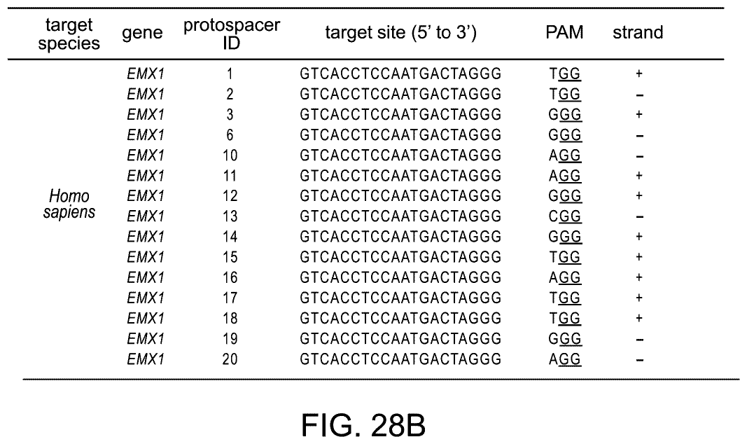

To further investigate the contributions of base identity and position within the guide RNA to SpCas9 specificity, Applicants generated additional sets of mismatched guide RNAs for eleven more target sites within the EMX1 locus (FIG. 28) totaling over 400 sgRNAs. These guide RNAs were designed to cover all 12 possible RNA:DNA mismatches for each position in the guide sequence with at least 2X coverage for positions 1-10. Applicants' aggregate single mismatch data reveals multiple exceptions to the seed sequence model of SpCas9 specificity (FIG. 25c). In general, mismatches within the 8-12 PAM-proximal bases were less tolerated by SpCas9, whereas those in the PAM-distal regions had little effect on SpCas9 cleavage. Within the PAM-proximal region, the degree of tolerance varied with the identity of a particular mismatch, with rC:dC base-pairing exhibiting the highest level of disruption to SpCas9 cleavage (FIG. 25c).

In addition to the target specificity, Applicants also investigated the NGG PAM requirement of SpCas9. To vary the second and third positions of PAM, Applicants selected 32 target sites within the EMX1 locus encompassing all 16 possible alternate PAMs with 2× coverage (Table 4). Using SURVEYOR assay, Applicants showed that SpCas9 also cleaves targets with NAG PAMs, albeit 5-fold less efficiently than target sites with NGG PAMs (FIG. 25d). The tolerance for an NAG PAM is in agreement with previous bacterial studies (12) and expands the S. pyogenes Cas9 target space to every 4-bp on average within the human genome, not accounting for constraining factors such as guide RNA secondary structure or certain epigenetic modifications (FIG. 25e).

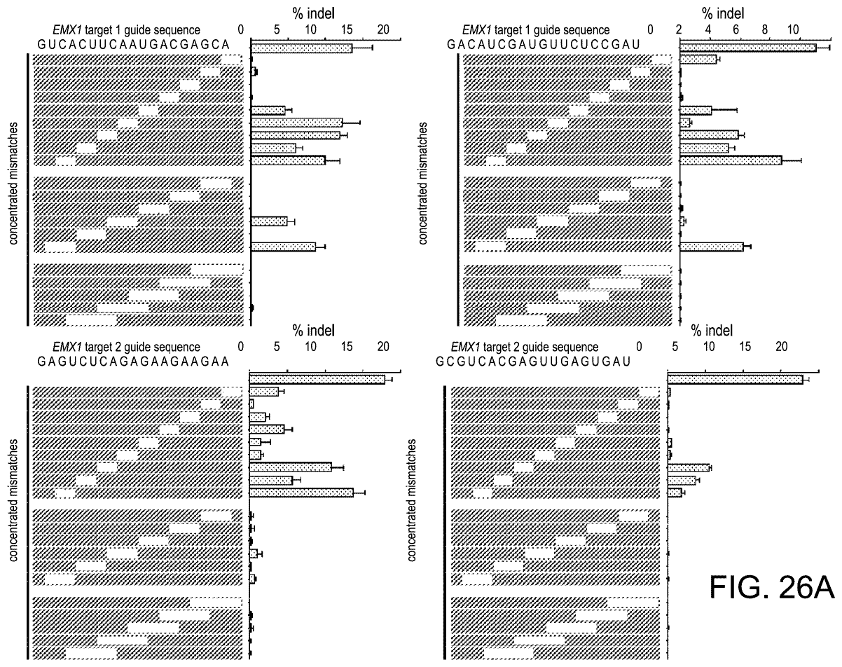

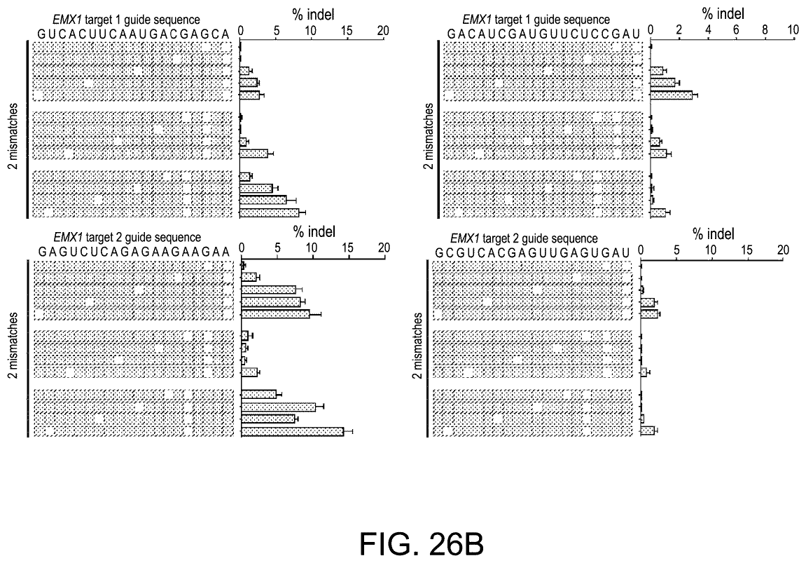

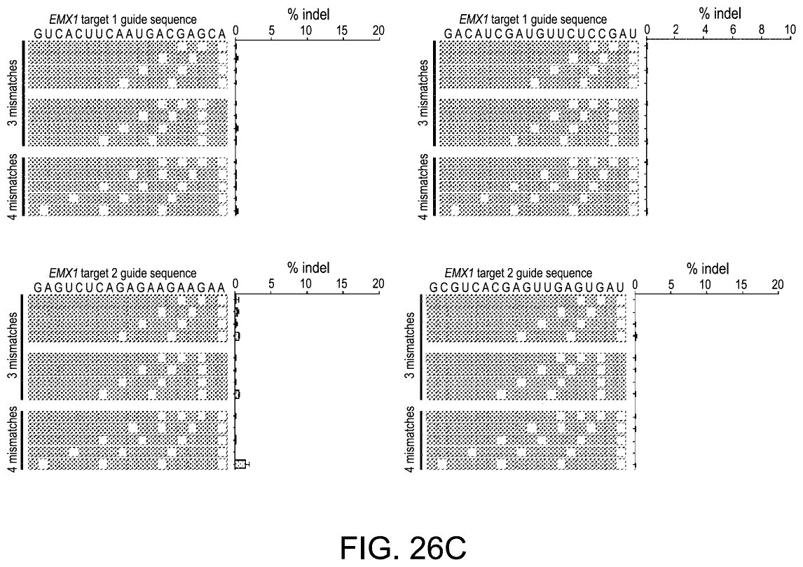

Applicants next explored the effect of multiple base mismatches on SpCas9 target activity. For four targets within the EMX1 gene, Applicants designed sets of guide RNAs that contained varying combinations of mismatches to investigate the effect of mismatch number, position, and spacing on SpCas9 target cleavage activity (FIG. 26a, b).

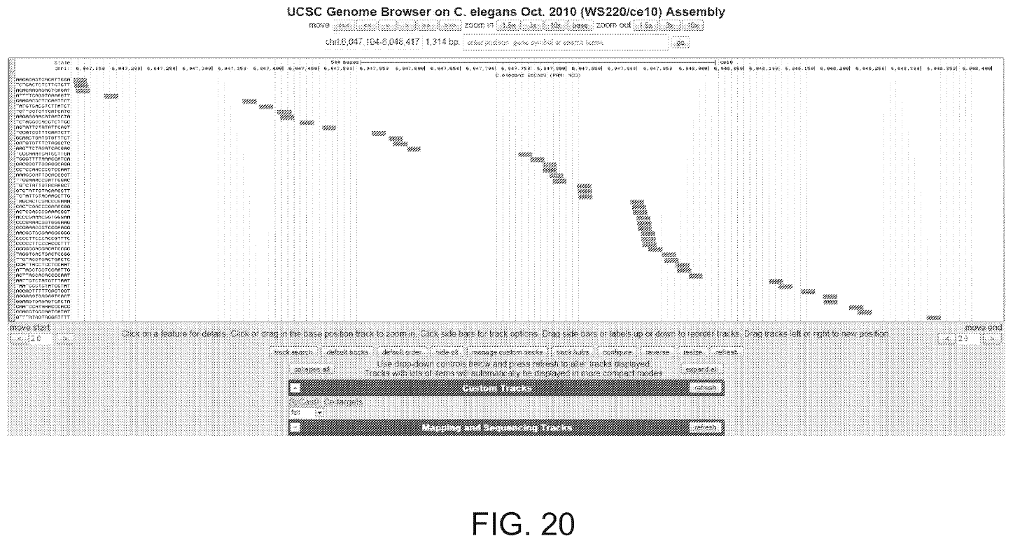

In general, Applicants observed that the total number of mismatched base-pairs is a key determinant for SpCas9 cleavage efficiency. Two mismatches, particularly those occurring in a PAM-proximal region, significantly reduced SpCas9 activity whether these mismatches are concatenated or interspaced (FIG. 26a, b); this effect is further magnified for three concatenated mismatches (FIG. 20a). Furthermore, three or more interspaced (FIG. 26c) and five concatenated (FIG. 26a) mismatches eliminated detectable SpCas9 cleavage in the vast majority of loci.

The position of mismatches within the guide sequence also affected the activity of SpCas9: PAM-proximal mismatches are less tolerated than PAM-distal counterparts (FIG. 26a), recapitulating Applicants' observations from the single base-pair mismatch data (FIG. 25c). This effect is particularly salient in guide sequences bearing a small number of total mismatches, whether those are concatenated (FIG. 26a) or interspaced (FIG. 26b). Additionally, guide sequences with mismatches spaced four or more bases apart also mediated SpCas9 cleavage in some cases (FIG. 26c). Thus, together with the identity of mismatched base-pairing, Applicants observed that many off-target cleavage effects can be explained by a combination of mismatch number and position.

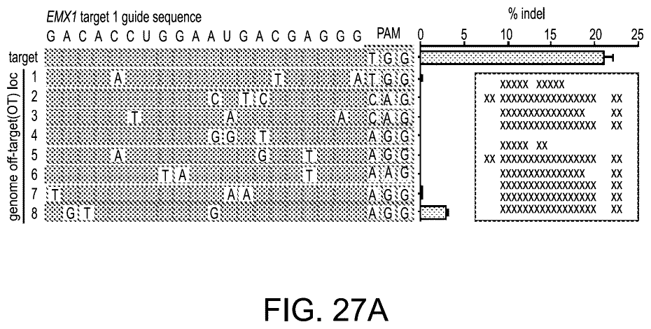

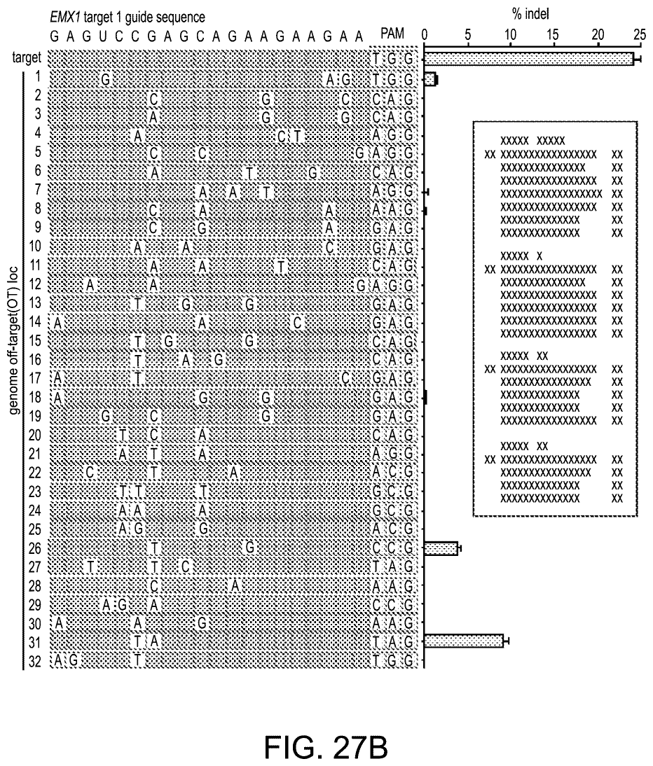

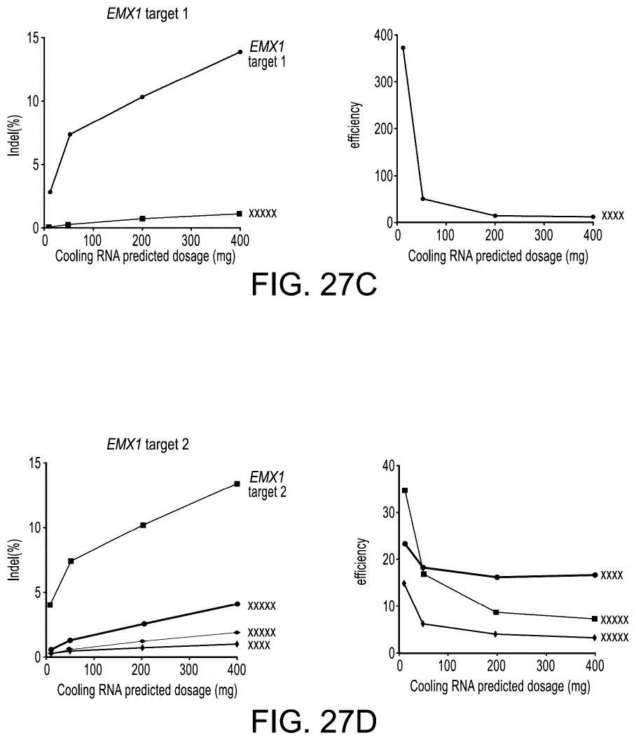

Given these mismatched guide RNA results, Applicants expected that for any particular sgRNA, SpCas9 may cleave genomic loci that contain small numbers of mismatched bases. For the four EMX1 targets described above, Applicants computationally identified 117 candidate off-target sites in the human genome that are followed by a 5′-NRG PAM and meet any of the additional following criteria: 1. up to 5 mismatches, 2. short insertions or deletions, or 3. mismatches only in the PAM-distal region. Additionally, Applicants assessed off-target loci of high sequence similarity without the PAM requirement. The majority of off-target sites tested for each sgRNA (30/31, 23/23, 48/51, and 12/12 sites for EMX1 targets 1, 2, 3, and 6, respectively) exhibited modification efficiencies at least 100-fold lower than that of corresponding on-targets (FIG. 27a, b). Of the four off-target sites identified, three contained only mismatches in the PAM-distal region, consistent with the Applicants' multiple mismatch sgRNA observations (FIG. 26). Notably, these three loci were followed by 5′-NAG PAMs, demonstrating that off-target analyses of SpCas9 must include 5′-NAG as well as 5′-NGG candidate loci.

Enzymatic specificity and activity strength are often highly dependent on reaction conditions, which at high reaction concentration might amplify off-target activity (26, 27). One potential strategy for minimizing non-specific cleavage is to limit the enzyme concentration, namely the level of SpCas9-sgRNA complex. Cleavage specificity, measured as a ratio of on- to off-target cleavage, increased dramatically as Applicants decreased the equimolar amounts of SpCas9 and sgRNA transfected into 293FT cells (FIG. 27c, d) from 7.1×10−10 to 1.8×10−11 nmol/cell (400 ng to 10 ng of Cas9-sgRNA plasmid). qRT-PCR assay confirmed that the level of hSpCas9 mRNA and sgRNA decreased proportionally to the amount of transfected DNA. Whereas specificity increased gradually by nearly 4-fold as Applicants decreased the transfected DNA amount from 7.1×10−10 to 9.0×10−11 nmol/cell (400 ng to 50 ng plasmid), Applicants observed a notable additional 7-fold increase in specificity upon decreasing transfected DNA from 9.0×10−11 to 1.8×10−11 nmol/cell (50 ng to 10 ng plasmid; FIG. 27c). These findings suggest that Applicants may minimize the level of off-target activity by titrating the amount of SpCas9 and sgRNA DNA delivered. However, increasing specificity by reducing the amount of transfected DNA also leads to a reduction in on-target cleavage. These measurements enable quantitative integration of specificity and efficiency criteria into dosage choice to optimize SpCas9 activity for different applications. Applicants further explore modifications in SpCas9 and sgRNA design that may improve the intrinsic specificity without sacrificing cleavage efficiency. FIG. 29 shows data for EMX1 target 2 and target 6. For the tested sites in FIGS. 27 and 29 (in this case, sites with 3 mismatches or less), there were no off-target sites identified (defined as off-target site cleavage within 100-fold of the on-target site cleavage).

The ability to program SpCas9 to target specific sites in the genome by simply designing a short sgRNA holds enormous potential for a variety of applications. Applicants' results demonstrate that the specificity of SpCas9-mediated DNA cleavage is sequence- and locus-dependent and governed by the quantity, position, and identity of mismatching bases. Importantly, while the PAM-proximal 8-12 bp of the guide sequence generally defines specificity, the PAM-distal sequences also contribute to the overall specificity of SpCas9-mediated DNA cleavage. Although there may be off-target cleavage for a given guide sequence, they can be predicted and likely minimized by following general design guidelines.

To maximize SpCas9 specificity for editing a particular gene, one should identify potential ‘off-target’ genomic sequences by considering the following four constraints: First and foremost, they should not be followed by a PAM with either 5′-NGG or 5′-NAG sequences. Second, their global sequence similarity to the target sequence should be minimized, and guide sequences with genomic off-target loci that have fewer than 3 mismatches should be avoided. Third, at least 2 mismatches should lie within the PAM-proximal region of the off-target site. Fourth, a maximal number of mismatches should be consecutive or spaced less than four bases apart. Finally, the amount of SpCas9 and sgRNA may be titrated to optimize on- to off-target cleavage ratio.

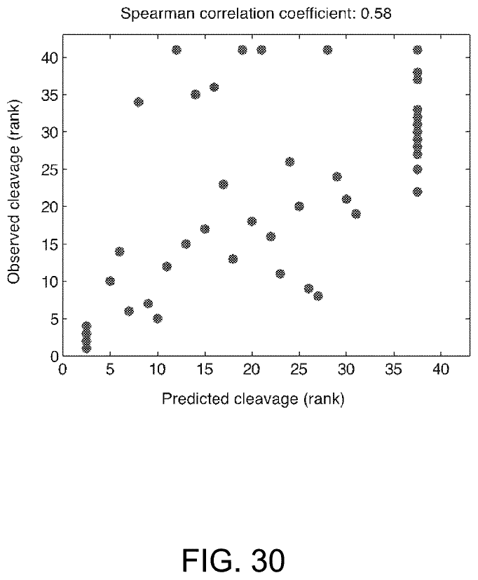

Using these criteria, Applicants formulated a simple scoring scheme to integrate the contributions of mismatch location, density, and identity for quantifying their contribution to SpCas9 cutting. Applicants applied the aggregate cleavage efficiencies of single-mismatch guide RNAs to test this scoring scheme separately on genome-wide targets. Applicants found that these factors, taken together, accounted for more than 50% of the variance in cutting-frequency rank among the genome-wide targets studied (FIG. 30).

Implementing the guidelines delineated above, Applicants designed a computational tool to facilitate the selection and validation of sgRNAs as well as to predict off-target loci for specificity analyses; this tool may be accessed at the website genome-engineering.org/tools. These results and tools further extend the SpCas9 system as a powerful and versatile alternative to ZFNs and TALENs for genome editing applications. Further work examining the thermodynamics and in vivo stability of sgRNA-DNA duplexes will likely yield additional predictive power for off-target activity, while exploration of SpCas9 mutants and orthologs may yield novel variants with improved specificity.

Accession codes All raw reads can be accessed at NCBI BioProject, accession number SRP023129.

### Methods and Materials:

Cell culture and transfection—Human embryonic kidney (HEK) cell line 293FT (Life Technologies) was maintained in Dulbecco's modified Eagle's Medium (DMEM) supplemented with 10% fetal bovine serum (HyClone), 2 mM GlutaMAX (Life Technologies), 100 U/mL penicillin, and 100 μg/mL streptomycin at 37° ° C. with 5% CO2 incubation.

293FT cells were seeded either onto 6-well plates, 24-well plates, or 96-well plates (Corning) 24 hours prior to transfection. Cells were transfected using Lipofectamine 2000 (Life Technologies) at 80-90% confluence following the manufacturer's recommended protocol. For each well of a 6-well plate, a total of 1 ug of Cas9+sgRNA plasmid was used. For each well of a 24-well plate, a total of 500 ng Cas9+sgRNA plasmid was used unless otherwise indicated. For each well of a 96-well plate, 65 ng of Cas9 plasmid was used at a 1:1 molar ratio to the U6-sgRNA PCR product.

Human embryonic stem cell line HUES9 (Harvard Stem Cell Institute core) was maintained in feeder-free conditions on GelTrex (Life Technologies) in mTesR medium (Stemcell Technologies) supplemented with 100 ug/ml Normocin (InvivoGen). HUES9 cells were transfected with Amaxa P3 Primary Cell 4-D Nucleofector Kit (Lonza) following the manufacturer's protocol.

### SURVEYOR Nuclease Assay for Genome Modification

293FT cells were transfected with plasmid DNA as described above. Cells were incubated at 37° C. for 72 hours post-transfection prior to genomic DNA extraction. Genomic DNA was extracted using the QuickExtract DNA Extraction Solution (Epicentre) following the manufacturer's protocol. Briefly, pelleted cells were resuspended in QuickExtract solution and incubated at 65° C. for 15 minutes and 98° C. for 10 minutes.

The genomic region flanking the CRISPR target site for each gene was PCR amplified (primers listed in Table 2), and products were purified using QiaQuick Spin Column (Qiagen) following the manufacturer's protocol. 400 ng total of the purified PCR products were mixed with 2 μl 10× Taq DNA Polymerase PCR buffer (Enzymatics) and ultrapure water to a final volume of 20 μl, and subjected to a re-annealing process to enable heteroduplex formation: 95° C. for 10 min, 95° C. to 85° C. ramping at −2° C./s, 85° C. to 25° C. at −0.25° C./s, and 25° ° C. hold for 1 minute. After re-annealing, products were treated with SURVEYOR nuclease and SURVEYOR enhancer S (Transgenomics) following the manufacturer's recommended protocol, and analyzed on 4-20% Novex TBE poly-acrylamide gels (Life Technologies). Gels were stained with SYBR Gold DNA stain (Life Technologies) for 30 minutes and imaged with a Gel Doc gel imaging system (Bio-rad). Quantification was based on relative band intensities.

Northern blot analysis of tracrRNA expression in human cells: Northern blots were performed as previously described. Briefly, RNAs were heated to 95° C. for 5 min before loading on 8% denaturing polyacrylamide gels (SequaGel, National Diagnostics). Afterwards, RNA was transferred to a pre-hybridized Hybond N+ membrane (GE Healthcare) and crosslinked with Stratagene UV Crosslinker (Stratagene). Probes were labeled with [gamma-32P] ATP (Perkin Elmer) with T4 polynucleotide kinase (New England Biolabs). After washing, membrane was exposed to phosphor screen for one hour and scanned with phosphorimager (Typhoon).

Bisulfite sequencing to assess DNA methylation status: HEK 293FT cells were transfected with Cas9 as described above. Genomic DNA was isolated with the DNeasy Blood & Tissue Kit (Qiagen) and bisulfite converted with EZ DNA Methylation-Lightning Kit (Zymo Research). Bisulfite PCR was conducted using KAPA2G Robust HotStart DNA Polymerase (KAPA Biosystems) with primers designed using the Bisulfite Primer Seeker (Zymo Research, Table 6). Resulting PCR amplicons were gel-purified, digested with EcoRI and HindIII, and ligated into a pUC19 backbone prior to transformation. Individual clones were then Sanger sequenced to assess DNA methylation status.

In vitro transcription and cleavage assay: HEK 293FT cells were transfected with Cas9 as described above. Whole cell lysates were then prepared with a lysis buffer (20 mM HEPES, 100 mM KCl, 5 mM MgCl2, 1 mM DTT, 5% glycerol, 0.1% Triton X-100) supplemented with Protease Inhibitor Cocktail (Roche). T7-driven sgRNA was in vitro transcribed using custom oligos (Sequences) and HiScribe T7 In Vitro Transcription Kit (NEB), following the manufacturer's recommended protocol. To prepare methylated target sites, pUC19 plasmid was methylated by M.SssI and then linearized by NheI. The in vitro cleavage assay was performed as follows: for a 20 uL cleavage reaction, 10 uL of cell lysate with incubated with 2 uL cleavage buffer (100 mM HEPES, 500 mM KCl, 25 mM MgCl2, 5 mM DTT, 25% glycerol), the in vitro transcribed RNA, and 300 ng pUC19 plasmid DNA.

Deep sequencing to assess targeting specificity: HEK 293FT cells plated in 96-well plates were transfected with Cas9 plasmid DNA and single guide RNA (sgRNA) PCR cassette 72 hours prior to genomic DNA extraction (FIG. 14). The genomic region flanking the CRISPR target site for each gene was amplified by a fusion PCR method to attach the Illumina P5 adapters as well as unique sample-specific barcodes to the target amplicons. PCR products were purified using EconoSpin 96-well Filter Plates (Epoch Life Sciences) following the manufacturer's recommended protocol.

Barcoded and purified DNA samples were quantified by Quant-iT PicoGreen dsDNA Assay Kit or Qubit 2.0 Fluorometer (Life Technologies) and pooled in an equimolar ratio. Sequencing libraries were then deep sequenced with the Illumina MiSeq Personal Sequencer (Life Technologies).

Sequencing data analysis and indel detection: MiSeq reads were filtered by requiring an average Phred quality (Q score) of at least 23, as well as perfect sequence matches to barcodes and amplicon forward primers. Reads from on- and off-target loci were analyzed by first performing Smith-Waterman alignments against amplicon sequences that included 50 nucleotides upstream and downstream of the target site (a total of 120 bp). Alignments, meanwhile, were analyzed for indels from 5 nucleotides upstream to 5 nucleotides downstream of the target site (a total of 30 bp). Analyzed target regions were discarded if part of their alignment fell outside the MiSeq read itself, or if matched base-pairs comprised less than 85% of their total length.

Negative controls for each sample provided a gauge for the inclusion or exclusion of indels as putative cutting events. For each sample, an indel was counted only if its quality score exceeded μ-σ, where μ was the mean quality-score of the negative control corresponding to that sample and σ was the standard deviation of same. This yielded whole target-region indel rates for both negative controls and their corresponding samples. Using the negative control's per-target-region-per-read error rate, q, the sample's observed indel count n, and its read-count R, a maximum-likelihood estimate for the fraction of reads having target-regions with true-indels, p, was derived by applying a binomial error model, as follows.

Letting the (unknown) number of reads in a sample having target regions incorrectly counted as having at least 1 indel be E, Applicants can write (without making any assumptions about the number of true indels)

$$Prob ( E \vert p ) = ( \begin{matrix}R ( 1 - p ) \\ E\end{matrix} ) {{q}^{E} ( 1 - q )}^{R ( 1 - p ) - E}$$

since R(1−p) is the number of reads having target-regions with no true indels. Meanwhile, because the number of reads observed to have indels is “, n=E+Rp, in other words the number of reads having target-regions with errors but no true indels plus the number of reads whose target-regions correctly have indels. Applicants can then re-write the above

$$Prob ( E \vert p ) = Prob ( n = E + Rp \vert p ) = ( \begin{matrix}R ( 1 - p ) \\ n - Rp\end{matrix} ) {{q}^{n - Rp} ( 1 - q )}^{R - n}$$

Taking all values of the frequency of target-regions with true-indels P to be equally probable a priori, Prob(n|p)∝Prob(p|n). The maximum-likelihood estimate (MLE) for the frequency of target regions with true-indels was therefore set as the value of” that maximized Prob(n|p). This was evaluated numerically.

In order to place error bounds on the true-indel read frequencies in the sequencing libraries themselves, Wilson score intervals (2) were calculated for each sample, given the MLE-estimate for true-indel target-regions, Rp, and the number of reads R. Explicitly, the lower bound l and upper bound u were calculated as

$$\begin{matrix}l = ( Rp + \frac{{z}^{2}}{2} - z \sqrt{Rp ( 1 - p ) + {z}^{2} / 4} ) / ( R + {z}^{2} ) \\ u = ( Rp + \frac{{z}^{2}}{2} + z \sqrt{Rp ( 1 - p ) + {z}^{2} / 4} ) / ( R + {z}^{2} )\end{matrix}$$

where z, the standard score for the confidence required in normal distribution of variance 1, was set to 1.96, meaning a confidence of 95%.

qRT-PCR analysis of relative Cas9 and sgRNA expression: 293FT cells plated in 24-well plates were transfected as described above. 72 hours post-transfection, total RNA was harvested with miRNeasy Micro Kit (Qiagen). Reverse-strand synthesis for sgRNAs was performed with qScript Flex cDNA kit (VWR) and custom first-strand synthesis primers (Table 6). qPCR analysis was performed with Fast SYBR Green Master Mix (Life Technologies) and custom primers (Table 2), using GAPDH as an endogenous control. Relative quantification was calculated by the ΔΔCT method.

TABLE 1
Target site sequences. Tested target sites
for S. pyogenes type II CRISPR system with
the requisite PAM. Cells were transfected
with Cas9 and either crRNA-tracrRNA or
chimeric sgRNA for each target.

| Target | | | SEQ | |
|---|---|---|---|---|
| site | genomic | Target site sequence | ID | |
| ID | target | (5′ to 3′) | NO: | PAM |
| 1 | EMX1 | GTCACCTCCAATGACTAGGG | 77 | TGG |
| 2 | EMX1 | GACATCGATGTCCTCCCCAT | 78 | TGG |
| 3 | EMX1 | GAGTCCGAGCAGAAGAAGAA | 79 | GGG |
| 6 | EMX1 | GCGCCACCGGTTGATGTGAT | 80 | GGG |
| 10 | EMX1 | GGGGCACAGATGAGAAACTC | 81 | AGG |
| 11 | EMX1 | GTACAAACGGCAGAAGCTGG | 82 | AGG |
| 12 | EMX1 | GGCAGAAGCTGGAGGAGGAA | 83 | GGG |
| 13 | EMX1 | GGAGCCCTTCTTCTTCTGCT | 84 | CGG |
| 14 | EMX1 | GGGCAACCACAAACCCACGA | 85 | GGG |
| 15 | EMX1 | GCTCCCATCACATCAACCGG | 86 | TGG |
| 16 | EMX1 | GTGGCGCATTGCCACGAAGC | 87 | AGG |
| 17 | EMX1 | GGCAGAGTGCTGCTTGCTGC | 88 | TGG |
| 18 | EMX1 | GCCCCTGCGTGGGCCCAAGC | 89 | TGG |
| 19 | EMX1 | GAGTGGCCAGAGTCCAGCTT | 90 | GGG |
| 20 | EMX1 | GGCCTCCCCAAAGCCTGGCC | 91 | AGG |
| 4 | PVALB | GGGGCCGAGATTGGGTGTTC | 92 | AGG |
| 5 | PVALB | GTGGCGAGAGGGGCCGAGAT | 93 | TGG |
| 1 | SERPINB5 | GAGTGCCGCCGAGGCGGGGC | 94 | GGG |
| 2 | SERPINB5 | GGAGTGCCGCCGAGGCGGGG | 95 | CGG |
| 3 | SERPINB5 | GGAGAGGAGTGCCGCCGAGG | 96 | CGG |

TABLE 2
Primer sequences
SURVEYOR assay

| | | | SEQ |
|---|---|---|---|
| | genomic | primer sequence | ID |
| primer name | target | (5′ to 3′) | NO: |
| Sp-EMX1-F1 | EMX1 | AAAACCACCCTTCTCTCTGGC | 97 |
| Sp-EMX1-R1 | EMX1 | GGAGATTGGAGACACGGAGAG | 98 |
| Sp-EMX1-F2 | EMX1 | CCATCCCCTTCTGTGAATGT | 99 |
| Sp-EMX1-R2 | EMX1 | GGAGATTGGAGACACGGAGA | 100 |
| Sp-PVALB-F | PVALB | CTGGAAAGCCAATGCCTGAC | 101 |
| Sp-PVALB-R | PVALB | GGCAGCAAACTCCTTGTCCT | 102 |
| | | | SEQ |
| | | | ID |

| | | |
|---|---|---|
| primer name | primer sequence (5′ to 3′) | NO: |

qRT-PCR for Cas9 and sgRNA expression

| | | |
|---|---|---|
| sgRNA reverse- | AAGCACCGACTCGGTGCCAC | 103 |
| strand synthesis | | |
| EMX1.1 sgRNA | TCACCTCCAATGACTAGGGG | 104 |
| qPCR F | | |
| EMX1.1 sgRNA | CAAGTTGATAACGGACTAGCCT | 105 |
| qPCR R | | |
| EMX1.3 sgRNA | AGTCCGAGCAGAAGAAGAAGTTT | 106 |
| qPCR F | | |
| EMX1.3 sgRNA | TTTCAAGTTGATAACGGACTAGCCT | 107 |
| qPCR R | | |
| Cas9 qPCR F | AAACAGCAGATTCGCCTGGA | 108 |
| Cas9 qPCR R | TCATCCGCTCGATGAAGCTC | 109 |
| GAPDH qPCR F | TCCAAAATCAAGTGGGGCGA | 110 |
| GAPDH qPCR R | TGATGACCCTTTTGGCTCCC | 111 |

Bisulfite PCR and sequencing

| | | |
|---|---|---|
| Bisulfite PCR F | GAGGAATTCTTTTTTTGTTYGAAT | 112 |
| (SERPINB5 locus) | ATGTTGGAGGTTTTTTGGAAG | |
| Bisulfite PCR R | GAGAAGCTTAAATAAAAAACRAC | 113 |
| (SERPINB5 locus) | AATACTCAACCCAACAACC | |
| pUC19 sequencing | CAGGAAACAGCTATGAC | 114 |

TABLE 3
Sequences for primers to test sgRNA architecture.
Primers hybridize to the reverse strand of the U6
promoter unless otherwise indicated. The U6 priming
site is in bold, the guide sequence is indicated by
the stretch of “N”s, the direct repeat sequence is
in italics, and the tracrRNA sequence is underlined.
The secondary structure of each sgRNA architecture
is shown in FIG. 71.

| | | SEQ |
|---|---|---|
| | | ID |
| primer name | primer sequence (5′ to 3′) | NO: |
| U6-Forward | GCCTCTAGAGGTACCTGAGGGCCTATTTCCCAT | 115 |
| | GATTCC | |
| I: sgRNA(DR + | ACCTCTAGAAAAAAAGCACCGACTCGGTGCCACT | 116 |
| 12, tracrRNA + | TTTTCAAGTTGATAACGGACTAGCCTTATTTTAAC | |
| 85) | TTGCTATTTCTAGCTCTAAAACNNNNNNNNNNNNN | |
| | NNNNNNNGGTGTTTCGTCCTTTCCACAAG | |
| II: sgRNA(DR + | ACCTCTAGAAAAAAAGCACCGACTCGGTGCCACT | 117 |
| 12, tracrRNA + | TTTTCAAGTTGATAACGGACTAGCCTTATATTAAC | |
| 85) mut2 | TTGCTATTTCTAGCTCTAATACNNNNNNNNNNNNNN | |
| | NNNNNNGGTGTTTCGTCCTTTCCACAAG | |
| III: sgRNA(DR + | ACCTCTAGAAAAAAAGCACCGACTCGGTGCCACT | 118 |
| 22, tracrRNA + | TTTTCAAGTTGATAACGGACTAGCCTTATTTTAAC | |
| 85) | TTGCTATGCTGTTTTGTTTCCAAAACAGCATAGCTCT | |
| | AAAACNNNNNNNNNNNNNNNNNNNNGGTGTTTC | |
| | GTCCTTTCCACAAG | |
| IV: | ACCTCTAGAAAAAAAGCACCGACTCGGTGCCACT | 119 |
| sgRNA(DR + | TTTTCAAGTTGATAACGGACTAGCCTTATATTAAC | |
| 22, tracrRNA + | TTGCTATGCTGTATTGTTTCCAATACAGCATAGCTCT | |
| 85) mut4 | AATACNNNNNNNNNNNNNNNNNNNNGGTGTTTC | |
| | GTCCTTTCCACAAG | |

TABLE 4
Target sites with alternate PAMs
for testing PAM specificity of
Cas9. All target sites for PAM
specificity testing are found
within the human EMX1 locus.

| | | SEQ |
|---|---|---|
| Target site sequence | | ID |
| (5′ to 3′) | PAM | NO: |
| AGGCCCCAGTGGCTGCTCT | NAA | 28 |
| ACATCAACCGGTGGCGCAT | NAT | 29 |
| AAGGTGTGGTTCCAGAACC | NAC | 30 |
| CCATCACATCAACCGGTGG | NAG | 31 |
| AAACGGCAGAAGCTGGAGG | NTA | 32 |
| GGCAGAAGCTGGAGGAGGA | NTT | 33 |
| GGTGTGGTTCCAGAACCGG | NTC | 34 |
| AACCGGAGGACAAAGTACA | NTG | 35 |
| TTCCAGAACCGGAGGACAA | NCA | 36 |
| GTGTGGTTCCAGAACCGGA | NCT | 37 |
| TCCAGAACCGGAGGACAAA | NCC | 38 |
| CAGAAGCTGGAGGAGGAAG | NCG | 39 |
| CATCAACCGGTGGCGCATT | NGA | 40 |
| GCAGAAGCTGGAGGAGGAA | NGT | 41 |
| CCTCCCTCCCTGGCCCAGG | NGC | 42 |
| TCATCTGTGCCCCTCCCTC | NAA | 43 |
| GGGAGGACATCGATGTCAC | NAT | 44 |
| CAAACGGCAGAAGCTGGAG | NAC | 45 |
| GGGTGGGCAACCACAAACC | NAG | 46 |
| GGTGGGCAACCACAAACCC | NTA | 47 |
| GGCTCCCATCACATCAACC | NTT | 48 |
| GAAGGGCCTGAGTCCGAGC | NTC | 49 |
| CAACCGGTGGCGCATTGCC | NTG | 50 |
| AGGAGGAAGGGCCTGAGTC | NCA | 51 |
| AGCTGGAGGAGGAAGGGCC | NCT | 52 |
| GCATTGCCACGAAGCAGGC | NCC | 53 |
| ATTGCCACGAAGCAGGCCA | NCG | 54 |
| AGAACCGGAGGACAAAGTA | NGA | 55 |
| TCAACCGGTGGCGCATTGC | NGT | 56 |
| GAAGCTGGAGGAGGAAGGG | NGC | 57 |

### Sequences

All sequences are in the 5′ to 3′ direction. For U6 transcription, the string of underlined Ts serve as the transcriptional terminator.

> U6-short tracrRNA (Streptococcus pyogenes SF370)
(SEQ ID NO: 120)
Gagggcctatttcccatgattccttcatatttgcatatacgatacaaggctgttagagagataattggaattaatttgactgtaaacacaaagata
ttagtacaaaatacgtgacgtagaaagtaataatttcttgggtagtttgcagttttaaaattatgttttaaaatggactatcatatgcttaccgtaact
tgaaagtatttcgatttcttggctttatatatcttgtggaaaggacgaaacaccGGAACCATTCAAAACAGCATAGCA
AGTTAAAATAAGGCTAGTCCGTTATCAACTTGAAAAAGTGGCACCGAGTCGGT
GCTTTTTTT
(tracrRNA sequence is in bold)
>U6-DR-guide sequence-DR (Streptococcus pyogenes SF370)
(SEQ ID NO: 121)
Gagggcctatttcccatgattccttcatatttgcatatacgatacaaggctgttagagagataattggaattaatttgactgtaaacacaaagata
ttagtacaaaatacgtgacgtagaaagtaataatttcttgggtagtttgcagttttaaaattatgttttaaaatggactatcatatgcttaccgtaact
tgaaagtatttcgatttcttggctttatatatcttgtggaaaggacgaaacaccgggttttagagctatgctgttttgaatggtcccaaaacNN
NNNNNNNNNNNNNNNNNNNNNNNNNNNNgttttagagctatgctgttttgaatggtcccaaaacTTTTTT
T
(direct repeat sequence is in italics and the guide sequence is indicated by the
stretch of “N”s)
> sgRNA containing +48 tracrRNA (Streptococcus pyogenes SF370)
(SEQ ID NO: 122)
Gagggcctatttcccatgattccttcatatttgcatatacgatacaaggctgttagagagataattggaattaatttgactgtaaacacaaagata
ttagtacaaaatacgtgacgtagaaagtaataatttcttgggtagtttgcagttttaaaattatgttttaaaatggactatcatatgcttaccgtaact
tgaaagtatttcgatttcttggctttatatatcttgtggaaaggacgaaacaccNNNNNNNNNNNNNNNNNNNNgttttaga
gctagaaatagcaagttaaaataaggctagtccgTTTTTTT
(guide sequence is highlighted in blue and the tracrRNA fragment is in bold)
> sgRNA containing +54 tracrRNA (Streptococcus pyogenes SF370)
(SEQ ID NO: 123)
Gagggcctatttcccatgattccttcatatttgcatatacgatacaaggctgttagagagataattggaattaatttgactgtaaacacaaagata
ttagtacaaaatacgtgacgtagaaagtaataatttcttgggtagtttgcagttttaaaattatgttttaaaatggactatcatatgcttaccgtaact
tgaaagtatttcgatttcttggctttatatatcttgtggaaaggacgaaacaccNNNNNNNNNNNNNNNNNNNNgttttaga
gctagaaatagcaagttaaaataaggctagtccgttatcaTTTTTTTT
(guide sequence is indicated by the stretch of “N”s and the tracrRNA fragment is in bold)
> sgRNA containing +67 tracrRNA (Streptococcus pyogenes SF370)
(SEQ ID NO: 124)
Gagggcctatttcccatgattccttcatatttgcatatacgatacaaggctgttagagagataattggaattaatttgactgtaaacacaaagata
ttagtacaaaatacgtgacgtagaaagtaataatttcttgggtagtttgcagttttaaaattatgttttaaaatggactatcatatgcttaccgtaact
tgaaagtatttcgatttcttggctttatatatcttgtggaaaggacgaaacaccNNNNNNNNNNNNNNNNNNNNgttttaga
gctagaaatagcaagttaaaataaggctagtccgttatcaacttgaaaaagtgTTTTTTT
(guide sequence is indicated by the stretch of “N”s and the tracrRNA fragment is in bold))
> sgRNA containing +85 tracrRNA (Streptococcus pyogenes SF370)
(SEQ ID NO: 125)
Gagggcctatttcccatgattccttcatatttgcatatacgatacaaggctgttagagagataattggaattaatttgactgtaaacacaaagata
ttagtacaaaatacgtgacgtagaaagtaataatttcttgggtagtttgcagttttaaaattatgttttaaaatggactatcatatgataccgtaact
tgaaagtatttcgatttcttggctttatatatcttgtggaaaggacgaaacaccNNNNNNNNNNNNNNNNNNNNgttttaga
gctagaaatagcaagttaaaataaggctagtccgttatcaacttgaaaaagtggcaccgagtcggtgaTTTTTT
(guide sequence is indicated by the stretch of “N”s and the tracrRNA fragment is in bold)
> CBh-NLS-SpCas9-NLS
(SEQ ID NO: 126)
CGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGTA
TCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTA
GTCATCGCTATTACCATGGTCGAGGTGAGCCCCACGTTCTGCTTCACTCTCCCCATCT
CCCCCCCCTCCCCACCCCCAATTTTGTATTTATTTATTTTTTAATTATTTTGTGCAGCG
ATGGGGGCGGGGGGGGGGGGGGGGCGCGCGCCAGGCGGGGCGGGGCGGGGCGAG
GGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGA
AGCGCGCGGCGGGCGGGAGTCGCTGCGACGCTGCCTTCGCCCCGTGCCCCGCTCCG
CCGCCGCCTCGCGCCGCCCGCCCCGGCTCTGACTGACCGCGTTACTCCCACAGGTGA
GCGGGCGGGACGGCCCTTCTCCTCCGGGCTGTAATTAGCTGAGCAAGAGGTAAGGG
TTTAAGGGATGGTTGGTTGGTGGGGTATTAATGTTTAATTACCTGGAGCACCTGCCT
GAAATCACTTTTTTTCAGGTTGGaccggtgccaccATGGACTATAAGGACCACGACGGAG
ACTACAAGGATCATGATATTGATTACAAAGACGATGACGATAAGATGGCCCCA
AAGAAGAAGCGGAAGGTCGGTATCCACGGAGTCCCAGCAGCCGACAAGAAGTA
CAGCATCGGCCTGGACATCGGCACCAACTCTGTGGGCTGGGCCGTGATCACCG
ACGAGTACAAGGTGCCCAGCAAGAAATTCAAGGTGCTGGGCAACACCGACCGG
CACAGCATCAAGAAGAACCTGATCGGAGCCCTGCTGTTCGACAGCGGCGAAAC
AGCCGAGGCCACCCGGCTGAAGAGAACCGCCAGAAGAAGATACACCAGACGG
AAGAACCGGATCTGCTATCTGCAAGAGATCTTCAGCAACGAGATGGCCAAGGT
GGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTAC
CACGAGAAGTACCCCACCATCTACCACCTGAGAAAGAAACTGGTGGACAGCAC
CGACAAGGCCGACCTGCGGCTGATCTATCTGGCCCTGGCCCACATGATCAAGT
TCCGGGGCCACTTCCTGATCGAGGGCGACCTGAACCCCGACAACAGCGACGTG
GACAAGCTGTTCATCCAGCTGGTGCAGACCTACAACCAGCTGTTCGAGGAAAA
CCCCATCAACGCCAGCGGCGTGGACGCCAAGGCCATCCTGTCTGCCAGACTGA
GCAAGAGCAGACGGCTGGAAAATCTGATCGCCCAGCTGCCCGGCGAGAAGAA
GAATGGCCTGTTCGGCAACCTGATTGCCCTGAGCCTGGGCCTGACCCCCAACT
TCAAGAGCAACTTCGACCTGGCCGAGGATGCCAAACTGCAGCTGAGCAAGGAC
ACCTACGACGACGACCTGGACAACCTGCTGGCCCAGATCGGCGACCAGTACGC
CGACCTGTTTCTGGCCGCCAAGAACCTGTCCGACGCCATCCTGCTGAGCGACA
TCCTGAGAGTGAACACCGAGATCACCAAGGCCCCCCTGAGCGCCTCTATGATC
AAGAGATACGACGAGCACCACCAGGACCTGACCCTGCTGAAAGCTCTCGTGCG
GCAGCAGCTGCCTGAGAAGTACAAAGAGATTTTCTTCGACCAGAGCAAGAACG
GCTACGCCGGCTACATTGACGGCGGAGCCAGCCAGGAAGAGTTCTACAAGTTC
ATCAAGCCCATCCTGGAAAAGATGGACGGCACCGAGGAACTGCTCGTGAAGCT
GAACAGAGAGGACCTGCTGCGGAAGCAGCGGACCTTCGACAACGGCAGCATCC
CCCACCAGATCCACCTGGGAGAGCTGCACGCCATTCTGCGGCGGCAGGAAGAT
TTTTACCCATTCCTGAAGGACAACCGGGAAAAGATCGAGAAGATCCTGACCTTC
CGCATCCCCTACTACGTGGGCCCTCTGGCCAGGGGAAACAGCAGATTCGCCTG
GATGACCAGAAAGAGCGAGGAAACCATCACCCCCTGGAACTTCGAGGAAGTGG
TGGACAAGGGCGCTTCCGCCCAGAGCTTCATCGAGCGGATGACCAACTTCGAT
AAGAACCTGCCCAACGAGAAGGTGCTGCCCAAGCACAGCCTGCTGTACGAGTA
CTTCACCGTGTATAACGAGCTGACCAAAGTGAAATACGTGACCGAGGGAATGA
GAAAGCCCGCCTTCCTGAGCGGCGAGCAGAAAAAGGCCATCGTGGACCTGCTG
TTCAAGACCAACCGGAAAGTGACCGTGAAGCAGCTGAAAGAGGACTACTTCAA
GAAAATCGAGTGCTTCGACTCCGTGGAAATCTCCGGCGTGGAAGATCGGTTCA
ACGCCTCCCTGGGCACATACCACGATCTGCTGAAAATTATCAAGGACAAGGAC
TTCCTGGACAATGAGGAAAACGAGGACATTCTGGAAGATATCGTGCTGACCCT
GACACTGTTTGAGGACAGAGAGATGATCGAGGAACGGCTGAAAACCTATGCCC
ACCTGTTCGACGACAAAGTGATGAAGCAGCTGAAGCGGCGGAGATACACCGGC
TGGGGCAGGCTGAGCCGGAAGCTGATCAACGGCATCCGGGACAAGCAGTCCG
GCAAGACAATCCTGGATTTCCTGAAGTCCGACGGCTTCGCCAACAGAAACTTC
ATGCAGCTGATCCACGACGACAGCCTGACCTTTAAAGAGGACATCCAGAAAGC
CCAGGTGTCCGGCCAGGGCGATAGCCTGCACGAGCACATTGCCAATCTGGCCG
GCAGCCCCGCCATTAAGAAGGGCATCCTGCAGACAGTGAAGGTGGTGGACGAG
CTCGTGAAAGTGATGGGCCGGCACAAGCCCGAGAACATCGTGATCGAAATGGC
CAGAGAGAACCAGACCACCCAGAAGGGACAGAAGAACAGCCGCGAGAGAATG
AAGCGGATCGAAGAGGGCATCAAAGAGCTGGGCAGCCAGATCCTGAAAGAACA
CCCCGTGGAAAACACCCAGCTGCAGAACGAGAAGCTGTACCTGTACTACCTGC
AGAATGGGCGGGATATGTACGTGGACCAGGAACTGGACATCAACCGGCTGTCC
GACTACGATGTGGACCATATCGTGCCTCAGAGCTTTCTGAAGGACGACTCCAT
CGACAACAAGGTGCTGACCAGAAGCGACAAGAACCGGGGCAAGAGCGACAAC
GTGCCCTCCGAAGAGGTCGTGAAGAAGATGAAGAACTACTGGCGGCAGCTGCT
GAACGCCAAGCTGATTACCCAGAGAAAGTTCGACAATCTGACCAAGGCCGAGA
GAGGCGGCCTGAGCGAACTGGATAAGGCCGGCTTCATCAAGAGACAGCTGGTG
GAAACCCGGCAGATCACAAAGCACGTGGCACAGATCCTGGACTCCCGGATGAA
CACTAAGTACGACGAGAATGACAAGCTGATCCGGGAAGTGAAAGTGATCACCC
TGAAGTCCAAGCTGGTGTCCGATTTCCGGAAGGATTTCCAGTTTTACAAAGTGC
GCGAGATCAACAACTACCACCACGCCCACGACGCCTACCTGAACGCCGTCGTG
GGAACCGCCCTGATCAAAAAGTACCCTAAGCTGGAAAGCGAGTTCGTGTACGG
CGACTACAAGGTGTACGACGTGCGGAAGATGATCGCCAAGAGCGAGCAGGAAA
TCGGCAAGGCTACCGCCAAGTACTTCTTCTACAGCAACATCATGAACTTTTTCA
AGACCGAGATTACCCTGGCCAACGGCGAGATCCGGAAGCGGCCTCTGATCGAG
ACAAACGGCGAAACCGGGGAGATCGTGTGGGATAAGGGCCGGGATTTTGCCAC
CGTGCGGAAAGTGCTGAGCATGCCCCAAGTGAATATCGTGAAAAAGACCGAGG
TGCAGACAGGCGGCTTCAGCAAAGAGTCTATCCTGCCCAAGAGGAACAGCGAT
AAGCTGATCGCCAGAAAGAAGGACTGGGACCCTAAGAAGTACGGCGGCTTCGA
CAGCCCCACCGTGGCCTATTCTGTGCTGGTGGTGGCCAAAGTGGAAAAGGGCA
AGTCCAAGAAACTGAAGAGTGTGAAAGAGCTGCTGGGGATCACCATCATGGAA
AGAAGCAGCTTCGAGAAGAATCCCATCGACTTTCTGGAAGCCAAGGGCTACAA
AGAAGTGAAAAAGGACCTGATCATCAAGCTGCCTAAGTACTCCCTGTTCGAGC
TGGAAAACGGCCGGAAGAGAATGCTGGCCTCTGCCGGCGAACTGCAGAAGGG
AAACGAACTGGCCCTGCCCTCCAAATATGTGAACTTCCTGTACCTGGCCAGCCA
CTATGAGAAGCTGAAGGGCTCCCCCGAGGATAATGAGCAGAAACAGCTGTTTG
TGGAACAGCACAAGCACTACCTGGACGAGATCATCGAGCAGATCAGCGAGTTC
TCCAAGAGAGTGATCCTGGCCGACGCTAATCTGGACAAAGTGCTGTCCGCCTA
CAACAAGCACCGGGATAAGCCCATCAGAGAGCAGGCCGAGAATATCATCCACC
TGTTTACCCTGACCAATCTGGGAGCCCCTGCCGCCTTCAAGTACTTTGACACCA
CCATCGACCGGAAGAGGTACACCAGCACCAAAGAGGTGCTGGACGCCACCCTG
ATCCACCAGAGCATCACCGGCCTGTACGAGACACGGATCGACCTGTCTCAGCT
GGGAGGCGACTTTCTTTTTCTTAGCTTGACCAGCTTTCTTAGTAGCAGCAGGAC
GCTTTAA
(NLS-hSpCas9-NLS is in bold)
> Sequencing amplicon for EMX1 guides 1.1, 1.14, 1.17
(SEQ ID NO: 127)
CCAATGGGGAGGACATCGATGTCACCTCCAATGACTAGGGTGGGCAACCACAAACC
CACGAGGGCAGAGTGCTGCTTGCTGCTGGCCAGGCCCCTGCGTGGGCCCAAGCTGG
ACTCTGGCCAC
> Sequencing amplicon for EMX1 guides 1.2, 1.16
(SEQ ID NO: 128)
CGAGCAGAAGAAGAAGGGCTCCCATCACATCAACCGGTGGCGCATTGCCACGAAGC
AGGCCAATGGGGAGGACATCGATGTCACCTCCAATGACTAGGGTGGGCAACCACAA
ACCCACGAG
> Sequencing amplicon for EMX1 guides 1.3, 1.13, 1.15
(SEQ ID NO: 129)
GGAGGACAAAGTACAAACGGCAGAAGCTGGAGGAGGAAGGGCCTGAGTCCGAGCA
GAAGAAGAAGGGCTCCCATCACATCAACCGGTGGCGCATTGCCACGAAGCAGGCCA
ATGGGGAGGACATCGAT
> Sequencing amplicon for EMX1 guides 1.6
(SEQ ID NO: 130)
AGAAGCTGGAGGAGGAAGGGCCTGAGTCCGAGCAGAAGAAGAAGGGCTCCCATCA
CATCAACCGGTGGCGCATTGCCACGAAGCAGGCCAATGGGGAGGACATCGATGTCA
CCTCCAATGACTAGGGTGG
> Sequencing amplicon for EMX1 guides 1.10
(SEQ ID NO: 131)
CCTCAGTCTTCCCATCAGGCTCTCAGCTCAGCCTGAGTGTTGAGGCCCCAGTGGCTG
CTCTGGGGGCCTCCTGAGTTTCTCATCTGTGCCCCTCCCTCCCTGGCCCAGGTGAAG
GTGTGGTTCCA
> Sequencing amplicon for EMX1 guides 1.11, 1.12
(SEQ ID NO: 132)
TCATCTGTGCCCCTCCCTCCCTGGCCCAGGTGAAGGTGTGGTTCCAGAACCGGAGGA
CAAAGTACAAACGGCAGAAGCTGGAGGAGGAAGGGCCTGAGTCCGAGCAGAAGAA
GAAGGGCTCCCATCACA
> Sequencing amplicon for EMX1 guides 1.18, 1.19
(SEQ ID NO: 133)
CTCCAATGACTAGGGTGGGCAACCACAAACCCACGAGGGCAGAGTGCTGCTTGCTG
CTGGCCAGGCCCCTGCGTGGGCCCAAGCTGGACTCTGGCCACTCCCTGGCCAGGCTT
TGGGGAGGCCTGGAGT
> Sequencing amplicon for EMX1 guides 1.20
(SEQ ID NO: 134)
CTGCTTGCTGCTGGCCAGGCCCCTGCGTGGGCCCAAGCTGGACTCTGGCCACTCCCT
GGCCAGGCTTTGGGGAGGCCTGGAGTCATGGCCCCACAGGGCTTGAAGCCCGGGGC
CGCCATTGACAGAG
>T7 promoter F primer for annealing with target strand
(SEQ ID NO: 135)
GAAATTAATACGACTCACTATAGGG
>oligo containing pUC19 target site 1 for methylation
(T7 reverse)
(SEQ ID NO: 136)
AAAAAAGCACCGACTCGGTGCCACTTTTTCAAGTTGATAACGGACTAGCCTTATTTT
AACTTGCTATTTCTAGCTCTAAAACAACGACGAGCGTGACACCACCCTATAGTGAGT
CGTATTAATTTC
>oligo containing pUC19 target site 2 for methylation
(T7 reverse)
(SEQ ID NO: 137)
AAAAAAGCACCGACTCGGTGCCACTTTTTCAAGTTGATAACGGACTAGCCTTATTTT
AACTTGCTATTTCTAGCTCTAAAACGCAACAATTAATAGACTGGACCTATAGTGAGT
CGTATTAATTTC

### REFERENCES

1. Cong, L. et al. Multiplex genome engineering using CRISPR/Cas systems. Science 339, 819-823 (2013)
2. Mali, P. et al. RNA-Guided Human Genome Engineering via Cas9. Science 339, 823-826 (2013).
3. Jinek, M. et al. RNA-programmed genome editing in human cells. eLife 2, e00471 (2013).
4. Cho, S. W., Kim, S., Kim, J. M. & Kim, J. S. Targeted genome engineering in human cells with the Cas9 RNA-guided endonuclease. Nat Biotechnol 31, 230-232 (2013).
5. Deltcheva, E. et al. CRISPR RNA maturation by trans-encoded small RNA and host factor RNase III. Nature 471, 602-607 (2011).
6. Jinek, M. et al. A programmable dual-RNA-guided DNA endonuclease in adaptive bacterial immunity. Science 337, 816-821 (2012).
7. Wang, H. et al. One-Step Generation of Mice Carrying Mutations in Multiple Genes by CRISPR/Cas-Mediated Genome Engineering. Cell 153, 910-918 (2013).
8. Guschin, D. Y. et al. A rapid and general assay for monitoring endogenous gene modification. Methods Mol Biol 649, 247-256 (2010).
9. Bogenhagen, D. F. & Brown, D. D. Nucleotide sequences in Xenopus 5S DNA required for transcription termination. Cell 24, 261-270 (1981).
10. Hwang, W. Y. et al. Efficient genome editing in zebrafish using a CRISPR-Cas system. Nat Biotechnol 31, 227-229 (2013).
11. Bultmann, S. et al. Targeted transcriptional activation of silent oct4 pluripotency gene by combining designer TALEs and inhibition of epigenetic modifiers. Nucleic Acids Res 40, 5368-5377 (2012).
12. Valton, J. et al. Overcoming transcription activator-like effector (TALE) DNA binding domain sensitivity to cytosine methylation. J Biol Chem 287, 38427-38432 (2012).
13. Christian, M. et al. Targeting DNA double-strand breaks with TAL effector nucleases. Genetics 186, 757-761 (2010).
14. Miller, J. C. et al. A TALE nuclease architecture for efficient genome editing. Nat Biotechnol 29, 143-148 (2011).
15. Mussolino, C. et al. A novel TALE nuclease scaffold enables high genome editing activity in combination with low toxicity. Nucleic acids research 39, 9283-9293 (2011).
16. Hsu, P. D. & Zhang, F. Dissecting neural function using targeted genome engineering technologies. ACS chemical neuroscience 3, 603-610 (2012).
17. Sanjana, N. E. et al. A transcription activator-like effector toolbox for genome engineering. Nature protocols 7, 171-192 (2012).
18. Porteus, M. H. & Baltimore, D. Chimeric nucleases stimulate gene targeting in human cells. Science 300, 763 (2003).
19. Miller, J. C. et al. An improved zinc-finger nuclease architecture for highly specific genome editing. Nat Biotechnol 25, 778-785 (2007).
20. Sander, J. D. et al. Selection-free zinc-finger-nuclease engineering by context-dependent assembly (CoDA). Nat Methods 8, 67-69 (2011).
21. Wood, A. J. et al. Targeted genome editing across species using ZFNs and TALENs. Science 333, 307 (2011).
22. Bobis-Wozowicz, S., Osiak, A., Rahman, S. H. & Cathomen, T. Targeted genome editing in pluripotent stem cells using zinc-finger nucleases. Methods 53, 339-346 (2011).
23. Jiang, W., Bikard, D., Cox, D., Zhang, F. & Marraffini, L. A. RNA-guided editing of bacterial genomes using CRISPR-Cas systems. Nat Biotechnol 31, 233-239 (2013).
24. Qi, L. S. et al. Repurposing CRISPR as an RNA-Guided Platform for Sequence-Specific Control of Gene Expression. Cell 152, 1173-1183 (2013).
25. Michaelis, L. M., Maud “Die kinetik der invertinwirkung.”. Biochem. z (1913).
26. Mahfouz, M. M. et al. De novo-engineered transcription activator-like effector (TALE) hybrid nuclease with novel DNA binding specificity creates double-strand breaks. Proc Natl Acad Sci USA 108, 2623-2628 (2011).
27. Wilson, E. B. Probable inference, the law of succession, and statistical inference. J Am Stat Assoc 22, 209-212 (1927).
28. Ding, Q. et al. A TALEN genome-editing system for generating human stem cell-based disease models. Cell Stem Cell 12, 238-251 (2013).
29. Soldner, F. et al. Generation of isogenic pluripotent stem cells differing exclusively at two early onset Parkinson point mutations. Cell 146, 318-331 (2011).
30. Carlson, D. F. et al. Efficient TALEN-mediated gene knockout in livestock. Proc Natl Acad Sci USA 109, 17382-17387 (2012).
31. Geurts, A. M. et al. Knockout Rats via Embryo Microinjection of Zinc-Finger Nucleases. Science 325, 433-433 (2009).
32. Takasu, Y. et al. Targeted mutagenesis in the silkworm Bombyx mori using zinc finger nuclease mRNA injection. Insect Biochem Molec 40, 759-765 (2010).
33. Watanabe, T. et al. Non-transgenic genome modifications in a hemimetabolous insect using zinc-finger and TAL effector nucleases. Nat Commun 3 (2012).
34. Reyon, D. et al. FLASH assembly of TALENs for high-throughput genome editing. Nat Biotechnol 30, 460-465 (2012).
35. Boch, J. et al. Breaking the code of DNA binding specificity of TAL-type III effectors. Science 326, 1509-1512 (2009).
36. Moscou, M. J. & Bogdanove, A. J. A simple cipher governs DNA recognition by TAL effectors. Science 326, 1501 (2009).
37. Deveau, H., Garneau, J. E. & Moineau, S. CRISPR/Cas system and its role in phage-bacteria interactions. Annu Rev Microbiol 64, 475-493 (2010).
38. Horvath, P. & Barrangou, R. CRISPR/Cas, the immune system of bacteria and archaea. Science 327, 167-170 (2010).
39. Makarova, K. S. et al. Evolution and classification of the CRISPR-Cas systems. Nat Rev Microbiol 9, 467-477 (2011).
40. Bhaya, D., Davison, M. & Barrangou, R. CRISPR-Cas systems in bacteria and archaea: versatile small RNAs for adaptive defense and regulation. Annu Rev Genet 45, 273-297 (2011).
41. Garneau, J. E. et al. The CRISPR/Cas bacterial immune system cleaves bacteriophage and plasmid DNA. Nature 468, 67-71 (2010).
42. Gasiunas, G., Barrangou, R., Horvath, P. & Siksnys, V. Cas9-crRNA ribonucleoprotein complex mediates specific DNA cleavage for adaptive immunity in bacteria. Proc Natl Acad Sci USA 109, E2579-2586 (2012).
43. Urnov, F. D., Rebar, E. J., Holmes, M. C., Zhang, H. S. & Gregory, P. D. Genome editing with engineered zinc finger nucleases. Nat Rev Genet 11, 636-646 (2010).
44. Perez, E. E. et al. Establishment of HIV-1 resistance in CD4(+) T cells by genome editing using zinc-finger nucleases. Nat Biotechnol 26, 808-816 (2008).
45. Chen, F. Q. et al. High-frequency genome editing using ssDNA oligonucleotides with zinc-finger nucleases. Nat Methods 8, 753-U796 (2011).
46. Bedell, V. M. et al. In vivo genome editing using a high-efficiency TALEN system. Nature 491, 114-U133 (2012).
47. Saleh-Gohari, N. & Helleday, T. Conservative homologous recombination preferentially repairs DNA double-strand breaks in the S phase of the cell cycle in human cells. Nucleic Acids Res 32, 3683-3688 (2004).
48. Sapranauskas, R. et al. The Streptococcus thermophilus CRISPR/Cas system provides immunity in Escherichia coli. Nucleic Acids Res 39, 9275-9282 (2011).
49. Shen, B. et al. Generation of gene-modified mice via Cas9/RNA-mediated gene targeting. Cell Res 23, 720-723 (2013).
50. Tuschl, T. Expanding small RNA interference. Nat Biotechnol 20, 446-448 (2002).
51. Smithies, O., Gregg, R. G., Boggs, S. S., Koralewski, M. A. & Kucherlapati, R. S. Insertion of DNA sequences into the human chromosomal beta-globin locus by homologous recombination. Nature 317, 230-234 (1985).
52. Thomas, K. R., Folger, K. R. & Capecchi, M. R. High frequency targeting of genes to specific sites in the mammalian genome. Cell 44, 419-428 (1986).
53. Hasty, P., Rivera-Perez, J. & Bradley, A. The length of homology required for gene targeting in embryonic stem cells. Mol Cell Biol 11, 5586-5591 (1991).
54. Wu, S., Ying, G. X., Wu, Q. & Capecchi, M. R. A protocol for constructing gene targeting vectors: generating knockout mice for the cadherin family and beyond. Nat Protoc 3, 1056-1076 (2008).
55. Oliveira, T. Y. et al. Translocation capture sequencing: a method for high throughput mapping of chromosomal rearrangements. J Immunol Methods 375, 176-181 (2012).
56. Tremblay et al., Transcription Activator-Like Effector Proteins Induce the Expression of the Frataxin Gene, Human Gene Therapy. August 2012, 23(8): 883-890.
57. Shalek et al. Nanowire-mediated delivery enables functional interrogation of primary immune cells: application to the analysis of chronic lymphocytic leukemia. Nano Letters, 2012, Dec. 12; 12(12):6498-504.
58. Pardridge et al. Preparation of Trojan horse liposomes (THLs) for gene transfer across the blood-brain barrier; Cold Spring Harb Protoc; 2010; April; 2010 (4)
59. Plosker G L et al. Fluvastatin: a review of its pharmacology and use in the management of hypercholesterolaemia; Drugs 1996, 51(3):433-459).
60. Trapani et al. Potential role of nonstatin cholesterol lowering agents; IUBMB Life, Volume 63, Issue 11, pages 964-971, November 2011
61. Birch A M et al. DGAT1 inhibitors as anti-obesity and anti-diabetic agents; Current Opinion in Drug Discovery & Development, 2010, 13(4):489-496
62. Fuchs et al. Killing of leukemic cells with a BCR/ABL fusion gene by RNA interference (RNAi), Oncogene 2002, 21(37):5716-5724.
63. McManaman J L et al. Perilipin-2 Null Mice are Protected Against Diet-Induced Obesity, Adipose Inflammation and Fatty Liver Disease; The Journal of Lipid Research, jlr.M035063. First Published on Feb. 12, 2013.
64. Tang J et al. Inhibition of SREBP by a Small Molecule, Betulin, Improves Hyperlipidemia and Insulin Resistance and Reduces Atherosclerotic Plaques; Cell Metabolism, Volume 13, Issue 1, 44-56, 5 Jan. 2011.
65. Dumitrache et al. Trex2 enables spontaneous sister chromatid exchanges without facilitating DNA double-strand break repair; Genetics. 2011 August; 188(4): 787-797

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Parties to a Joint Research Agreement

The claimed invention was made by, on behalf of, and/or in connection with one or more of the following parties to a joint research agreement: the Broad Institute, Inc., Massachusetts Institute of Technology, and President and Fellows of Harvard College. The joint research agreement was in effect on and before the date the claimed invention was made, and the claimed invention was made as a result of activities undertaken within the scope of the joint research agreement.

1. A method of identifying one or more unique target sequences in a genome of a eukaryotic organism that are susceptible to being recognized by a CRISPR-Cas9 system, wherein the method comprises:
locating a protospacer adjacent motif (PAM) in the genome of the eukaryotic organism, wherein the PAM is recognized by a Cas9 protein;
analyzing a candidate target sequence upstream of the PAM to determine if the sequence occurs elsewhere in the genome of the eukaryotic organism;
selecting the candidate target sequence if a seed region of 12 nucleotides in the candidate target sequence immediately 5′ of the PAM does not occur elsewhere in the genome immediately 5′ of the PAM, wherein the PAM is present at multiple sites throughout the genome, thereby identifying a unique target sequence; and
expressing in a eukaryotic cell a CRISPR-Cas9 system that recognizes the unique target sequence.

2. The method of claim 1, wherein the PAM is recognized by a S. pyogenes Cas9 enzyme.

3. The method of claim 2, wherein the PAM is NGG.

4. The method of claim 1, wherein the candidate target sequence upstream of the PAM is at least 20 bp in length.

5. The method of claim 1, wherein the candidate target sequence is located in a genomic locus of interest associated with a disease or disorder.

6. The method of claim 1, wherein the PAM is recognized by a S. thermophilus CRISPR1 Cas9 enzyme.

7. The method of claim 6, wherein the PAM is NNAGAAW.

8. The method of claim 1, wherein the PAM is recognized by S. thermophilus CRISPR3 Cas9 enzyme.

9. The method of claim 8, wherein the PAM is NGGNG.

10. The method of claim 1, wherein the Cas9 protein is S. pyogenes Cas9, wherein the method comprises searching for 5′-Nₓ-NGG-3′ in the genome of the eukaryotic organism, optionally wherein x is 20.

11. The method of claim 1, wherein the Cas9 protein is S. thermophilus CRISPR1 Cas9, wherein the method comprises searching for 5′-Nₓ-NNAGAAW-3′ in the genome of the eukaryotic organism, optionally wherein x is 20.

12. The method of claim 1, wherein the Cas9 protein is S. thermophilus CRISPR3 Cas9, wherein the method comprises searching for 5′-Nₓ-NGGNG-3′ in the genome of the eukaryotic organism, optionally wherein x is 20.

13. The method of claim 1, wherein the method comprises identifying a unique target sequence MMMMMMMMNNNNNNNNNNNNXGG where NNNNNNNNNNNNXGG has a single occurrence in the genome of the eukaryotic organism;
wherein: X is A, G, T, or C; Nis A, G, T, or C; M is A, G, T, or C.

14. The method of claim 1, wherein the method comprises identifying a unique target sequence MMMMMMMMNNNNNNNNNNNNXXAGAAW where NNNNNNNNNNNNXXAGAAW has a single occurrence in the genome of the eukaryotic organism;
wherein: W is A or T; X is A, G, T, or C; Nis A, G, T, or C; M is A, G, T, or C.

15. The method of claim 1, wherein the method comprises identifying a unique target sequence MMMMMMMMNNNNNNNNNNNNXGGXG where NNNNNNNNNNNNXGGXG has a single occurrence in the genome of the eukaryotic organism;
wherein X is A, G, T, or C; Nis A, G, T, or C; M is A, G, T, or C.
